SEQUENCE LISTING (16 sequences)

SEQ ID NO: 1 - protein, 28 aa, Artificial Sequence
  SA-5
RVIRVVQRACRAIRHIVRRIRQGLRRIL

SEQ ID NO: 2 - protein, 31 aa, Artificial Sequence
  LSA-5
RVIRVVQRACRAIRHIVRRIRQGLRRILRVV

SEQ ID NO: 3 - protein, 31 aa, Artificial Sequence
  WLSA-5
RWIRVVQRWCRAIRHIWRRIRQGLRRWLRVV

SEQ ID NO: 4 - protein, 12 aa, Artificial Sequence
  LBU-1
RVVRVVRRVVRR

SEQ ID NO: 5 - protein, 24 aa, Artificial Sequence
  LBU-2
RRVVRRVRRVVRRVVRVVRRVVRR

SEQ ID NO: 6 - protein, 36 aa, Artificial Sequence
  LBU-3
VRRVVRRVVRVVRRVVRRVRRVVRRVVRVVRRVVRR

SEQ ID NO: 7 - protein, 42 aa, Artificial Sequence
  LBU-3.5
RRVVRRVRRVVRRVVRVVRRVVRRVRRVVRRVVRVVRRVVRR

SEQ ID NO: 8 - protein, 48 aa, Artificial Sequence
  LBU-4
RVVRVVRRVVRRVRRVVRRVVRVVRRVVRRVRRVVRRVVRVVRRVVRR

SEQ ID NO: 9 - protein, 12 aa, Artificial Sequence
  WLBU-1
RVVRVVRRWVRR

SEQ ID NO: 10 - protein, 24 aa, Artificial Sequence
  WLBU-2
RRWVRRVRRVWRRVVRVVRRWVRR

SEQ ID NO: 11 - protein, 36 aa, Artificial Sequence
  WLBU-3
VRRVWRRVVRVVRRWVRRVRRVWRRVVRVVRRWVRR

SEQ ID NO: 12 - protein, 48 aa, Artificial Sequence
  WLBU-4
RVVRVVRRWVRRVRRVWRRVVRVVRRWVRRVRRVWRRVVRVVRRWRVV

SEQ ID NO: 13 - protein, 6 aa, Artificial Sequence
  WR6
RRWWRR

SEQ ID NO: 14 - protein, 12 aa, Artificial Sequence
  WR12
RWWRWWRRWWRR

SEQ ID NO: 15 - protein, 18 aa, Artificial Sequence
  WR18
WRRWWRRWWRWWRRWWRR

SEQ ID NO: 16 - protein, 24 aa, Artificial Sequence
  WR24
RRWWRRWRRWWRRWWRWWRRWWRR

(19) United States
(12) Patent Application Publication
(10) Pub. No.: US 2024/0317809 A1
(43) Pub. Date: Sep. 26, 2024

(54) Engineered Antimicrobial Peptides and Usage Thereof

(71) Applicants: University of Pittsburgh-Of the Commonwealth System of Higher Education, Pittsburgh, PA (US); Peptilogics, Inc., Pittsburgh, PA (US)

(72) Inventors: Kenneth Urish, Sewickley, PA (US); Jonathan Steckbeck, Pittsburgh, PA (US); Despina X. Dobbins, Pittsburgh, PA (US); Nicholas Pachuda, Pittsburgh, PA (US); David Huang, Pittsburgh, PA (US); Bradd N. Picone, Pittsburgh, PA (US)

(21) Appl. No.: 18/290,214

(22) PCT Filed: May 12, 2022
(86) PCT No.: PCT/US2022/028993

§ 371 Date: Nov. 10, 2023

Related U.S. Application Data
(60) Provisional application No. 63/187,658, filed on May 12, 2021.
(60) Provisional application No. 63/238,560, filed on Aug. 30, 2021.
(60) Provisional application No. 63/245,419, filed on Sep. 17, 2021.

(51) Int. Cl. C07K 14/005; A61K 31/546; A61K 38/00; A61P 31/04

(52) U.S. Cl. CPC C07K 14/005; A61K 31/546; A61P 31/04; A61K 38/00; C12N 2740/16022; C12N 2740/16033

(57) ABSTRACT

Provided in this disclosure are formulations and methods including antimicrobial peptides and at least one antibiotic that can treat or prevent microbial films when applied on an object. Also provided in this disclosure are formulations and methods including antimicrobial peptides and at least one antibiotic that can treat or prevent microbial films when administered to a subject. Also provided in this disclosure are formulations and methods including administering antimicrobial peptides and at least one antibiotic that can improve the survivability of subjects with bacterial infections. Also provided in this disclosure are formulations and methods including administering antimicrobial peptides and at least one antibiotic that can improve the survivability of subjects with periprosthetic joint infection.

### CROSS-REFERENCE TO RELATED APPLICATIONS

This application is the United States national phase of International Patent Application No. U.S. Pat. No. 2,228,993 filed May 12, 2022, and claims priority to U.S. Provisional Application No. 63/187,658 filed May 12, 2021, U.S. Provisional Application No. 63/238,560 filed Aug. 30, 2021, and U.S. Provisional Application No. 63/245,419 filed Sep. 17, 2021, the disclosures of which are hereby incorporated by reference in their entireties.

### SEQUENCE LISTING

The Sequence Listing associated with this application is filed in electronic format via Patent Center and is hereby incorporated by reference into the specification in its entirety. The name of the text file containing the Sequence Listing is 2201879_ST25.txt. The size of the text file is 5,774 bytes, and the text file was created on Jun. 20, 2022.

### BACKGROUND

Roughly 2 million hospital associated infections occur annually in the United States. Staphylococcus aureus is a major organism responsible for these infections which include surgical site infections and implant prosthesis related infections. Total knee arthroplasty (TKA) is the largest major surgical procedure by volume in the US, with over 700,000 performed every year. An infected total knee arthroplasty, termed periprosthetic joint infection (PJI), occurs in 1.5-2% of patients undergoing joint replacement surgery. PJI treatment involves multiple surgical procedures and long-term antibiotic regimen. In acute PJI, debridement, antibiotics, and implant retention (DAIR) is a common approach for treatment. Treatment failure rate of this approach is over 60% and five-year mortality is approximately 25%. A majority of these infections are from S. aureus bacteria which are capable of forming biofilms that exhibit high antibiotic tolerance. The high antibiotic tolerance of biofilms is recognized as a primary reason for the difficulty in eradicating these infections. Novel pharmaceutical formulations and methods of treatment which have better activity against biofilms are needed. And so, there is a need for superior compositions for treatment of biofilms involving implants.

### SUMMARY

In an aspect, methods disclosed herein can be used for treating or preventing periprosthetic joint infection in a patient in need thereof, wherein a prosthetic joint is implanted in said patient; the method comprising administering: a pharmaceutical composition comprising: a peptide or pharmaceutically acceptable salt thereof, wherein said peptide has at least 70% sequence identity to a polypeptide sequence of: SA-5 (SEQ ID NO: 1); LSA-5 (SEQ ID NO: 2); WLSA-5 (SEQ ID NO: 3); LBU-1 (SEQ ID NO: 4); LBU-2 (SEQ ID NO: 5); LBU-3 (SEQ ID NO: 6); LBU-3.5 (SEQ ID NO: 7); LBU-4 (SEQ ID NO: 8); WLBU-1 (SEQ ID NO: 9); WLBU-2 (SEQ ID NO: 10); WLBU-3 (SEQ ID NO: 11); WLBU-4 (SEQ ID NO: 12); WR6 (SEQ ID NO: 13); WR12 (SEQ ID NO: 14); WR18 (SEQ ID NO: 15); or WR 24 (SEQ ID NO: 16); and an aqueous carrier; wherein said pharmaceutical composition is in a form of a liquid, wherein said pharmaceutical composition is locally administered to said prosthetic joint in vivo; and an antibiotic or pharmaceutically acceptable salt thereof; and wherein administration of said pharmaceutical composition and said antibiotic reduces a bacterial burden of said periprosthetic joint infection to a greater extent as compared to administering (i) or (ii) alone.

In some embodiments, said bacterial burden comprises implant bacterial burden, bone bacterial burden, or both. In some embodiments, said reduction of said bacterial burden is measured as colony-forming unit per milliliter (CFU/mL) by colony-forming unit (CFU) analysis. In some embodiments, said bacterial burden comprises implant bacterial burden, and wherein said method reduces said CFU/mL by at least 2.5 log. In some embodiments, said pharmaceutical composition is administered prior to said antibiotic administration. In some embodiments, said pharmaceutical composition is administered simultaneously with said antibiotic administration. In some embodiments, said pharmaceutical composition is administered subsequent to said antibiotic administration. In some embodiments, said periprosthetic joint infection further comprises a biofilm. In some embodiments, said method reduces said biofilm by at least about 10%, at least about 20%, at least about 30%, or at least about 50%. In some embodiments, said biofilm is a mature biofilm. In some embodiments, said method partially disrupts or destroys said biofilm.

In some embodiments, said locally administering comprises irrigating said prosthetic joint with said pharmaceutical composition, wherein said prosthetic joint is exposed. In some embodiments, locally administering the pharmaceutical composition to the prosthetic joint in vivo occurs for at least 5 minutes, at least 7.5 minutes, at least 15 minutes, or at least 30 minutes. In some embodiments, said locally administering the pharmaceutical composition to the prosthetic joint in vivo occurs for from about 0.1 minute to about 24 hours or about 0.1 minute to about 60 minutes. In some embodiments, said antibiotic or pharmaceutically acceptable salt thereof is administered intra-arterially, intravenously, intramuscularly, orally, subcutaneously, rectally, as inhalatory administration, or any combination thereof. In some embodiments, said antibiotic or pharmaceutically acceptable salt thereof is administered subcutaneously. In some embodiments, said peptide or pharmaceutically acceptable salt thereof is applied at least about 12 hours prior to said antibiotic or pharmaceutically acceptable salt thereof. In some embodiments, said peptide or pharmaceutically acceptable salt thereof is applied at most about 30 minutes prior to said antibiotic or pharmaceutically acceptable salt thereof. In some embodiments, said peptide or pharmaceutically acceptable salt thereof is applied at least about 12 hours subsequent to said antibiotic or pharmaceutically acceptable salt thereof. In some embodiments, said peptide or pharmaceutically acceptable salt thereof is applied at most about 30 minutes subsequent to said antibiotic or pharmaceutically acceptable salt thereof.

In some embodiments, said peptide or pharmaceutically acceptable salt is WLBU-2 (SEQ ID NO: 10). In some embodiments, said peptide or pharmaceutically acceptable is WR12 (SEQ ID NO: 14). In some embodiments, said aqueous carrier comprises phosphate buffered saline (PBS), Dulbecco's PBS, normal saline, water, lactated Ringer's solution, or aqueous sodium bicarbonate. In some embodiments, said aqueous carrier comprises Dulbecco's PBS, normal saline, water, or aqueous sodium bicarbonate. In some embodiments, said pharmaceutical composition comprises a pH value of at least about 5 to at least about 10. In some embodiments, said pharmaceutical composition comprises a pH value of at least about 7.2 to at least about 8.5. In some embodiments, said peptide or pharmaceutically acceptable salt thereof is present in said pharmaceutical composition at a concentration from at least about 0.01 μg/mL to at least about 100 mg/mL. In some embodiments, said peptide or pharmaceutically acceptable salt thereof is present in said pharmaceutical composition at a concentration from at least about 1 mg/mL to at least about 10 mg/mL. In some embodiments, said peptide or pharmaceutically acceptable salt thereof is present in said pharmaceutical composition at a concentration at about 1 mg/mL, about 3 mg/mL, or about 10 mg/mL. In some embodiments, said antibiotic is a beta-lactam antibiotic. In some embodiments, said beta-lactam antibiotic is selected from the group consisting of: Amoxillin, Ampicillin, Avibactam, Azlocillin, Aztreonam, Benzathine, Benzylpenicillin, Beta-lactam antibiotic C, Biapenem, Carbenicillin, Cefaclor, Cefadroxil, Cefamandole, Cefapirin, Cefazolin, Cefdinir, Cefditoren, Cefepime, Cefiderocol, Cefixime, Cefoperazone, Cefotetan, Cefotaxime, Cefoxitin, Cefpirome, Cefpodoxime, Cefprozil, Ceftriaxone, Ceftaroline, Ceftazidime, Ceftibuten, Ceftizoxime, Cefuroxime, Cephalexin, Cephalothin, Cephradine, Clavulanic acid, Cloxacillin, Dicloxacillin, Doripenem, Ertapenem, Faropenem, Flucloxacillin, Imipenem, Loracarbef, Mecillinam, Meropenem, Methicillin, Mezlocillin, Nafcillin, Nocardicin A, Oxacillin, Panipenem, Pheneticillin, Phenoxymethylpenicillin, Piperacillin, Procaine penicillin, Razupenem, Sulbactam, Tabtoxinine beta-lactam, Tazobactam, Tebipenem, Temocillin, Ticarillin, Thienamycin, Ticarcillin, Tigermonam, any salts thereof, and any combination thereof. In some embodiments, said beta-lactam antibiotic is Cefazolin. In some embodiments, said antibiotic is selected from the group consisting of: Amikacin, Ampicillin, Avibactim, Azithromycin, Aztreonam, Cefepime, Cefpodoxime, Ceftazidime, Ceftriaxone, Ciprofloxacin, Colistin, Daptomycin, Doxycycline, Eravacycline, Gentamicin, Levofloxacin, Linezolid, Meropenem, Penicillin G, Piperacillin, Plazomicin, Sulbactam, Tazobactam, Tetracycline, Tobramycin, Vaborbactam, Vancomycin, any salts thereof, and any combination thereof. In some embodiments, said antibiotic or pharmaceutically acceptable salt thereof is administered at a concentration of at least about 0.01 μg/mL to at least about 100 mg/mL. In some embodiments, said antibiotic or pharmaceutically acceptable salt thereof is administered at a concentration of at least about 1 mg/mL to at least about 10 mg/mL. In some embodiments, said antibiotic or pharmaceutically acceptable salt thereof is administered at a concentration of at least about 0.01 μg/kg to at least about 100 mg/kg. In some embodiments, said antibiotic or pharmaceutically acceptable salt thereof is administered at a concentration of at least about 1 mg/mL to at least about 10 mg/mL.

In some embodiments, said bacterial burden comprises a bacterial species is selected from the group consisting of Staphylococcus aureus, Staphylococcus epidermidis, Staphylococcus lugdenensis, Staphylococcus haemolyticus, Staphylococcus hominis, Staphylococcus saprophyticus, Staphylococcus simulans, Staphylococcus warnerii, Staphylococcus capitis, Staphylococcus caprae, Staphylococcus pettenkoferi, Streptococcus pyogenes, Streptococcus agalactiae, Streptococcus pneumoniae, Group C streptococci, Streptococcus constellatus, Enterococcus faecalis, Enterococcus faecium, Corynebacterium jeikeium, Lactobacillus acidophilus, Listeria monocytogenes, Escherichia coli, Klebsiella pneumoniae, Klebsiella oxytoca, Acinetobacter baumannii, Acinetobacter nosocomialis, Acinetobacter pittii, Acinetobacter haemolyticus, Acinetobacter radioresistens, Acinetobacter ursingii, Pseudomonas aeruginosa, Enterobacter cloacae, Enterobacter aerogenes, Stenotrophomonas maltophilia, Citrobacter freundii, Citrobacter koseri, Citrobacter sedlakii, Citrobacter braakii, Morganella morganii, Providencia rettgeri, Providencia stuartii, Salmonella typhimurium, Shigella dysenteriae, Moraxella catarrhalis, Neisseria gonorrhoeae, Propionibacterium acnes, Clostridioides difficile, Clostridioides perfringens, Bacteroides fragilis, Prevotella bivia, Eggerthella lenta, Peptostreptococcus anaerobius, Haemophilus parainfluenzae, Staphylococcus haemolyticus, Streptococcus dysgalactiae, and any combination thereof. In some embodiments, said bacterial species is resistant to at least one antibiotic. In some embodiments, said at least one antibiotic comprises Ampicillin, Bactrum, Clindamycin, Colistin, Erythromycin, Gentamycin, Imipenem, Levofloxacin, Linezolid, Oxacillin, Rifampin, Sulbactam, Trimethoprim, Tigecycline, Tetracycline, Vancomycin, or a combination thereof. The method of claim 1, wherein said bacterial burden comprises a bacterial selected from the group consisting of Enterococcus species, Staphylococcus aureus, Klebsiella pneumoniae, Acinetobacter species, Pseudomonas species, Enterococcus species, and any combination thereof.

In some embodiments, said methods described herein can further comprise debriding said prosthetic joint prior to administration of said pharmaceutical composition. In some embodiments, said prosthetic joint comprises replacement knee joint, replace hip joint, or replacement shoulder joint. In some embodiments, said methods described herein can further comprise reducing erythrocyte sedimentation rate (ESR) in said subject to a greater extent as compared to administering (i) or (ii) alone. In some embodiments, said ESR is measured by Westergren method. In some embodiments, said ESR is measured by Wintrobe method. In some embodiments, said methods described herein can further comprise reducing C-reactive protein expression levels in said subject a greater extent as compared to administering (i) or (ii) alone.

In some embodiments, said methods described herein can further comprise increasing a survival rate of said subject. In some embodiments, said pharmaceutical composition is administered to said subject more than once per day. In some embodiments, said antibiotic or pharmaceutically acceptable salt thereof is administered to said subject more than once per day. In some embodiments, said antibiotic or pharmaceutically acceptable salt thereof is administered to said subject more than twice per day.

In an aspect, pharmaceutical compositions described herein can comprise: a peptide or pharmaceutically acceptable salt thereof, wherein said peptide or pharmaceutically acceptable salt thereof has at least 70% sequence identity to a polypeptide sequence of SA-5 (SEQ ID NO: 1); LSA-5 (SEQ ID NO: 2); WLSA-5 (SEQ ID NO: 3); LBU-1 (SEQ ID NO: 4); LBU-2 (SEQ ID NO: 5); LBU-3 (SEQ ID NO: 6); LBU-3.5 (SEQ ID NO: 7); LBU-4 (SEQ ID NO: 8); WLBU-1 (SEQ ID NO: 9); WLBU-2 (SEQ ID NO: 10); WLBU-3 (SEQ ID NO: 11); WLBU-4 (SEQ ID NO: 12); WR6 (SEQ ID NO: 13); WR12 (SEQ ID NO: 14); WR18 (SEQ ID NO: 15); or WR 24 (SEQ ID NO: 16); an aqueous carrier; and an antibiotic or pharmaceutically acceptable salt thereof; wherein said pharmaceutical composition is in a form of a liquid.

In some embodiments, said peptide or pharmaceutically acceptable salt is WLBU-2 (SEQ ID NO: 10). In some embodiments, said peptide or pharmaceutically acceptable salt is WR12 (SEQ ID NO: 14). In some embodiments, said aqueous carrier comprises phosphate buffered saline (PBS), Dulbecco's PBS, normal saline, water, lactated Ringer's solution, or aqueous sodium bicarbonate. In some embodiments, the aqueous carrier comprises Dulbecco's PBS, normal saline, water, or aqueous sodium bicarbonate.

In some embodiments, said pharmaceutical composition comprises a pH value of at least about 5 to at least about 10. In some embodiments, said pharmaceutical composition comprises a pH value of at least about 7.2 to at least about 8.5. In some embodiments, said peptide or pharmaceutically acceptable salt thereof is present in said pharmaceutical composition at a concentration from at least about 0.01 μg/mL to at least about 100 mg/mL. In some embodiments, said peptide or pharmaceutically acceptable salt thereof is present in said pharmaceutical composition at a concentration from at least about 1 mg/mL to at least about 10 mg/mL. In some embodiments, said peptide or pharmaceutically acceptable salt thereof is present in said pharmaceutical composition at a concentration at about 1 mg/mL, about 3 mg/mL, or about 10 mg/mL. In some embodiments, said antibiotic is a beta-lactam antibiotic. In some embodiments, said beta-lactam antibiotic is selected from the group consisting of Amoxillin, Ampicillin, Avibactam, Azlocillin, Aztreonam, Benzathine, Benzylpenicillin, Beta-lactam antibiotic C, Biapenem, Carbenicillin, Cefaclor, Cefadroxil, Cefamandole, Cefapirin, Cefazolin, Cefdinir, Cefditoren, Cefepime, Cefiderocol, Cefixime, Cefoperazone, Cefotetan, Cefotaxime, Cefoxitin, Cefpirome, Cefpodoxime, Cefprozil, Ceftriaxone, Ceftaroline, Ceftazidime, Ceftibuten, Ceftizoxime, Cefuroxime, Cephalexin, Cephalothin, Cephradine, Clavulanic acid, Cloxacillin, Dicloxacillin, Doripenem, Ertapenem, Faropenem, Flucloxacillin, Imipenem, Loracarbef, Mecillinam, Meropenem, Methicillin, Mezlocillin, Nafcillin, Nocardicin A, Oxacillin, Panipenem, Pheneticillin, Phenoxymethylpenicillin, Piperacillin, Procaine penicillin, Razupenem, Sulbactam, Tabtoxinine beta-lactam, Tazobactam, Tebipenem, Temocillin, Ticarillin, Thienamycin, Ticarcillin, Tigermonam, any salts thereof, and any combination thereof. In some embodiments, said beta-lactam antibiotic is Cefazolin. In some embodiments, said antibiotic is selected from the group consisting of: Amikacin, Ampicillin, Avibactim, Azithromycin, Aztreonam, Cefepime, Cefpodoxime, Ceftazidime, Ceftriaxone, Ciprofloxacin, Colistin, Daptomycin, Doxycycline, Eravacycline, Gentamicin, Levofloxacin, Linezolid, Meropenem, Penicillin G, Piperacillin, Plazomicin, Sulbactam, Tazobactam, Tetracycline, Tobramycin, Vaborbactam, Vancomycin, any salts thereof, and any combination thereof. In some embodiments, said antibiotic or pharmaceutically acceptable salt thereof is present in said pharmaceutical composition at a concentration from at least about 0.01 μg/mL to at least about 100 mg/mL. In some embodiments, said antibiotic or pharmaceutically acceptable salt thereof is present in said pharmaceutical composition at a concentration from at least about 1 mg/mL to at least about 10 mg/mL.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an ex vivo bacterial burden analysis on implants; the implants removed from Group 2 animals treated with WLBU-2 has a decreased bacterial burden as shown compared to untreated Group 1 animals.

FIG. 2 shows an ex vivo bacterial burden analysis on bones; there is no significant difference between the analysis from bones from Group 2 animals treated with WLBU-2 and untreated Group 1 animals.

FIG. 3 shows an ex vivo total bacterial burden analysis with both implants and bones; Group 2 animals treated with WLBU-2 has a decreased bacterial burden as shown compared to untreated Group 1 animals.

FIG. 4 shows an in vivo bacterial burden analysis on implants; the implants removed from Groups 2-5 animals treated with WLBU-2 has a decreased bacterial burden as shown compared to untreated Group 1 animals.

FIG. 5 shows an in vivo bacterial burden analysis on bones; the implants removed from Groups 3-5 animals treated with WLBU-2 has a decreased bacterial burden as shown compared to untreated Group 1 animals; there is no significant difference between the analysis from the bone from Group 2 animals treated with WLBU-2 and untreated Group 1 animals.

FIG. 6 shows an in vivo total bacterial burden analysis on both implants and bones; Groups 3 & 4 animals treated with WLBU-2 has a decreased bacterial burden compared to untreated Group 1 animals; there is no significant difference between Groups 2 & 5 animals treated with WLBU-2 and untreated Group 1 animals.

FIG. 7 shows survival study body weight as a function of the number of days post infection; Group 4 animals treated with WLBU-2 and cefazolin has lower mean body weight compared to other Groups.

FIG. 8 shows survival study body weight change as a function of the number of days post-infection; there are significant differences between Group 4 animals treated with WLBU-2 and cefazolin versus untreated Group 1 animals, and between Group 4 animals versus Group 3 animals treated with WLBU-2.

FIG. 9 shows body temperature as a function of the number of days post-infection; there are no significant differences between treatment Groups 2-4 versus untreated Group 1.

FIG. 10 shows body temperature change as a function of the number of days post-infection; there are no significant differences between treatment Groups 2-4 versus untreated Group 1.

FIG. 11 shows a bacterial burden analysis on implants from the survival study; Group 2 treated with cefazolin and Group 4 treated with WLBU-2 and cefazolin has significantly lower bacterial burden than untreated Group 1.

FIG. 12 shows a bacterial burden analysis on bones from the survival study; Group 2 treated with cefazolin and Group 4 treated with WLBU-2 and cefazolin has significantly lower bacterial burden than untreated Group 1.

FIG. 13 shows a bacterial burden analysis on both implants and bones from the survival study; Group 2 treated with cefazolin and Group 4 treated with WLBU-2 and cefazolin has significantly lower bacterial burden than untreated Group 1.

FIG. 14 shows erythrocyte sedimentation rate (ESR) as a function of the number of days post-infection; treatment Groups 2-4 has significantly lower ESR than untreated Group 1.

FIG. 15 shows C-reactive protein as a function of the number of days post-infection; there are no significant differences between the Groups.

FIG. 16 shows survival rates as a function of the number of days post-infection; treatment Group 4 with WLBU-2 and cefazolin has the greatest survival rates compared to other Groups; treatment Group 2 with cefazolin has the second-best survival rates compared other groups.

FIGS. 17A-17D shows WLBU-2 (SEQ ID: 10) elimination of ESKAPE biofilms.

### DETAILED DESCRIPTION

### Pharmaceutical Composition

### Peptides

A microbial film is a community of microorganisms that live together in a layer of protective goo. The community of microorganisms may comprise a single type of microorganisms, such as exclusively S. aureus bacteria, or may comprise a collection of microorganisms, including but not limited to species of bacteria, fungus, archaea, or algae. The protective goo may be a sticky substance that adheres to surfaces, and it may be comprised of substances excreted by the microorganisms which may include, but is not limited to, excretions of polysaccharides, proteins, lipids, DNA, or any combinations thereof. Microbial films may develop on a variety of object surfaces, including biological surfaces such as the surfaces of human organs and including surfaces of inanimate object such as surfaces of medical implant devices.

In some situations, it may be desirable to treat an object surface so that the treatment prevents the growth of a microbial film on the surface. In other situations, it may be desirable to treat a microbial film on an object surface so that the treatment reduces some or all of the microbial film on the surface.

The antimicrobial peptides, such as the antimicrobial peptides (derived from lentivirus lytic peptide (LLP-1) as described herein, including WLBU-2, may be formulated or compounded into, e.g., dissolved into or otherwise dispersed into, a liquid drug product with any suitable, e.g., pharmaceutically-acceptable, aqueous solution, carrier, or excipient (collectively, aqueous carrier), such as, without limitation: water; buffer solutions; salt solutions, such as saline; buffered salt solutions, such as phosphate-buffered saline; among others as are known in the pharmaceutical and compounding arts.

The pharmaceutical composition comprising a peptide described herein, may be formulated with at least one beta lactam antibiotic to reduce bacterial burden, microbial load, or a biofilm on an object, including but not limited to: surface of a wound, an implant, a transplant, or any other object as provided in this disclosure.

A therapeutic agent is any compound or composition that is delivered to a patient to achieve a desired effect, such as a health benefit, treatment of a condition, or a curative effect.

Therapeutic agents include proteins, such as polypeptides or proteins. In the context of the present disclosure, therapeutic agents are peptides having antimicrobial activity (“antimicrobial peptides”). The antimicrobial peptides may be derived from, and are analogs of, the LLP-1 peptide parent sequence corresponding to amino acids 828-856 of the HIV-1 viral isolate HXB2R Env, including SA-5 (SEQ ID NO: 1), LSA-5 (SEQ ID NO: 2) and WLSA-5 (SEQ ID NO: 3) (see Table 1 below). The antimicrobial activity of other LLP-1 peptide analogues has been previously described (see, Tencza et al., 1999, Journal of Antimicrobial Chemotherapy 44:33-41, U.S. Pat. No. 5,714,577 of Montelaro et al. and U.S. Pat. No. 5,945,507 of Montelaro et al., the disclosures of which are incorporated herein by reference). The antimicrobial peptides may be LLP-1 analogs having modifications based on the following principles: (i) optimizing amphipathicity, (ii) substituting arginine (Arg) on the charged face and/or valine (Val) or tryptophan (Trp) on the hydrophobic face with another amino acid, and (iii) increasing peptide length, such as, without limitation LBU-1 (SEQ ID NO: 4); LBU-2 (SEQ ID NO: 5); LBU-3 (SEQ ID NO: 6); LBU-3.5 (SEQ ID NO: 7); LBU-4 (SEQ ID NO: 8); WLBU-1 (SEQ ID NO: 9); WLBU-2 (SEQ ID NO: 10); WLBU-3 (SEQ ID NO: 11); or WLBU-4 (SEQ ID NO: 12); see Table 1). Amino acid sequences are provided, left-to-right, from their N-terminus to their C-terminus.

TABLE 1
List of Exemplary Engineered Antimicrobial Peptides

| | | |
|---|---|---|
| SA-5: | RVIRV VQRAC RAIRH IVRRI RQGLR RIL | (SEQ ID |
| | Arg-Val-Ile-Arg-Val-Val-Gln-Arg-Ala-Cys-Arg-Ala-Ile-Arg- | NO: 1) |
| | His-Ile-Val-Arg-Arg-Ile-Arg-Gln-Gly-Leu-Arg-Arg-Ile-Leu | |
| LSA-5: | RVIRV VQRAC RAIRH IVRRI RQGLR RILRV V | (SEQ ID |
| | Arg-Val-Ile-Arg-Val-Val-Gln-Arg-Ala-Cys-Arg-Ala-Ile-Arg- | NO: 2) |
| | His-Ile-Val-Arg-Arg-Ile-Arg-Gln-Gly-Leu-Arg-Arg-Ile-Leu- | |
| | Arg-Val-Val | |
| WLSA-5: | RWIRV VQRWC RAIRH IWRRI RQGLR RWLRV V | (SEQ ID |
| | Arg-Trp-Ile-Arg-Val-Val-Gln-Arg-Trp-Cys-Arg-Ala-Ile-Arg- | NO: 3) |
| | His-Ile-Trp-Arg-Arg-Ile-Arg-Gln-Gly-Leu-Arg-Arg-Trp-Leu- | |
| | Arg-Val-Val | |
| LBU-1: | RVVRV VRRVV RR | (SEQ ID |
| | Arg-Val-Val-Arg-Val-Val-Arg-Arg-Val-Val-Arg-Arg | NO: 4) |
| LBU-2: | RRVVR RVRRV VRRVV RVVRR VVRR | (SEQ ID |
| | Arg-Arg-Val-Val-Arg-Arg-Val-Arg-Arg-Val-Val-Arg-Arg- | NO: 5) |
| | Val-Val-Arg-Val-Val-Arg-Arg-Val-Val-Arg-Arg | |
| LBU-3: | VRRVV RRVVR VVRRV VRRVR RVVRR VVRVV | (SEQ ID |
| | RRVVRR | NO: 6) |
| | Val-Arg-Arg-Val-Val-Arg-Arg-Val-Val-Arg-Val-Val-Arg- | |
| | Arg-Val-Val-Arg-Arg-Val-Arg-Arg-Val-Val-Arg-Arg-Val- | |
| | Val-Arg-Val-Val-Arg-Arg-Val-Val-Arg-Arg | |
| LBU-3.5: | RRVVR RVRRV VRRVV RVVRR VVRRV RRVVR | (SEQ ID |
| | RVVRV VRRVV RR | NO: 7) |
| | Arg-Arg-Val-Val-Arg-Arg-Val-Arg-Arg-Val-Val-Arg-Arg- | |
| | Val-Val-Arg-Val-Val-Arg-Arg-Val-Val-Arg-Arg-Val-Arg- | |
| | Arg-Val-Val-Arg-Arg-Val-Val-Arg-Val-Val-Arg-Arg-Val- | |
| | Val-Arg-Arg | |
| LBU-4: | RVVRV VRRVV RRVRR VVRRV VRVVR RVVRR | (SEQ ID |
| | VRRVV RRVVR VVRRV VRR | NO: 8) |
| | Arg-Val-Val-Arg-Val-Val-Arg-Arg-Val-Val-Arg-Arg-Val- | |
| | Arg-Arg-Val-Val-Arg-Arg-Val-Val-Arg-Val-Val-Arg-Arg- | |
| | Val-Val-Arg-Arg-Val-Arg-Arg-Val-Val-Arg-Arg-Val-Val- | |
| | Arg-Val-Val-Arg-Arg-Val-Val-Arg-Arg | |
| WLBU-1 | RVVRV VRRWV RR | (SEQ ID |
| | Arg-Val-Val-Arg-Val-Val-Arg-Arg-Trp-Val-Arg-Arg | NO: 9) |
| WLBU-2 | RRWVR RVRRV WRRVV RVVRR WVRR | (SEQ ID |
| | Arg-Arg-Trp-Val-Arg-Arg-Val-Arg-Arg-Val-Trp-Arg-Arg- | NO: 10) |
| | Val-Val-Arg-Val-Val-Arg-Arg-Trp-Val-Arg-Arg | |
| WLBU-3 | VRRVW RRVVR VVRRW VRRVR RVWRR VVRVV | (SEQ ID |
| | RRWVR R | NO: 11) |
| | Val-Arg-Arg-Val-Trp-Arg-Arg-Val-Val-Arg-Val-Val-Arg- | |
| | Arg-Trp-Val-Arg-Arg-Val-Arg-Arg-Val-Trp-Arg-Arg-Val- | |
| | Val-Arg-Val-Val-Arg-Arg-Trp-Val-Arg-Arg | |
| WLBU-4 | RVVRV VRRWV RRVRR VWRRV VRVVR RWVRR | (SEQ ID |
| | VRRVW RRVVR VVRRW RVV | NO: 12) |
| | Arg-Val-Val-Arg-Val-Val-Arg-Arg-Trp-Val-Arg-Arg-Val- | |
| | Arg-Arg-Val-Trp-Arg-Arg-Val-Val-Arg-Val-Val-Arg-Arg- | |
| | Trp-Val-Arg-Arg-Val-Arg-Arg-Val-Trp-Arg-Arg-Val-Val- | |
| | Arg-Val-Val-Arg-Arg-Trp-Arg-Val-Val | |

In some embodiments, the pharmaceutical composition comprises at least one peptide described herein as listed in Table 1 and/or Table 2. In some embodiments, the pharmaceutical composition comprises one or more peptides described herein as listed in Table 1 and/or Table 2. In some embodiments, the pharmaceutical composition comprises two or more peptides described herein as listed in Table 1 and/or Table 2. In some embodiments, the pharmaceutical composition comprises three or more peptides described herein as listed in Table 1 and/or Table 2. In some embodiments, the pharmaceutical composition comprises four or more peptides described herein as listed in Table 1 and/or Table 2. In some embodiments, the pharmaceutical composition comprises five or more peptides described herein as listed in Table 1 and/or Table 2. In some embodiments, the pharmaceutical composition comprises six or more peptides described herein as listed in Table 1 and/or Table 2. In some embodiments, the pharmaceutical composition comprises seven or more peptides described herein as listed in Table 1 and/or Table 2. In some embodiments, the pharmaceutical composition comprises eight or more peptides described herein as listed in Table 1 and/or Table 2. In some embodiments, the pharmaceutical composition comprises nine or more peptides described herein as listed in Table 1 and/or Table 2. In some embodiments, the pharmaceutical composition comprises ten or more peptides described herein as listed in Table 1 and/or Table 2. In some embodiments, the pharmaceutical composition comprises eleven or more peptides described herein as listed in Table 1 and/or Table 2. In some embodiments, the pharmaceutical composition comprises twelve or more peptides described herein as listed in Table 1 and/or Table 2. In some embodiments, the pharmaceutical composition comprises thirteen or more peptides described herein as listed in Table 1 and/or Table 2. In some embodiments, the pharmaceutical composition comprises fourteen or more peptides described herein as listed in Table 1 and/or Table 2. In some embodiments, the pharmaceutical composition comprises fifteen or more peptides described herein as listed in Table 1 and/or Table 2.

In some embodiments, the peptide or pharmaceutically acceptable salt thereof as described herein comprises SA-5 (SEQ ID NO: 1). In some embodiments, the peptide or pharmaceutically acceptable salt thereof as described herein comprises LSA-5 (SEQ ID NO: 2). In some embodiments, the pharmaceutical composition comprises a peptide or pharmaceutically acceptable salt thereof as described herein comprises WLSA-5 (SEQ ID NO: 3). In some embodiments, the pharmaceutical composition comprises a peptide or pharmaceutically acceptable salt thereof as described herein comprises LBU-1 (SEQ ID NO: 4). In some embodiments, the pharmaceutical composition comprises a peptide or pharmaceutically acceptable salt thereof as described herein comprises LBU-2 (SEQ ID NO: 5). In some embodiments, the peptide or pharmaceutically acceptable salt thereof as described herein comprises LBU-3 (SEQ ID NO: 6). In some embodiments, the peptide or pharmaceutically acceptable salt thereof as described herein comprises LBU-3.5 (SEQ ID NO: 7). In some embodiments, the peptide or pharmaceutically acceptable salt thereof as described herein comprises LBU-4 (SEQ ID NO: 8). In some embodiments, the peptide or pharmaceutically acceptable salt thereof as described herein comprises WLBU-1 (SEQ ID NO: 9). In some embodiments, the peptide or pharmaceutically acceptable salt thereof as described herein comprises WLBU-2 (SEQ ID NO: 10). In some embodiments, the peptide or pharmaceutically acceptable salt thereof as described herein comprises WLBU-3 (SEQ ID NO: 11). In some embodiments, the peptide or pharmaceutically acceptable salt thereof as described herein comprises WLBU-4 (SEQ ID NO: 12). In some embodiments, the peptide or pharmaceutically acceptable salt thereof as described herein comprises WR-6 (SEQ ID NO: 13). In some embodiments, the peptide or pharmaceutically acceptable salt thereof as described herein comprises WR-12 (SEQ ID NO: 14). In some embodiments, the peptide or pharmaceutically acceptable salt thereof as described herein comprises WR-18 (SEQ ID NO: 15). In some embodiments, the peptide or pharmaceutically acceptable salt thereof as described herein comprises WR-24 (SEQ ID NO: 16).

In some embodiments, the peptide or pharmaceutically acceptable salt thereof has at least 70% sequence identify to a polypeptide sequence of SA-5 (SEQ ID NO: 1), at least 70% sequence identify to a polypeptide sequence of LSA-5 (SEQ ID NO: 2), at least 70% sequence identify to a polypeptide sequence of WLSA-5 (SEQ ID NO: 3), at least 70% sequence identify to a polypeptide sequence of LBU-1 (SEQ ID NO: 4), at least 70% sequence identify to a polypeptide sequence of LBU-2 (SEQ ID NO: 5), at least 70% sequence identify to a polypeptide sequence of LBU-3 (SEQ ID NO: 6), at least 70% sequence identify to a polypeptide sequence of LBU-3.5 (SEQ ID NO: 7), at least 70% sequence identify to a polypeptide sequence of LBU-4 (SEQ ID NO: 8), at least 70% sequence identify to a polypeptide sequence of WLBU-1 (SEQ ID NO: 9), at least 70% sequence identify to a polypeptide sequence of WLBU-2 (SEQ ID NO: 10), at least 70% sequence identify to a polypeptide sequence of WLBU-3 (SEQ ID NO: 11), at least 70% sequence identify to a polypeptide sequence of WLBU-4 (SEQ ID NO: 12), at least 70% sequence identify to a polypeptide sequence of WR6 (SEQ ID NO: 13), at least 70% sequence identify to a polypeptide sequence of WR12 (SEQ ID NO: 14), at least 70% sequence identify to a polypeptide sequence of WR18 (SEQ ID NO: 15), or at least 70% sequence identify to a polypeptide sequence of WR 24 (SEQ ID NO: 16). In some embodiments, the peptide or pharmaceutically acceptable salt thereof has at least 70%, 75%, 80%, 85%, 90%, 95%, 96%, 97%, 98%, or 99% sequence identify to a polypeptide sequence listed in Table 1 and/or Table 2 and any increments of percentage therebetween.

The pharmaceutical composition and at least one beta lactam antibiotic disclosed herein below may be formulated into a drug product, e.g., wash, irrigation, spray, aerosol, or lavage liquids or solutions, and may be used, in a surgical setting and, unlike prior irrigation solutions, may be used intraoperatively, meaning the drug product may be used during a single surgical procedure without significant delay or inactive waiting periods. That is, the drug product may effectively reduce microbial load or a biofilm in a wound, e.g., decrease in microbe load, in 30 minutes or less, 20 minutes or less, 15 minutes or less, 10 minutes or less, or 5 minutes or less of contact time with a biofilm or wound. The pharmaceutical composition described herein, for example comprising a described antimicrobial peptide, and, in some instances, further comprise a physiological pH, may achieve such rapid reductions of microbial load or biofilm when used for wound wash, irrigation, or lavage.

TABLE 2
List of Exemplary Engineered
Antimicrobial Peptides

| Peptide | Sequence | Comments |
|---|---|---|
| WR6 | RRWWRR | SEQ ID |
| | Arg-Arg-Trp-Trp-Arg-Arg | NO: 13 |
| WR12 | RWWRWWRRWWRR | SEQ ID |
| | Arg-Trp-Trp-Arg-Trp-Trp- | NO: 14 |
| | Arg-Arg-Trp-Trp-Arg-Arg | |
| WR18 | WRRWWRRWWRWWRRWWRR | SEQ ID |
| | Trp-Arg-Arg-Trp-Trp-Arg- | NO: 15 |
| | Arg-Trp-Trp-Arg-Trp-Trp- | |
| | Arg-Arg-Trp-Trp-Arg-Arg | |
| WR24 | RRWWRRWRRWWRRWWRWWRRWWRR | SEQ ID |
| | Arg-Arg-Trp-Trp-Arg-Arg- | NO: 16 |
| | Trp-Arg-Arg-Trp-Trp-Arg- | |
| | Arg-Trp-Trp-Arg-Trp-Trp- | |
| | Arg-Arg-Trp-Trp-Arg-Arg | |

The antimicrobial peptides described herein are highly inhibitory to microorganisms under physiological salt concentrations and other conditions. The antimicrobial peptides function in the presence of synovial fluid and may demonstrate minimal toxicity in animal models. As a result, the antimicrobial agents may be defined as selective antimicrobial agents. The antimicrobial peptides include arginine/tryptophan-rich peptides as presented in Table 2. The peptides of SEQ ID NOs: 1-16 are described in U.S. Pat. No. 8,071,540, and their broad-spectrum antimicrobial activity is demonstrated therein and in subsequent publications.

### Antibiotics

Provided herein are antibiotics used to formulate pharmaceutical compositions described herein. In some embodiments, an antibiotic or pharmaceutically acceptable salt thereof may include but are not limited to Amikacin, Ampicillin, Avibactim, Azithromycin, Aztreonam, Cefepime, Cefpodoxime, Ceftazidime, Ceftriaxone, Ciprofloxacin, Colistin, Daptomycin, Doxycycline, Eravacycline, Gentamicin, Levofloxacin, Linezolid, Meropenem, Penicillin G, Piperacillin, Plazomicin, Sulbactam, Tazobactam, Tetracycline, Tobramycin, Vaborbactam, Vancomycin, any salts thereof, and any combination thereof. In some embodiments, the antibiotic is a beta lactam antibiotic.

### Beta Lactam Antibiotics

Provided herein are beta lactam antibiotics used to formulate pharmaceutical compositions described herein. In some embodiments, a beta lactam antibiotic or pharmaceutically acceptable salt thereof may include but are not limited to Cephalexin, Cephradine, Cephalexin, Cefadroxil, Cephalexin, Cefazolin, B-lactam antibiotic C, Cephalothin, Cefapirin, Cefuroxime, Cefprozil, Loracarbef, Cefuroxime, Cefoxitin, Cefotetan, Cefaclor, Cefamandole, Ceftriaxone, Cefdinir, Cefixime, Cefpodoxime, Cefditoren, Ceftibuten, Ceftazidime, Cefotaxime, Cefoperazone, Ceftizoxime, Cefepime, Cefiderocol, Cefpirome, Ceftaroline, Benzathine, Benzylpenicillin, Phenoxymethylpenicillin, Procaine penicillin, Pheneticillin, Cloxacillin, Dicloxacillin, Flucloxacillin, Methicillin, Nafcillin, Oxacillin, Temocillin, Amoxillin, Ampicillin, Mecillinam, Piperacillin, Carbenicillin, Ticarillin, Carbenicillin, Ticarcillin, Azlocillin, Mezlocillin, Piperacillin, Biapenem, Doripenem, Ertapenem, Faropenem, Imipenem, Meropenem, Panipenem, Razupenem, Tebipenem, Thienamycin, Aztreonam, Tigermonam, Nocardicin A, Tabtoxinine beta-lactam, Clavulanic acid, Tazobactam, Sulbactam, or Avibactam. In some embodiments, a beta lactam antibiotic or pharmaceutically acceptable salt thereof comprises Cephalexin, Cephradine, Cephalexin, Cefadroxil, Cephalexin, Cefazolin, B-lactam antibiotic C, Cephalothin, Cefapirin, Cefuroxime, Cefprozil, Loracarbef, Cefuroxime, Cefoxitin, Cefotetan, Cefaclor, Cefamandole, Ceftriaxone, Cefdinir, Cefixime, Cefpodoxime, Cefditoren, Ceftibuten, Ceftazidime, Cefotaxime, Cefoperazone, Ceftizoxime, Cefepime, Cefiderocol, Cefpirome, or Ceftaroline. In some embodiments, a beta lactam antibiotic or pharmaceutically acceptable salt thereof is cefazolin.

In some embodiments, the pharmaceutical composition described herein comprises SA-5 (SEQ ID NO: 1) and at least one β lactam antibiotic. In some embodiments, the pharmaceutical composition described herein comprises SA-5 (SEQ ID NO: 1) and a cefazolin. In some embodiments, the pharmaceutical composition described herein comprises LSA-5 (SEQ ID NO: 2) and at least one antibiotic. In some embodiments, the pharmaceutical composition described herein comprises LSA-5 (SEQ ID NO: 2) and a cefazolin. In some embodiments, the pharmaceutical composition described herein comprises WLSA-5 (SEQ ID NO: 3) and at least one antibiotic. In some embodiments, the pharmaceutical composition described herein comprises WLSA-5 (SEQ ID NO: 3) and a cefazolin. In some embodiments, the pharmaceutical composition described herein comprises LBU-1 (SEQ ID NO: 4) and at least one antibiotic. In some embodiments, the pharmaceutical composition described herein comprises LBU-1 (SEQ ID NO: 4) and a cefazolin. In some embodiments, the pharmaceutical composition described herein comprises LBU-2 (SEQ ID NO: 5) and at least one antibiotic. In some embodiments, the pharmaceutical composition described herein comprises LBU-2 (SEQ ID NO: 5) and a cefazolin. In some embodiments, the pharmaceutical composition described herein comprises LBU-3 (SEQ ID NO: 6) and at least one antibiotic. In some embodiments, the pharmaceutical composition described herein comprises LBU-3 (SEQ ID NO: 6) and a cefazolin. In some embodiments, the pharmaceutical composition described herein comprises LBU-3.5 (SEQ ID NO: 7) and at least one antibiotic. In some embodiments, the pharmaceutical composition described herein comprises LBU-3.5 (SEQ ID NO: 7) and a cefazolin. In some embodiments, the pharmaceutical composition described herein comprises LBU-4 (SEQ ID NO: 8) and at least one antibiotic. In some embodiments, the pharmaceutical composition described herein comprises LBU-4 (SEQ ID NO: 8) and a cefazolin. In some embodiments, the pharmaceutical composition described herein comprises WLBU-1 (SEQ ID NO: 9) and at least one antibiotic. In some embodiments, the pharmaceutical composition described herein comprises WLBU-1 (SEQ ID NO: 9) and a cefazolin. In some embodiments, the pharmaceutical composition described herein comprises WLBU-2 (SEQ ID NO: 10) and at least one antibiotic. In some embodiments, the pharmaceutical composition described herein comprises WLBU-2 (SEQ ID NO: 10) and a cefazolin. In some embodiments, the pharmaceutical composition described herein comprises WLBU-2 (SEQ ID NO: 11) and at least one antibiotic. In some embodiments, the pharmaceutical composition described herein comprises WLBU-2 (SEQ ID NO: 11) and a cefazolin. In some embodiments, the pharmaceutical composition described herein comprises WLBU-3 (SEQ ID NO: 11) and at least one antibiotic. In some embodiments, the pharmaceutical composition described herein comprises WLBU-3 (SEQ ID NO: 11) and a cefazolin. In some embodiments, the pharmaceutical composition described herein comprises WLBU-4 (SEQ ID NO: 12) and at least one antibiotic. In some embodiments, the pharmaceutical composition described herein comprises WLBU-4 (SEQ ID NO: 12) and a cefazolin. In some embodiments, the pharmaceutical composition described herein comprises WR-6 (SEQ ID NO: 13) and at least one antibiotic. In some embodiments, the pharmaceutical composition described herein comprises WR-6 (SEQ ID NO: 13) and a cefazolin. In some embodiments, the pharmaceutical composition described herein comprises WR-12 (SEQ ID NO: 14) and at least one antibiotic. In some embodiments, the pharmaceutical composition described herein comprises WR-12 (SEQ ID NO: 14) and a cefazolin. In some embodiments, the pharmaceutical composition described herein comprises WR-18 (SEQ ID NO: 15) and at least one antibiotic. In some embodiments, the pharmaceutical composition described herein comprises WR-24 (SEQ ID NO: 16) and a cefazolin.

### PH Buffers

In some embodiments, pharmaceutical compositions described herein are in the form of a liquid. In some embodiments, the pharmaceutical composition described herein may further comprise a physiological pH. The pharmaceutical composition may be slightly acidic, about neutral, or slightly alkaline, e.g., ranging from pH 6.5 to pH 8, including increments therebetween, such as 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, including increments therebetween. In some embodiments, the pharmaceutical composition has a pH of about at least 7.1. In some embodiments, the pharmaceutical composition has a pH of about at least 7.3. In some embodiments, the pharmaceutical composition has a pH of about at least 7.4. In some embodiments, the pharmaceutical composition has a pH of about at least 7.5.

In some embodiments, the pharmaceutical composition comprising a peptide or pharmaceutically acceptable salt is at a pH value of at least about 7.2 to at least about 7.9, at least about 7.3 to at least about 7.5, or at least about 7.4. In some embodiments, the peptide or pharmaceutically acceptable salt further comprises a pH value of at least about 7.2 to at least about 7.3, at least about 7.3 to at least about 7.4, at least about 7.4 to at least about 7.5, at least about 7.5 to at least about 7.6, at least about 7.6 to at least about 7.7, at least about 7.7 to at least about 7.8, or at least about 7.8 to at least about 7.9. In some embodiments, the pharmaceutical composition comprising a peptide or pharmaceutically acceptable salt is at a pH value of at least about 5 to at least about 10. In some embodiments, the pharmaceutical composition comprising a peptide or pharmaceutically acceptable salt is at a pH value of at least about 7.2 to at least about 8.5.

In some embodiments, the pharmaceutical composition comprising an antibiotic or pharmaceutically acceptable salt is at a pH value of at least about 7.2 to at least about 7.9, at least about 7.3 to at least about 7.5, or at least about 7.4. In some embodiments, an antibiotic or pharmaceutically acceptable salt further comprises a pH value of at least about 7.2 to at least about 7.3, at least about 7.3 to at least about 7.4, at least about 7.4 to at least about 7.5, at least about 7.5 to at least about 7.6, at least about 7.6 to at least about 7.7, at least about 7.7 to at least about 7.8, or at least about 7.8 to at least about 7.9.

In some embodiments, the pharmaceutical composition, the pH of the pharmaceutical composition may be adjusted to have a physiological pH through the addition of a pH adjusting agent, such as ammonium hydroxide, sodium hydroxide, magnesium hydroxide, sodium carbonate, other pH adjusting agents known to those skilled in the art, or combinations thereof to the aqueous carrier. Physiological pH is from at least about 7.3 to at least about 7.4.

In some embodiments, the pH adjusting agent is sodium hydroxide. In some embodiments, the pharmaceutical composition further comprises a pH buffer or pH buffering agent. Non-limiting examples of suitable pH buffers or pH buffering agents includes sodium citrate, magnesium carbonate, magnesium bicarbonate, calcium carbonate, calcium bicarbonate, sodium bicarbonate, potassium bicarbonate, magnesium lactate, magnesium glucomate, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium carbonate, calcium acetate, tris(hydroxymethyl)aminomethane, other pH buffers known to those skilled in the art, or combinations thereof. In some embodiments, suitable pH buffers are bicarbonate, phosphate, lactate, acetate, tris(hydroxymethyl)aminomethane, or salts thereof, or any combination thereof. In some embodiments, the pH buffer is a phosphate buffer. In some embodiments, the phosphate buffer is Dulbecco's phosphate buffered saline (dPBS). In some embodiments, the pH buffer is a component of an aqueous carrier.

The pharmaceutical composition may be physiologically hypertonic, isotonic, or hypotonic. In some embodiments, the pharmaceutical composition further comprises an aqueous carrier. In some embodiments, comprises phosphate buffered saline (PBS), Dulbecco's PBS, normal saline, water, lactated Ringer's solution, or aqueous sodium bicarbonate. In some embodiments, the aqueous carrier is lactated Ringer's solution, normal saline (0.9% w/v), sterile water for injection, or aqueous sodium carbonate. In some embodiments, the aqueous carrier is lactated Ringer's solution. In some embodiments, the aqueous carrier is normal saline (0.9% w/v). In some embodiments, the aqueous carrier is sterile water for injection. In some embodiments, the aqueous carrier is aqueous sodium bicarbonate. In some embodiments, the aqueous sodium bicarbonate is 8.4% sodium bicarbonate. In some embodiments, the pharmaceutical composition comprising the aqueous carrier may be physiologically isotonic, as with lactated Ringer's solution or normal saline (0.9% w/v), or be physiologically hypotonic (sub-physiologic osmolarity or osmolality), such as modified versions of either lactated Ringer's solution or normal saline diluted, for example, with water. The physiologically hypotonic carrier may have a pH greater than 5.0, or may be alkaline, that is, having a pH of greater than 7.0. In some embodiments, the aqueous carrier has a total osmolarity ranging from about 1 milliosmoles per one liter (mOsm/L) to about 5,000 mOsm/L. In some embodiments, the aqueous carrier has a total osmolarity of about 1 mOsm/L, about 50 mOsm/L, about 100 mOsm/L, about 150 mOsm/L, about 200 mOsm/L, about 250 mOsm/L, about 300 mOsm/L, about 350 mOsm/L, about 400 mOsm/L, about 450 mOsm/L, about 500 mOsm/L, about 1000 mOsm/L, about 1500 mOsm/L, about 2000 mOsm/L, about 2500 mOsm/L, about 3000 mOsm/L, about 3500 mOsm/L, about 4000 mOsm/L, about 4500 mOsm/L, or about 5000 mOsm/L. In some embodiment, the aqueous carrier has a total osmolality ranging from about 1 milliosmole per kilogram (mOsm/kg) from 5000 mOsm/kg.

In some embodiments, the aqueous carrier has a total osmolarity of about 1 mOsm/kg, about 50 mOsm/kg, about 100 mOsm/kg, about 150 mOsm/kg, about 200 mOsm/kg, about 250 mOsm/kg, about 300 mOsm/kg, about 350 mOsm/kg, about 400 mOsm/kg, about 450 mOsm/kg, about 500 mOsm/kg, about 1000 mOsm/kg, about 1500 mOsm/kg, about 2000 mOsm/kg, about 2500 mOsm/kg, about 3000 mOsm/kg, about 3500 mOsm/kg, about 4000 mOsm/kg, about 4500 mOsm/kg, or about 5000 mOsm/kg. In some embodiments, the aqueous carrier may have a total ionic strength ranging from about 0.001 molar (M) and 1.0 M. In some embodiments, the aqueous carrier may have a total ionic strength of about 0.001 M, about 0.01 M, about 0.015 M, about 0.02 M, about 0.025 M, about 0.03 M, about 0.035 M, about 0.04 M, about 0.05 M, about 0.055 M, about 0.06 M, about 0.065 M, about 0.07 M, about 0.075 M, about 0.08 M, about 0.085 M, about 0.09 M, about 0.1 M, about 0.12 M, about 0.14 M, about 0.15 M, about 0.16 M, about 0.18 M, about 0.2 M, about 0.22 M, about 0.24 M, about 0.25 M, about 0.26 M, about 0.28 M, about 0.03 M, about 0.35 M, about 0.4 M, about 0.45 M, about 0.5 M, about 0.55 M, about 0.6 M, about 0.65 M, about 0.7 M, about 0.75 M about 0.8 M, about 0.85 M, about 0.9 M, about 0.95 M, or about 1.0 M. In some embodiments, the aqueous carrier may have an electrolyte concentration ranging from about 0.1 milliequivalent per mL (mEq/mL) to about 1000 mEq/mL. In some embodiments, the aqueous carrier may have an electrolyte concentration of about 0.1 mEq/mL, about 1 mEq/mL, about 25 mEq/mL, about 50 mEq/mL, about 100 mEq/mL, about 150 mEq/mL, about 200 mEq/mL, about 250 mEq/mL, about 300 mEq/mL, about 350 mEq/mL, about 400 mEq/mL, about 450 mEq/mL, about 500 mEq/mL, about 550 mEq/mL, about 600 mEq/mL, about 650 mEq/mL, about 700 mEq/mL, about 750 mEq/mL, about 800 mEq/mL, about 850 mEq/mL, about 900 mEq/mL, about 950 mEq/mL, or about 1000 mEq/mL. In some embodiments, the aqueous carrier may have an electrolyte concentration of about 1 mEq/mL to about 500 mEq/mL, about 50 mEq/mL to about 500 mEq/mL, about 100 mEq/mL to about 300 mEq/mL, about 125 mEq/mL to about 250 mEq/mL, about 100 mEq/mL to about 500 mEq/mL, or about 100 mEq/mL to about 1000 mEq/mL.

### Pharmaceutical Acceptable Excipients

By “pharmaceutically acceptable”, it is meant that the carrier, diluent, or excipient must be compatible with the other ingredients of the composition and not deleterious to the recipient thereof. The term “compatible”, as used herein, means that the components of the composition are capable of being commingled with the subject compound, and with each other, in a manner such that there is no interaction that would substantially reduce the pharmaceutical efficacy of the composition under ordinary use situations. In some embodiments, a pharmaceutical formulation further comprises an excipient. In some embodiments, an excipient can be a chelator. In some embodiments, a chelator can be a fungicidal chelator. In some embodiments, a pharmaceutical formulation further comprises a diluent. In some embodiments, a diluent can be an aqueous acid. In some embodiments, a pharmaceutical formulation further comprises cysteamine. In some embodiments, a pharmaceutical formulation further comprises a surfactant. In some embodiments, a pharmaceutical formulation further comprises a small molecule. In some embodiments, the pharmaceutical composition further comprises one or more additional pharmaceutically acceptable excipients. See, e.g., Remington: The Science and Practice of Pharmacy (Gennaro, 21^{st} Ed. Mack Pub. Co., Easton, PA (2005) for a list of pharmaceutically acceptable excipients. In some embodiments, the pharmaceutically acceptable excipient is of sufficiently high purity and sufficiently low toxicity to render them suitable for administration preferably to an animal, preferably a mammal, being treated.

Some other examples of substances, which may serve as pharmaceutically acceptable excipients include:
Amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, the amino acid is arginine. In some embodiments, the amino acid is L-arginine.
Monosaccharides such as glucose (dextrose), arabinose, mannitol, fructose (levulose), and galactose.
Cellulose and its derivatives such as sodium carboxymethyl cellulose, ethyl cellulose, and methyl cellulose.
Solid lubricants such as talc, stearic acid, magnesium stearate and sodium stearyl fumarate.
Polyols such as propylene glycol, glycerin, sorbitol, mannitol, and polyethylene glycol.
Emulsifiers such as the polysorbates.
Wetting agents such as sodium lauryl sulfate, Tween®, Span, alkyl sulphates, and alkyl ethoxylate sulphates.
Cationic surfactants such as cetrimide, benzalkonium chloride, and cetylpyridinium chloride.
Diluents such as calcium carbonate, microcrystalline cellulose, calcium phosphate, starch, pregelatinized starch, sodium carbonate, mannitol, and lactose.
Binders such as starches (corn starch and potato starch), gelatin, sucrose hydroxypropyl cellulose (HPC), polyvinylpyrrolidone (PVP), and hydroxypropyl methyl cellulose (HPMC).
Disintegrants such as starch, and alginic acid.
Super-disintegrants such as ac-di-sol, croscarmellose sodium, sodium starch glycolate and crospovidone.
Glidants such as silicon dioxide.
Coloring agents such as the FD&C dyes.
Sweeteners and flavoring agents, such as aspartame, saccharin, menthol, peppermint, and fruit flavors.
Preservatives such as benzalkonium chloride, PHMB, chlorobutanol, thimerosal, phenylmercuric, acetate, phenylmercuric nitrate, parabens, and sodium benzoate.
Tonicity adjustors such as sodium chloride, potassium chloride, mannitol, and glycerin.
Antioxidants such as sodium bisulfite, acetone sodium bisulfite, sodium formaldehyde, sulfoxylate, thiourea, and EDTA.
Cryoprotectants such as sodium or potassium phosphates, citric acid, tartaric acid, gelatin, and carbohydrates such as dextrose, mannitol, and dextran.
Surfactants such as sodium lauryl sulfate. For example, cationic surfactants such as cetrimide (including tetradecyl trimethyl ammonium bromide with dodecyl and hexadecyl compounds), benzalkonium chloride, and cetylpyridinium chloride. Some examples of anionic surfactants are alkylsulphates, alkylethoxylate sulphates, soaps, carxylate ions, sulfate ions, and sulfonate ions. Some examples of non-ionic surfactants are polyoxyethylene derivatives, polyoxypropylene derivatives, polyol derivatives, polyol esters, polyoxyethylene esters, poloxamers, glocol, glycerol esters, sorbitan derivatives, polyethylene glycol (such as PEG-40, PEG-50, or PEG-55) and esters of fatty alcohols.
Organic materials such as carbohydrates, modified carbohydrates, lactose (including a-lactose, monohydrate spray dried lactose or anhydrous lactose), starch, pregelatinized starch, sucrose, mannitol, sorbital, cellulose (including powdered cellulose and microcrystalline cellulose).
Inorganic materials such as calcium phosphates (including anhydrous dibasic calcium phosphate, dibasic calcium phosphate or tribasic calcium phosphate).
Co-processed diluents.
Compression aids.
Anti-tacking agents such as silicon dioxide and talc.

### Methods of Treatment

Provided herein are methods of treating or preventing periprosthetic joint infection comprises administering a pharmaceutical composition comprising a peptide described herein and an antibiotic to a patient, wherein a periprosthetic joint in implanted in the patient. In some embodiments, the pharmaceutical composition herein is administered locally to an implanted prosthetic joint in vivo. In some embodiments, administration of a pharmaceutical composition comprising a peptide described herein and an antibiotic together treats, prevents, or reduces a bacterial burden of the periprosthetic joint infection to a greater extent as compared to administering a pharmaceutical composition comprising a peptide described herein or an antibiotic alone.

Provided herein are methods for treating or preventing periprosthetic joint infection in a patient in need thereof, wherein a prosthetic joint is implanted in the patient; the method comprising administering a pharmaceutical composition comprising a peptide described herein; wherein the pharmaceutical composition is in a form of a liquid, wherein the pharmaceutical composition is locally administered to the prosthetic joint in vivo, and an antibiotic or pharmaceutically acceptable salt thereof, wherein administration of the pharmaceutical composition and the antibiotic reduces a bacterial burden of the periprosthetic joint infection to a greater extent as compared to administering the pharmaceutical composition or antibiotic alone. [0056]. In some embodiments, locally administration of the pharmaceutical composition comprising a peptide described herein onto the prosthetic joint in vivo improves reduction of the bacterial burden and allows treatment without removing the prosthetic joint from the patient. In some embodiments, locally administration of the pharmaceutical composition comprising a peptide described herein on the prosthetic joint improves reduction of the bacterial burden. In some embodiments, administration of a pharmaceutical composition comprising a peptide described herein and an antibiotic together provides a synergistic effect in decreasing a bacterial burden of the periprosthetic joint infection, improves survivability of the patient, reduces erythrocyte sedimentation rate (ESR) in a patient, reduces C-reactive protein expression levels in a patient, or any combination thereof. In some embodiments, administration of a pharmaceutical composition comprising a peptide described herein and an antibiotic together provides a synergistic effect in decreasing a bacterial burden of the periprosthetic joint infection, improves survivability of the patient, reduces erythrocyte sedimentation rate (ESR) in a patient, reduces C-reactive protein expression levels in a patient, or any combination thereof. In some embodiments, administration of a pharmaceutical composition comprising a peptide described herein and an antibiotic together decreases a bacterial burden to a greater extent as compared to administering the pharmaceutical composition or antibiotic alone. In some embodiments, administration of a pharmaceutical composition comprising a peptide described herein and an antibiotic together improves survivability of the patient to a greater extent as compared to administering the pharmaceutical composition or antibiotic alone. In some embodiments, administration of a pharmaceutical composition comprising a peptide described herein and an antibiotic together reduces erythrocyte sedimentation rate (ESR) of the patient to a greater extent as compared to administering the pharmaceutical composition or antibiotic alone. In some embodiments, administration of a pharmaceutical composition comprising a peptide described herein and an antibiotic together reduces C-reactive protein expression levels of the patient to a greater extent as compared to administering the pharmaceutical composition or antibiotic alone.

Provided herein are methods for preventing a formation of a microbial biofilm or reducing the microbial biofilm mass on an object, comprising administering effective amounts of a peptide or pharmaceutically acceptable salt thereof and at least one antibiotic or pharmaceutically acceptable salt thereof, wherein the peptide and at least one antibiotic are applied to the object for a sufficient amount of time such that the microbial biofilm is not formed or such that the mass of the microbial biofilm is reduced. In some embodiments, a microbial biofilm is a bacterial biofilm.

In some embodiments, administration of the peptide or pharmaceutically acceptable salt may be administered orally, rectally, or parenterally, in formulations containing conventionally acceptable carriers, adjuvants, and vehicles as desired. Administration can also be intra-arterial, intravenous, intramuscular, oral, subcutaneous, intranasal, inhalational, any other administrative routes known to those skilled in the art, or any combination thereof. In some embodiments, administration may be through injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal), any other method of administration known to those skilled in the art, or any combinations thereof. In some embodiments, the administration may be administered as a spray or as a wash. In some exemplary embodiments, a route of administration may be an injection such as an inter-articular intramuscular injection, intravenous injection, subcutaneous injection, intraperitoneal injection. In some exemplary embodiments, a route of administration may be via subcutaneous. In some exemplary embodiments, a route of administration may be via inter-articular. In some embodiments, prior to administration of the pharmaceutical composition described herein, a site may be incised and the bacterial infection drained, resulting in an open wound. In some embodiments, a route of administration comprises administering the pharmaceutical composition to an open wound, washing an open wound with the pharmaceutical composition, or debriding an open wound with the pharmaceutical composition. In some embodiments, the route of administration comprises washing an implanted device. In some embodiments, the route of administration comprises draining the bacterial infection and washing an implanted device. In some embodiments, the peptide or pharmaceutically acceptable salt thereof may be formulated into a drug product, e.g., wash, irrigation, or lavage liquids or solutions, and may be used, in a surgical setting and, unlike prior irrigation solutions, can be used intraoperatively, meaning they can be used during a single surgical procedure without significant delay or inactive waiting periods. In some embodiments, administration of the peptide or pharmaceutically acceptable salt occurs in vivo. In some embodiments, locally administering the peptide or pharmaceutically acceptable salt comprises irrigating a prosthetic joint or open wound. In some embodiments, locally administering the peptide or pharmaceutically acceptable salt comprises irrigating a prosthetic joint. In some embodiments, wherein the prosthetic joint is implanted and does not require removal for treatment (e.g., in vivo). In some embodiments, locally administering the peptide or pharmaceutically acceptable salt comprises irrigating a prosthetic joint, wherein the prosthetic joint is exposed.

In some embodiments, an antibiotic or pharmaceutically acceptable salt may be administered orally, rectally, or parenterally, in formulations containing conventionally acceptable carriers, adjuvants, and vehicles as desired. Administration may also be intra-arterial, intravenous, intramuscular, oral, subcutaneous, intranasal, inhalational, any other administrative routes known to those skilled in the art, or any combination thereof. In some embodiments, administration may be an injection or an infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous, inhalational, transdermal, transmucosal, sublingual, buccal and topical (including epicutaneous, dermal, enema, eye drops, ear drops, intranasal, vaginal), any other method of administration known to those skilled in the art, or any combinations thereof. In some embodiments, the administration may be administered a spray or as a wash. In some exemplary embodiments, a route of administration may be via an injection such as an inter-articular intramuscular injection, intravenous injection, subcutaneous injection, intraperitoneal injection, or any other route of injection known to those skilled in the art. In some exemplary embodiments, a route of administration may be via inhalation. In some exemplary embodiments, a route of administration may be intraocular. In some exemplary embodiments, a route of administration may be via subcutaneous. In some exemplary embodiments, a route of administration may be via inter-articular. In some embodiments, prior to administration of the pharmaceutical composition described herein, a site may be incised and the bacterial infection drained, resulting in an open wound. In some embodiments, a route of administration comprises administering the pharmaceutical composition to an open wound, washing an open wound with the pharmaceutical composition, and debriding an open would with the pharmaceutical composition. In some embodiments, the route of administration comprises washing an implanted device. In some embodiments, the route of administration comprises draining the bacterial infection and washing an implanted device. In some embodiments, an antibiotic or pharmaceutically acceptable salt may be formulated into a drug product, e.g., wash, irrigation, or lavage liquids or solutions, and may be used, in a surgical setting and, unlike prior irrigation solutions, may be used intraoperatively, meaning they can be used during a single surgical procedure without significant delay, or inactive waiting periods.

In some embodiments, the peptide disclosed herein or pharmaceutically acceptable salt thereof prevents substantially the formation of a microbial biofilm. Using suitable detection methods for detecting a microbial biofilm, a microbial biofilm is not detectable on an object when the microbial biofilm is substantially prevented to be formed after treatment of the peptide or pharmaceutically acceptable salt thereof.

In some embodiments, the peptide disclosed herein or pharmaceutically acceptable salt thereof may reduce the mass of the microbial biofilm. In some embodiments, the peptide disclosed herein or pharmaceutically acceptable salt thereof may lead to at least about 0.5%, 1%, 2%, 3%, 4%, 5%, 6%, 7%, 8%, 9%, 10%, 12.5%, 15%, 17.5%, 20%, 22.5%, 25%, 27.5%, 30%, 32.5%, 35%, 37.5%, 40%, 42.5%, 45%, 47.5%, 50%, 60%, 70%, 80%, 90%, 91%, 92%, 93%, 94%, 95%, 96%, 97%, 98%, or 99% reduction in mass and any increments of percentage therebetween. The mass of a microbial film may be measured by weighing an object before and after a biofilm has been grown on the object and then taking the difference. The dry mass of a microbial film may be measured by weighing an object before a microbial film has been grown, and after a microbial film has been grown and dried in an oven, and then taking the difference in the weight measurements. The mass of a microbial film may also be measured with any other method known to those skilled in the art.

In some embodiments, the object may be a solid object, a liquid object, a hard object, a soft object, a metallic object, a polymeric object, a ceramic object, or a composite object. For example, an object may be a surgical table in an operation room, a bathroom sink, a bathroom tube, a piece of furniture that needs frequently disinfection, etc. The examples listed herein are not meant to be limiting.

In other embodiments, the object may be a biological object, food objects, members of the plantae kingdom, members of the animal kingdom, a human being, parts of the human body, parts of an animal body, a biological transplant object, a biological implant object, a replaced joint, or any other object with a surface and combinations thereof. In some embodiments, the biological transplant object may be a kidney, liver, heart, lung, pancreas, intestine, ligament, tendon, skin, or any other body part, organ, or tissue known to those skilled in the art.

In some embodiments, the biological implant object may be a joint implant, a cochlear implant, a dental implant, an ophthalmic implant, an ocular implant, a neural implant, a cardiovascular implant, a renal implant, a urinary implant, a penile implant, a reconstructive implant, a plastic implant, a breast implant, an aqueous shunt implant, an auditory osseointegrated device implant, a hallux implant, a metacarpophalangeal implant, a vascular implant, a metatarsal joint implant, a collagen implant, a dextranomer/hyaluronic acid implant, a tracheoesophageal implant, and any other implant known to those skilled in the art.

In some embodiments of a pharmaceutical composition comprising (i) a peptide or pharmaceutically acceptable salt thereof, and (ii) an antibiotic or pharmaceutically acceptable salt thereof, administration of the pharmaceutical composition may be administered orally, rectally, or parenterally, in formulations containing conventionally acceptable carriers, adjuvants, and vehicles as desired. Administration can also be intra-arterial, intravenous, intramuscular, oral, subcutaneous, intranasal, inhalational, any other administrative routes known to those skilled in the art, or any combination thereof. In some embodiments, administration may be through injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal), any other method of administration known to those skilled in the art, or any combinations thereof. In some embodiments, the administration may be administered as a spray or as a wash. In some exemplary embodiments, a route of administration may be an injection such as an inter-articular intramuscular injection, intravenous injection, subcutaneous injection, intraperitoneal injection. In some exemplary embodiments, a route of administration may be via subcutaneous. In some exemplary embodiments, a route of administration may be via inter-articular. In some embodiments, prior to administration of the pharmaceutical composition described herein, a site may be incised and the bacterial infection drained, resulting in an open wound. In some embodiments, a route of administration comprises administering the pharmaceutical composition to an open wound, washing an open wound with the pharmaceutical composition, or debriding an open wound with the pharmaceutical composition. In some embodiments, the route of administration comprises washing an implanted device. In some embodiments, the route of administration comprises draining the bacterial infection and washing an implanted device. In some embodiments, the pharmaceutical composition may be formulated into a drug product, e.g., wash, irrigation, or lavage liquids or solutions, and may be used, in a surgical setting and, unlike prior irrigation solutions, can be used intraoperatively, meaning they can be used during a single surgical procedure without significant delay or inactive waiting periods.

Provided herein are methods for preventing a formation of a microbial biofilm or reducing said microbial biofilm mass on an object. The methods comprise administering effective amounts of a peptide or pharmaceutically acceptable salt thereof and an antibiotic or pharmaceutically acceptable salt thereof, wherein the peptide and the antibiotic are applied to the object for a sufficient amount of time such that said microbial biofilm is not formed or said mass of said microbial biofilm is reduced.

In some embodiments, sufficient time is at least about 0.5 min to at least about 600 min. In some embodiments, the sufficient time is at least about 5 min to at least about 60 min. In some embodiments, the sufficient time is at least about 7.5 min. In some embodiments, the sufficient time is at least about 15 min. In some embodiments, the sufficient time is at least about 30 min. In some embodiments, the sufficient is at least about 0.1 min, 0.5 min, 1 min, 2 min, 3 min, 4 min, 5 min, 10 min, 15 min, 20 min, 25 min, 30 min, 35 min, 40 min, 45 min, 50 min, 60 min, 2 hours, 5 hours, 10 hours, 15 hours, 20 hours, 24 hours, 48 hours, 72 hours, 4 days, 1 week, or any increments of time therebetween.

In some embodiments, the pharmaceutical composition disclosed herein may prevent the formation of a microbial biofilm. In some embodiments, the peptide disclosed herein or pharmaceutically acceptable salt thereof may reduce the mass of the microbial biofilm.

In some embodiments, the pharmaceutical composition disclosed herein for treating a microbial film may lead to at least about 10% reduction in bacterial burden, at least about 20% reduction in bacterial burden, at least about 30% reduction in bacterial burden, at least about 40% reduction in bacterial burden, at least about 50% reduction in bacterial burden, at least about 60% reduction in bacterial burden, at least about 70% reduction in bacterial burden, at least about 80% reduction in bacterial burden, or at least about 90% reduction in bacterial burden and any increments therebetween. In some embodiments, the antibiotic disclosed herein for treating a microbial film may lead to at least about 10% reduction in bacterial burden, at least about 20% reduction in bacterial burden, at least about 30% reduction in bacterial burden, at least about 40% reduction in bacterial burden, at least about 50% reduction in bacterial burden, at least about 60% reduction in bacterial burden, at least about 70% reduction in bacterial burden, at least about 80% reduction in bacterial burden, or at least about 90% reduction in bacterial burden and any increments therebetween. In some embodiments, the pharmaceutical composition comprising a peptide disclosed herein administered with the antibiotic for the method of treating a microbial film may lead to at least about 10% reduction in bacterial burden, at least about 20% reduction in bacterial burden, at least about 30% reduction in bacterial burden, at least about 40% reduction in bacterial burden, at least about 50% reduction in bacterial burden, at least about 60% reduction in bacterial burden, at least about 70% reduction in bacterial burden, at least about 80% reduction in bacterial burden, or at least about 90% reduction in bacterial burden and any increments therebetween. In some embodiments, the bacterial burden may be measured by colony forming units (CFU) analysis, which may involve taking a sample from a microbial film, diluting the sample, growing cell cultures from the sample on a Petri dish for a predetermined amount of time, and counting the number of colonies formed. In some embodiment, the pharmaceutical composition comprising a peptide disclosed herein for treating a microbial film may lead partially disrupts or destroys a microbial film. In some embodiment, the antibiotic disclosed herein for treating a microbial film may lead partially disrupts or destroys a microbial film. In some embodiment, the pharmaceutical composition disclosed herein for treating a microbial film may lead partially disrupts or destroys a microbial film

In some embodiments, the peptide or pharmaceutically acceptable salt thereof may be applied simultaneously with at least one antibiotic or pharmaceutically acceptable salt thereof. In some embodiments, the peptide or pharmaceutically acceptable salt thereof is applied prior to at least one antibiotic or pharmaceutically acceptable salt thereof. In some embodiments, the peptide or pharmaceutically acceptable salt thereof is applied subsequent to at least one antibiotic or pharmaceutically acceptable salt thereof. In some embodiments, the peptide or pharmaceutically acceptable salt thereof is applied at least about 0.1 min, 0.5 min, 1 min, 2 min, 3 min, 4 min, 5 min, 10 min, 15 min, 20 min, 25 min, 30 min, 35 min, 40 min, 45 min, 50 min, 60 min, 2 hours, 5 hours, 10 hours, 15 hours, 20 hours, 24 hours, 48 hours, 72 hours, 4 days, 1 week, or any increments of time therebetween prior to at least one antibiotic or pharmaceutically acceptable salt thereof. In some embodiments, the peptide or pharmaceutically acceptable salt thereof is applied at least about 12 hours prior to at least one antibiotic or pharmaceutically acceptable salt thereof. In some embodiments, a peptide or pharmaceutically acceptable salt thereof is applied at most about 30 minutes prior to at least one antibiotic or pharmaceutically acceptable salt thereof.

In some embodiments, the peptide or pharmaceutically acceptable salt thereof is applied at least about 0.1 min, 0.5 min, 1 min, 2 min, 3 min, 4 min, 5 min, 10 min, 15 min, 20 min, 25 min, 30 min, 35 min, 40 min, 45 min, 50 min, 60 min, 2 hours, 5 hours, 10 hours, 15 hours, 20 hours, 24 hours, 48 hours, 72 hours, 4 days, 1 week, or any increments of time therebetween subsequent to at least one antibiotic or pharmaceutically acceptable salt thereof. In some embodiments, the peptide or pharmaceutically acceptable salt thereof is applied at least about 12 hours subsequent to at least one antibiotic or pharmaceutically acceptable salt thereof. In some embodiments, the peptide or pharmaceutically acceptable salt thereof is applied at most about 30 minutes subsequent to administration of at least one antibiotic or pharmaceutically acceptable salt thereof.

In some embodiments, the method of administration may further comprise debriding. In some embodiments, the open wound, infection, or prosthetic joint is debrided prior to administration of a pharmaceutical composition described herein. In some embodiments, the prosthetic joint is debrided prior to administration of a pharmaceutical composition described herein. In some embodiments, the method of administration may further comprise washing. In some embodiments, the method of administration may further comprise incising and draining prior to administration of peptide or pharmaceutically acceptable salt. In some embodiments, the method of administration may further comprise incising and draining subsequent to administration of peptide or pharmaceutically acceptable salt. In some embodiments, the method of administration may further comprise incising and draining simultaneously with the administration of peptide or pharmaceutically acceptable salt. In some embodiments, the method of administration may further comprise incising and draining prior to administration of at least one antibiotic or pharmaceutically acceptable salt. In some embodiments, the method of administration may further comprise incising and draining subsequent to administration of at least one antibiotic or pharmaceutically acceptable salt. In some embodiments, the method of administration may further comprise incising and draining simultaneously with the administration of at least one antibiotic or pharmaceutically acceptable salt.

In some embodiments, the method of administration may last over a course of at least about 0.1 min, 0.5 min, 1 min, 2 min, 3 min, 4 min, 5 min, 10 min, 15 min, 20 min, 25 min, 30 min, 35 min, 40 min, 45 min, 50 min, 1 hour, 5 hours, 12 hours, 24 hours, 48 hours, 72 hours, 4 days, 5 days, 1 week, 2 weeks, 3 week, 4 weeks, 2 months, 3 months, 4 months, 5 months, 6 months, 7 months, 8 months, 9 months, 10 months, 11 months, 12 months, 2 years, 3 years, 4 years, 5 years, 6 years, 7 years, 8 years, 9 years, 10 years, 20 years, 25 years, 30 years, 35 years, 40 years, 45 years, 50 years, 55 years, 60 years, 65 years, 70 years, 75 years, or 80 years.

Provided herein are methods for treating or preventing a bacterial infection. The methods comprise administering a pharmaceutical composition comprising a peptide or pharmaceutically acceptable salt thereof as described herein and the pharmaceutical formulation comprises a physiological pH and administering at least one antibiotic or pharmaceutically acceptable salt.

In some embodiments, the method for treating or preventing a bacterial infection comprises administering a pharmaceutical composition comprising a peptide or pharmaceutically acceptable salt thereof as described herein and the pharmaceutical formulation comprises a physiological pH simultaneously with administering at least one antibiotic or pharmaceutically acceptable salt.

In some embodiments, the method for treating or preventing a bacterial infection comprises administering a pharmaceutical composition comprising a peptide or pharmaceutically acceptable salt thereof as described herein and the pharmaceutical formulation comprises a physiological pH prior to administering at least one antibiotic or pharmaceutically acceptable salt.

In some embodiments, the method for treating or preventing a bacterial infection comprises administering a pharmaceutical composition comprising a peptide or pharmaceutically acceptable salt thereof as described herein and the pharmaceutical formulation comprises a physiological pH subsequent to administering at least one antibiotic or pharmaceutically acceptable salt thereof.

In some embodiments, the method for treating or preventing a bacterial infection in a subject comprises administering a pharmaceutical composition comprising a peptide or pharmaceutically acceptable salt thereof as described herein and the pharmaceutical formulation comprises a physiological pH, and administering at least one antibiotic or pharmaceutically acceptable salt thereof resulting in reducing a bacterial burden of the bacterial infection by at least about 1.0 log colony-forming unit (CFU)/mL of microbes as compared to treating the bacterial infection with the peptide only or with the antibiotic only.

In some embodiments, the method for treating comprises administering the combination treatment of the peptide and the antibiotic may result in reducing more than 1.0 log CFU/mL or more than 2.5 log CFU/mL of microbes as compared to administering peptide or beta lactam antibiotic only.

In some embodiments, the method for treating comprises administering the combination treatment of a pharmaceutical composition comprising a peptide described herein and an antibiotic may result in reducing more than 1.0 log CFU/mL, more than 1.1 log CFU/mL, more than 1.2 log CFU/mL, more than 1.5 log CFU/mL, more than 2 log CFU/mL, more than 2.5 log CFU/mL, more than 3 log CFU/mL, more than 3.5 log CFU/mL, or more than 4 log CFU/mL of a bacterial burden as compared to administering peptide or antibiotic only. In some embodiments, the method for treating comprises administering the combination treatment of a pharmaceutical composition comprising a peptide described herein and an antibiotic may result in reducing at least 1.0 log CFU/mL, at least 1.1 log CFU/mL, at least 1.2 log CFU/mL, at least 1.5 log CFU/mL, at least 2 log CFU/mL, at least 2.5 log CFU/mL, at least 3 log CFU/mL, at least 3.5 log CFU/mL, or at least 4 log CFU/mL of a bacterial burden as compared to administering peptide or antibiotic only.

In some embodiments, the combination treatment of administering a pharmaceutical composition comprising a peptide or pharmaceutically acceptable salt thereof as described herein and the pharmaceutical formulation comprises a physiological pH and administering at least one antibiotic or pharmaceutically acceptable salt thereof resulting in an increased survivability of the subject as compared to treating the subject with only the peptide or the antibiotic. In some embodiments, the method for treating or preventing a bacterial infection in a subject comprises administering a pharmaceutical composition comprising a peptide or pharmaceutically acceptable salt thereof as described herein and the pharmaceutical formulation comprises a physiological pH, and administering at least one antibiotic or pharmaceutically acceptable salt thereof resulting in an increased survivability of the subject as compared to administering to irrigation and debridement (I&D).

In some embodiments, the method for treating or preventing a bacterial infection in a subject comprises administering a pharmaceutical composition comprising a peptide or pharmaceutically acceptable salt thereof as described herein and the pharmaceutical formulation comprises a physiological pH, and administering at least one antibiotic or pharmaceutically acceptable salt thereof resulting in lower erythrocyte sedimentation rate (ESR) as compared to irrigation and debridement (I&D). In some embodiments, the combination treatment of administering both the peptide and the antibiotic results in reducing the ESR of the subject about 5 days from administration. In some embodiments, the combination treatment of administering both the peptide and the antibiotic results in reducing the ESR of a subject to normal levels after about 2 weeks from administration. In some embodiments, the combination treatment of administering both a pharmaceutical composition comprising a peptide described herein and an antibiotic result in reducing the ESR of the subject compared to administering pharmaceutical composition comprising the peptide or the antibiotic alone. In some embodiments, the combination treatment of administering both a pharmaceutical composition comprising a peptide described herein and an antibiotic result in reducing the ESR of the subject compared to administering pharmaceutical composition comprising the peptide or the antibiotic alone to a patient's base level or normal level. In some embodiments, the ESR of a patient is measured by Westergren method. In some embodiments, the ESR of a patient is measured by Wintrobe method.

In some embodiments, the combination treatment of administering both the peptide and the antibiotic results in a decrease of C-reactive protein levels after about 7 days from administration. In some embodiments, the method for treating a bacterial infection in a subject comprises administering the pharmaceutical composition to a subject resulting in lower CRP levels as compared to administering I&D. In some embodiments, the combination treatment of administering both a pharmaceutical composition comprising a peptide described herein and an antibiotic result in reducing the C-reactive protein (CRP) of the subject compared to administering pharmaceutical composition comprising the peptide or the antibiotic alone. In some embodiments, the combination treatment of administering both a pharmaceutical composition comprising a peptide described herein and an antibiotic results in reducing the C-reactive protein (CRP) of the subject compared to administering pharmaceutical composition comprising the peptide or the antibiotic alone to the patient's base level or normal level.

In some embodiments, the method disclosed herein may reduce the mass of the microbial biofilm. In some embodiments, the method disclosed herein may lead to at least about 0.5%, 1%, 2%, 3%, 4%, 5%, 6%, 7%, 8%, 9%, 10%, 12.5%, 15%, 17.5%, 20%, 22.5%, 25%, 27.5%, 30%, 32.5%, 35%, 37.5%, 40%, 42.5%, 45%, 47.5%, 50%, 60%, 70%, 80%, 90%, 91%, 92%, 93%, 94%, 95%, 96%, 97%, 98%, or 99% reduction in mass and any increments percentage therebetween.

In some embodiments, the method disclosed herein can lead to at least about 10% reduction in bacterial burden, at least about 20% reduction in bacterial burden, at least about 30% reduction in bacterial burden, at least about 40% reduction in bacterial burden, at least about 50% reduction in bacterial burden, at least about 60% reduction in bacterial burden, at least about 70% reduction in bacterial burden, at least about 80% reduction in bacterial burden, at least about 90% reduction in bacterial burden, at least about 90% reduction in bacterial burden, at least about 91% reduction in bacterial burden, at least about 92% reduction in bacterial burden, at least about 93% reduction in bacterial burden, at least about 94% reduction in bacterial burden, at least about 95% reduction in bacterial burden, at least about 96% reduction in bacterial burden, at least about 97% reduction in bacterial burden, at least about 98% reduction in bacterial burden, at least about 99% reduction in bacterial burden, at least about 99.9% reduction in bacterial burden, at least about 99.99% reduction in bacterial burden, or any increments therebetween.

In some embodiments, the method disclosed herein may further comprise an incision to open a site of infection. After an incision, a wash may be applied to the open site to treat or prevent infection. In some cases, a wash method may include irrigation of an open site with a wash. In some cases, a wash method may include drainage of an open site before, during, or after contacting the open site with a wash.

Administration of an antibiotic herein may be administered orally, rectally, or parenterally, in formulations containing conventionally acceptable carriers, adjuvants, and vehicles as desired. Administration may be intra-arterial, intravenous, intramuscular, oral, subcutaneous, intranasal, inhalational, any other administrative routes known to those skilled in the art, or any combination thereof. In some embodiments, administration may be injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous, inhalational, transdermal, transmucosal, sublingual, buccal and topical (including epicutaneous, dermal, enema, eye drops, ear drops, intranasal, vaginal), any other method of administration known to those skilled in the art, or any combinations thereof. In some exemplary embodiments, a route of administration may be via an intramuscular, an intravenous, a subcutaneous, or an intraperitoneal injection. In some exemplary embodiments, a route of administration may be intra-arterial, intravenous, intramuscular, oral, subcutaneous, or by inhalation. In some exemplary embodiments, a route of administration may be subcutaneous.

Administration of the pharmaceutical composition comprising a peptide or pharmaceutically acceptable salt thereof may be performed at least 1, 2, 3, 4, 5, 6, 7, 8, 9, 10, 11, 12, 13, 14, 15, 16, 17, 18, 19, 20, 21, 22, 23, 24, or 48 times a day. In some embodiments the pharmaceutical composition comprising a peptide or pharmaceutically acceptable salt thereof may be administered consecutively. In some embodiments the pharmaceutical composition comprising a peptide or pharmaceutically acceptable salt thereof may be administered non-consecutively. In some cases, pharmaceutical composition comprising a peptide or pharmaceutically acceptable salt may be delivered more than once a day. In some embodiments, peptide or pharmaceutically acceptable salt present may be delivered more than twice a day. In some cases, the pharmaceutical composition comprising a peptide or pharmaceutically acceptable salt may be administered over a course of at least about 1 day to at least about 12 months. In some cases, the pharmaceutical composition comprising a peptide or pharmaceutically acceptable salt may be administered over a course of at least about 2 months. In some cases, pharmaceutical composition comprising a peptide or pharmaceutically acceptable salt may be administered over a course of at least about 1 months. In some cases, pharmaceutical composition comprising a peptide or pharmaceutically acceptable salt may be administered over a course of at least about 2 weeks.

In some cases, administration of a pharmaceutical composition comprising a peptide may be performed at least 1, 2, 3, 4, 5, 6, 7, 8, 9, 10, 11, 12, 13, 14, 15, 16, 17, 18, 19, 20, or 21 times a week. In some cases, administration of a peptide, salt, or pharmaceutical composition comprising a peptide may be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, administration of the pharmaceutical composition comprising a peptide or pharmaceutically acceptable salt may occur over a time period of from at least about 0.5 min to at least about 30 min. In some embodiments, delivery of pharmaceutical composition comprising a peptide or pharmaceutically acceptable salt may occur over a time period of at least about 15 min.

In some cases, administration of the pharmaceutical composition comprising a peptide or pharmaceutically acceptable salt occurs over a time period of from at least about 0.5 min to at least about 1 min, from at least about 1 min to at least about 2 min, from at least about 2 min to at least about 3 min, from at least about 3 min to at least about 4 min, from at least about 4 min to at least about 5 min, from at least about 5 min to at least about 6 min, from at least about 6 min to at least about 7 min, from at least about 7 min to at least about 8 min, from at least about 8 min to at least about 9 min, from at least about 9 min to at least about 10 min, from at least about 10 min to at least about 11 min, from at least about 11 min to at least about 12 min, from at least about 12 min to at least about 13 min, from at least about 13 min to at least about 14 min, from at least about 14 min to at least about 15 min, from at least about 15 min to at least about 16 min, from at least about 16 min to at least about 17 min, from at least about 17 min to at least about 18 min, from at least about 18 min to at least about 19 min, from at least about 19 min to at least about 20 min, from at least about 21 min to at least about 22 min, from at least about 22 min to at least about 23 min, from at least about 23 min to at least about 24 min, from at least about 24 min to at least about 25 min, from at least about 25 min to at least about 26 min, from at least about 26 min to at least about 27 min, from at least about 27 min to at least about 28 min, from at least about 28 min to at least about 29 min, or from at least about 29 min to at least about 30 min. In some embodiments, administration of a pharmaceutical composition comprising a peptide or pharmaceutically acceptable salt occurs over a time period of at least 5 minutes, at least 7.5 minutes, at least 15 minutes, or at least 30 minutes. In some embodiments, administration of the pharmaceutical composition comprising a peptide or pharmaceutically acceptable salt occurs over a time period from about 0.1 minute to about 24 hours, about 0.1 minute to about 12 hours, about 0.1 minute to about 6 hours, about 0.1 minute to about 2 hours, about 0.1 minute to about 60 minutes, about 0.1 minute to about 30 minutes, about 0.1 minute to about 15 minutes, about 0.1 minute to about 10 minutes, about 0.1 minute to about 5 minutes, about 5 minute to about 15 minutes, about 5 minute to about 30 minutes, about 5 minute to about 60 minutes, about 5 minute to about 2 hours, about 5 minute to about 6 hours, about 5 minute to about 12 hours, or about 5 minute to 24 hours. In some embodiments, administration of a pharmaceutical composition comprising a peptide or pharmaceutically acceptable salt occurs over a time period from about 0.1 minute to about 60 minutes.

Administration of an antibiotic or pharmaceutically acceptable salt thereof may be performed at least 1, 2, 3, 4, 5, 6, 7, 8, 9, 10, 11, 12, 13, 14, 15, 16, 17, 18, 19, 20, 21, 22, 23, or 24 times a day. In some embodiments, an antibiotic or pharmaceutically acceptable salt thereof may be administered consecutively. In some embodiments, an antibiotic or pharmaceutically acceptable salt thereof may be administered non-consecutively. In some cases, administering an antibiotic or pharmaceutically acceptable salt may be more than once a day. In some embodiments, administering an antibiotic or pharmaceutically acceptable salt may be more than twice a day. In some cases, administration of an antibiotic or pharmaceutically acceptable salt may be over a course of at least about 1 day to at least about 12 months. In some cases, an antibiotic or pharmaceutically acceptable salt may be administered over a course of at least about 2 months. In some cases, an antibiotic or pharmaceutically acceptable salt may be administered over a course of at least about 1 months. In some cases, an antibiotic or pharmaceutically acceptable salt may be administered over a course of at least about 2 weeks. In some cases, administering an antibiotic or pharmaceutically acceptable salt may be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, delivery of pharmaceutical composition comprising at least one antibiotic or pharmaceutically acceptable salt thereof may occur over a time period of from at least about 0.5 min to at least about 600 min. In some embodiments, delivery of pharmaceutical composition comprising a peptide or pharmaceutically acceptable salt may occur over a time period of at least about 1 min, 1.5 min, 2 min, 5 min, 7.5 min, 15 min, 20 min, 30 min, 35 min, 40 min, 45 min, 50 min, 60 min, 2 hours, 3 hours, 4 hours, 5 hours, 10 hours, 20 hours, 30 hours, 40 hours, 50 hours, or 60 hours and any increments therebetween.

In some cases, delivery of pharmaceutical composition comprising at least one antibiotic or pharmaceutically acceptable salt thereof may occur over a time period of from at least about 0.5 min to at least about 1 min, from at least about 1 min to at least about 2 min, from at least about 2 min to at least about 3 min, from at least about 3 min to at least about 4 min, from at least about 4 min to at least about 5 min, from at least about 5 min to at least about 6 min, from at least about 6 min to at least about 7 min, from at least about 7 min to at least about 8 min, from at least about 8 min to at least about 9 min, from at least about 9 min to at least about 10 min, from at least about 10 min to at least about 11 min, from at least about 11 min to at least about 12 min, from at least about 12 min to at least about 13 min, from at least about 13 min to at least about 14 min, from at least about 14 min to at least about 15 min, from at least about 15 min to at least about 16 min, from at least about 16 min to at least about 17 min, from at least about 17 min to at least about 18 min, from at least about 18 min to at least about 19 min, from at least about 19 min to at least about 20 min, from at least about 21 min to at least about 22 min, from at least about 22 min to at least about 23 min, from at least about 23 min to at least about 24 min, from at least about 24 min to at least about 25 min, from at least about 25 min to at least about 26 min, from at least about 26 min to at least about 27 min, from at least about 27 min to at least about 28 min, from at least about 28 min to at least about 29 min, or from at least about 29 min to at least about 30 min.

In some embodiments, administration of an antibiotic or pharmaceutically acceptable salt thereof may be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some cases, a treatment duration may be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

In some embodiments of the method of administering a pharmaceutical composition, the peptide or pharmaceutically acceptable salt thereof as described herein is SA-5 (SEQ ID NO: 1) comprising physiological pH. In some embodiments of the method of administering a pharmaceutical composition, the peptide or pharmaceutically acceptable salt thereof as described herein is SA-5 (SEQ ID NO: 1). In some embodiments of the method of administering a pharmaceutical composition, the peptide or pharmaceutically acceptable salt thereof as described herein is LSA-5 (SEQ ID NO: 2). In some embodiments of the method of administering a pharmaceutical composition, the pharmaceutical composition comprising a peptide or pharmaceutically acceptable salt thereof as described herein is WLSA-5 (SEQ ID NO: 3). In some embodiments of the method of administering a pharmaceutical composition, the pharmaceutical composition comprising a peptide or pharmaceutically acceptable salt thereof as described herein is LBU-1 (SEQ ID NO: 4). In some embodiments of the method of administering a pharmaceutical composition, the pharmaceutical composition comprising a peptide or pharmaceutically acceptable salt thereof as described herein is LBU-2 (SEQ ID NO: 5). In some embodiments of the method of administering a pharmaceutical composition, the peptide or pharmaceutically acceptable salt thereof as described herein is LBU-3 (SEQ ID NO: 6). In some embodiments of the method of administering a pharmaceutical composition, the peptide or pharmaceutically acceptable salt thereof as described herein is LBU-3.5 (SEQ ID NO: 7). In some embodiments of the method of administering a pharmaceutical composition, the peptide or pharmaceutically acceptable salt thereof as described herein is LBU-4 (SEQ ID NO: 8). In some embodiments of the method of administering a pharmaceutical composition, the peptide or pharmaceutically acceptable salt thereof as described herein is WLBU-1 (SEQ ID NO: 9). In some embodiments of the method of administering a pharmaceutical composition, the peptide or pharmaceutically acceptable salt thereof as described herein is WLBU-2 (SEQ ID NO: 10). In some embodiments of the method of administering a pharmaceutical composition, the peptide or pharmaceutically acceptable salt thereof as described herein is WLBU-3 (SEQ ID NO: 11). In some embodiments of the method of administering a pharmaceutical composition, the peptide or pharmaceutically acceptable salt thereof as described herein is WLBU-4 (SEQ ID NO: 12). In some embodiments of the method of administering a pharmaceutical composition, the peptide or pharmaceutically acceptable salt thereof as described herein is WR-6 (SEQ ID NO: 13). In some embodiments of the method of administering a pharmaceutical composition, the peptide or pharmaceutically acceptable salt thereof as described herein is WR-12 (SEQ ID NO: 14). In some embodiments of the method of administering a pharmaceutical composition, the peptide or pharmaceutically acceptable salt thereof as described herein is WR-18 (SEQ ID NO: 15). In some embodiments of the method of administering a pharmaceutical composition, the peptide or pharmaceutically acceptable salt thereof as described herein is WR-24 (SEQ ID NO: 16). In some embodiments of the method of administering a pharmaceutical composition, the beta lactam as described herein is cefazolin.

Exemplary prosthetic joints are, but not limited to, replacement hip joint replacement, knee joint prosthetic joint, replacement shoulder joint, replacement elbow joint. In some embodiments, the prosthetic joint is a replacement hip joint replacement. In some embodiments, the prosthetic joint is a knee joint prosthetic joint. In some embodiments, the prosthetic joint is a replacement shoulder joint, replacement elbow joint.

### Dosage

In some embodiments, the pharmaceutical compositions described herein is in the form of a unit dose. In some embodiments, the method for treating or preventing a bacterial infection in a subject comprises administering a unit dose of a pharmaceutical composition comprising a peptide or pharmaceutically acceptable salt thereof and administering unit dose of at least one antibiotic.

In some cases, effective amounts of a peptide or pharmaceutically acceptable salt is at a concentration from at least about 0.01 μg/mL to at least about 100 mg/mL. In some embodiments, effective amounts of a peptide or pharmaceutically acceptable salt is at a concentration from at least about at least about 0.1 mg/mL to at least about 5 mg/mL. In some embodiments, effective amounts of a peptide or pharmaceutically acceptable salt is at a concentration from at least about at least about 0.5 mg/mL to at least about 1 mg/mL. In some embodiments, effective amounts of a peptide or pharmaceutically acceptable salt is at a concentration about 1 mg/mL.

In some cases, pharmaceutical composition comprising a peptide or pharmaceutically acceptable salt is present at a concentration from at least about 0.01 μg/mL to at least about 100 mg/mL. In some embodiments, the peptide or pharmaceutically acceptable salt is present at a concentration from at least about at least about 0.1 mg/mL to at least about 5 mg/mL. In some embodiments, the peptide or pharmaceutically acceptable salt is present at a concentration from at least about at least about 0.5 mg/mL to at least about 1 mg/mL. In some embodiments, the peptide or pharmaceutically acceptable salt is present at a concentration about 1 mg/mL. In some cases, pharmaceutical composition comprising a peptide or pharmaceutically acceptable salt is present at a concentration of about 3 mg/mL. In some cases, pharmaceutical composition comprising a peptide or pharmaceutically acceptable salt is present at a concentration of about 5 mg/mL. In some cases, pharmaceutical composition comprising a peptide or pharmaceutically acceptable salt is present at a concentration of about 10 mg/mL. In some cases, pharmaceutical composition comprising a peptide or pharmaceutically acceptable salt is present at a concentration of about 1 mg/mL, about 2 mg/mL, about 3 mg/mL, about 4 mg/mL, about 5 mg/mL, about 7.5 mg/mL, about 10 mg/mL, about 15 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, or any amounts therebetween.

In some cases, pharmaceutical composition comprising a peptide or pharmaceutically acceptable salt can exhibit antibacterial activity against a bacterial infection at a concentration from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

In some cases, administration of an antibiotic or pharmaceutically acceptable salt thereof is at a dose from about 0.001 mg/kg to at least about 100 mg/kg. In some embodiments, administration of an antibiotic or pharmaceutically acceptable salt thereof is at a dose of at least about 10 mg/kg.

In some cases, administration of an antibiotic or pharmaceutically acceptable salt thereof is at a dose from at least about 0.001 mg/kg to at least about 0.002 mg/kg, from at least about 0.002 mg/kg to at least about 0.003 mg/kg, from at least about 0.003 mg/kg to at least about 0.004 mg/kg, from at least about 0.004 mg/kg to at least about 0.005 mg/kg, from at least about 0.005 mg/kg to at least about 0.006 mg/kg, from at least about 0.006 mg/kg to at least about 0.007 mg/kg, from at least about 0.007 mg/kg to at least about 0.008 mg/kg, from at least about 0.008 mg/kg to at least about 0.009 mg/kg, from at least about 0.009 mg/kg to at least about 0.01 mg/kg, from at least about 0.01 mg/kg to at least about 0.02 mg/kg, from at least about 0.02 mg/kg to at least about 0.03 mg/kg, from at least about 0.03 mg/kg to at least about 0.04 mg/kg, from at least about 0.04 mg/kg to at least about 0.05 mg/kg, from at least about 0.05 mg/kg to at least about 0.06 mg/kg, from at least about 0.06 mg/kg to at least about 0.07 mg/kg, from at least about 0.07 mg/kg to at least about 0.08 mg/kg, from at least about 0.08 mg/kg to at least about 0.09 mg/kg, from at least about 0.09 mg/kg to at least about 0.1 mg/kg, from at least about 0.1 mg/kg to at least about 0.2 mg/kg, from at least about 0.2 mg/kg to at least about 0.3 mg/kg, from at least about 0.3 mg/kg to at least about 0.4 mg/kg, from at least about 0.4 mg/kg to at least about 0.5 mg/kg, from at least about 0.5 mg/kg to at least about 0.6 mg/kg, from at least about 0.6 mg/kg to at least about 0.7 mg/kg, from at least about 0.7 mg/kg to at least about 0.8 mg/kg, from at least about 0.8 mg/kg to at least about 0.9 mg/kg, from at least about 0.9 mg/kg to at least about 1 mg/kg, from at least about 1 mg/kg to at least about 2 mg/kg, from at least about 2 mg/kg to at least about 3 mg/kg, from at least about 3 mg/kg to at least about 4 mg/kg, from at least about 4 mg/kg to at least about 5 mg/kg, from at least about 5 mg/kg to at least about 6 mg/kg, from at least about 6 mg/kg to at least about 7 mg/kg, from at least about 7 mg/kg to at least about 8 mg/kg, from at least about 8 mg/kg to at least about 9 mg/kg, from at least about 9 mg/kg to at least about 10 mg/kg, from at least about 10 mg/kg to at least about 20 mg/kg from at least about 20 mg/kg to at least about 30 mg/kg, from at least about 30 mg/kg to at least about 40 mg/kg, from at least about 40 mg/kg to at least about 50 mg/kg, from at least about 50 mg/kg to at least about 60 mg/kg, from at least about 60 mg/kg to at least about 70 mg/kg, from at least about 70 mg/kg to at least about 80 mg/kg, from at least about 80 mg/kg to at least about 90 mg/kg, or from at least about 90 mg/kg to at least about 100 mg/kg.

In some cases, effective amounts of a peptide or pharmaceutically acceptable salt is at a concentration from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

In some embodiments, effective amounts of a peptide or pharmaceutically acceptable salt depend on the size of the treated object. In some embodiments, effective amounts of a peptide or pharmaceutically acceptable salt depend on the surface area of the treated object.

In some embodiments, effective amounts of a peptide or pharmaceutically acceptable salt may be from at least about 1 μL to at least about 2 μL, from at least about 2 μL to at least about 3 μL, from at least about 3 μL to at least about 4 μL, from at least about 4 μL to at least about 5 μL, from at least about 5 μL to at least about 6 μL, from at least about 6 μL to at least about 7 μL, from at least about 7 μL to at least about 8 μL, from at least about 8 μL to at least about 9 μL, from at least about 9 μL to at least about 10 μL, from at least about 10 μL to at least about 20 μL, from at least about 20 μL to at least about 30 μL, from at least about 30 μL to at least about 40 μL, from at least about 40 μL to at least about 50 μL, from at least about 50 μL to at least about 60 μL, from at least about 60 μL to at least about 70 μL, from at least about 70 μL to at least about 80 μL, from at least about 80 μL to at least about 90 μL, from at least about 90 μL to at least about 100 μL, from at least about 100 μL to at least about 200 μL, from at least about 200 μL to at least about 300 μL, from at least about 300 μL to at least about 400 μL, from at least about 400 μL to at least about 500 μL, from at least about 500 μL to at least about 600 μL, from at least about 600 μL to at least about 700 μL, from at least about 700 μL to at least about 800 μL, from at least about 800 μL to at least about 900 μL, from at least about 900 μL to at least about 1 mL, from at least about 1 mL to at least about 2 mL, from at least about 2 mL to at least about 3 mL, from at least about 3 mL to at least about 4 mL, from at least about 4 mL to at least about 5 mL, from at least about 5 mL to at least about 6 mL, from at least about 6 mL to at least about 7 mL, from at least about 7 mL to at least about 8 mL, from at least about 8 mL to at least about 9 mL, from at least about 9 mL to at least about 10 mL, from at least about 10 mL to at least about 20 mL, from at least about 20 mL to at least about 30 mL, from at least about 30 mL to at least about 40 mL, from at least about 40 mL to at least about 50 mL, from at least about 50 mL to at least about 60 mL, from at least about 60 mL to at least about 70 mL, from at least about 70 mL to at least about 80 mL, from at least about 80 mL to at least about 90 mL, from at least about 90 mL to at least about 100 mL, from at least about 100 mL to at least about 200 mL, from at least about 200 mL to at least about 300 mL, from at least about 300 mL to at least about 400 mL, from at least about 400 mL to at least about 500 mL, from at least about 500 mL to at least about 600 mL, from at least about 600 mL to at least about 700 mL, from at least about 700 mL to at least about 800 mL, from at least about 800 mL to at least about 900 mL, from at least about 900 mL to at least about 1 L, from at least about 1 L to at least about 2 L, from at least about 2 L to at least about 3 L, from at least about 3 L to at least about 4 L, from at least about 4 L to at least about 5 L, from at least about 5 L to at least about 6 L, from at least about 6 L to at least about 7 L, from at least about 7 L to at least about 8 L, from at least about 8 L to at least about 9 L, from at least about 9 L to at least about 10 L, from at least about 10 L to at least about 20 L, from at least about 20 L to at least about 30 L, from at least about 30 L to at least about 40 L, from at least about 40 L to at least about 50 L, from at least about 50 L to at least about 60 L, from at least about 60 L to at least about 70 L, from at least about 70 L to at least about 80 L, from at least about 80 L to at least about 90 L, from at least about 90 L to at least about 100 L, from at least about 100 L to at least about 200 L, from at least about 200 L to at least about 300 L, from at least about 300 L to at least about 400 L, from at least about 400 L to at least about 500 L, from at least about 500 L to at least about 600 L, from at least about 600 L to at least about 700 L, from at least about 700 L to at least about 800 L, from at least about 800 L to at least about 900 L, from at least about 900 L to at least about 1 kL, from at least about 1 kL to at least about 2 kL, from at least about 2 kL to at least about 3 kL, from at least about 3 kL to at least about 4 kL, from at least about 4 kL to at least about 5 kL, from at least about 5 kL to at least about 6 kL, from at least about 6 kL to at least about 7 kL, from at least about 7 kL to at least about 8 kL, from at least about 8 kL to at least about 9 kL, or from at least about 9 kL to at least about 10 kL.

In some cases, effective amounts of at least one antibiotic or pharmaceutically acceptable salt thereof can be delivered at a dose from at least about 0.001 mg/kg to at least about 100 mg/kg of the object. In some embodiments, pharmaceutical composition comprising at least one antibiotic or pharmaceutically acceptable salt thereof is delivered at a dose of at least about 10 mg/kg of the object.

In some cases, effective amounts of an antibiotic or pharmaceutically acceptable salt thereof is delivered at a dose from at least about 0.001 mg/kg to at least about 0.002 mg/kg, from at least about 0.002 mg/kg to at least about 0.003 mg/kg, from at least about 0.003 mg/kg to at least about 0.004 mg/kg, from at least about 0.004 mg/kg to at least about 0.005 mg/kg, from at least about 0.005 mg/kg to at least about 0.006 mg/kg, from at least about 0.006 mg/kg to at least about 0.007 mg/kg, from at least about 0.007 mg/kg to at least about 0.008 mg/kg, from at least about 0.008 mg/kg to at least about 0.009 mg/kg, from at least about 0.009 mg/kg to at least about 0.01 mg/kg, from at least about 0.01 mg/kg to at least about 0.02 mg/kg, from at least about 0.02 mg/kg to at least about 0.03 mg/kg, from at least about 0.03 mg/kg to at least about 0.04 mg/kg, from at least about 0.04 mg/kg to at least about 0.05 mg/kg, from at least about 0.05 mg/kg to at least about 0.06 mg/kg, from at least about 0.06 mg/kg to at least about 0.07 mg/kg, from at least about 0.07 mg/kg to at least about 0.08 mg/kg, from at least about 0.08 mg/kg to at least about 0.09 mg/kg, from at least about 0.09 mg/kg to at least about 0.1 mg/kg, from at least about 0.1 mg/kg to at least about 0.2 mg/kg, from at least about 0.2 mg/kg to at least about 0.3 mg/kg, from at least about 0.3 mg/kg to at least about 0.4 mg/kg, from at least about 0.4 mg/kg to at least about 0.5 mg/kg, from at least about 0.5 mg/kg to at least about 0.6 mg/kg, from at least about 0.6 mg/kg to at least about 0.7 mg/kg, from at least about 0.7 mg/kg to at least about 0.8 mg/kg, from at least about 0.8 mg/kg to at least about 0.9 mg/kg, from at least about 0.9 mg/kg to at least about 1 mg/kg, from at least about 1 mg/kg to at least about 2 mg/kg, from at least about 2 mg/kg to at least about 3 mg/kg, from at least about 3 mg/kg to at least about 4 mg/kg, from at least about 4 mg/kg to at least about 5 mg/kg, from at least about 5 mg/kg to at least about 6 mg/kg, from at least about 6 mg/kg to at least about 7 mg/kg, from at least about 7 mg/kg to at least about 8 mg/kg, from at least about 8 mg/kg to at least about 9 mg/kg, from at least about 9 mg/kg to at least about 10 mg/kg, from at least about 10 mg/kg to at least about 20 mg/kg from at least about 20 mg/kg to at least about 30 mg/kg, from at least about 30 mg/kg to at least about 40 mg/kg, from at least about 40 mg/kg to at least about 50 mg/kg, from at least about 50 mg/kg to at least about 60 mg/kg, from at least about 60 mg/kg to at least about 70 mg/kg, from at least about 70 mg/kg to at least about 80 mg/kg, from at least about 80 mg/kg to at least about 90 mg/kg, or from at least about 90 mg/kg to at least about 100 mg/kg.

In some cases, effective amounts of an antibiotic or pharmaceutically acceptable salt is at a concentration from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

In some embodiments, effective amounts of an antibiotic or pharmaceutically acceptable salt depend on the size of the treated object. In some embodiments, effective amounts of a peptide or pharmaceutically acceptable salt depend on the surface area of the treated object.

In some embodiments, effective amounts of an antibiotic or pharmaceutically acceptable salt thereof may be from at least about 1 μL to at least about 2 μL, from at least about 2 μL to at least about 3 μL, from at least about 3 μL to at least about 4 μL, from at least about 4 μL to at least about 5 μL, from at least about 5 μL to at least about 6 μL, from at least about 6 μL to at least about 7 μL, from at least about 7 μL to at least about 8 μL, from at least about 8 μL to at least about 9 μL, from at least about 9 μL to at least about 10 μL, from at least about 10 μL to at least about 20 μL, from at least about 20 μL to at least about 30 μL, from at least about 30 μL to at least about 40 μL, from at least about 40 μL to at least about 50 μL, from at least about 50 μL to at least about 60 μL, from at least about 60 μL to at least about 70 μL, from at least about 70 μL to at least about 80 μL, from at least about 80 μL to at least about 90 μL, from at least about 90 μL to at least about 100 μL, from at least about 100 μL to at least about 200 μL, from at least about 200 μL to at least about 300 μL, from at least about 300 μL to at least about 400 μL, from at least about 400 μL to at least about 500 μL, from at least about 500 μL to at least about 600 μL, from at least about 600 μL to at least about 700 μL, from at least about 700 μL to at least about 800 μL, from at least about 800 μL to at least about 900 μL, from at least about 900 μL to at least about 1 mL, from at least about 1 mL to at least about 2 mL, from at least about 2 mL to at least about 3 mL, from at least about 3 mL to at least about 4 mL, from at least about 4 mL to at least about 5 mL, from at least about 5 mL to at least about 6 mL, from at least about 6 mL to at least about 7 mL, from at least about 7 mL to at least about 8 mL, from at least about 8 mL to at least about 9 mL, from at least about 9 mL to at least about 10 mL, from at least about 10 mL to at least about 20 mL, from at least about 20 mL to at least about 30 mL, from at least about 30 mL to at least about 40 mL, from at least about 40 mL to at least about 50 mL, from at least about 50 mL to at least about 60 mL, from at least about 60 mL to at least about 70 mL, from at least about 70 mL to at least about 80 mL, from at least about 80 mL to at least about 90 mL, from at least about 90 mL to at least about 100 mL, from at least about 100 mL to at least about 200 mL, from at least about 200 mL to at least about 300 mL, from at least about 300 mL to at least about 400 mL, from at least about 400 mL to at least about 500 mL, from at least about 500 mL to at least about 600 mL, from at least about 600 mL to at least about 700 mL, from at least about 700 mL to at least about 800 mL, from at least about 800 mL to at least about 900 mL, from at least about 900 mL to at least about 1 L, from at least about 1 L to at least about 2 L, from at least about 2 L to at least about 3 L, from at least about 3 L to at least about 4 L, from at least about 4 L to at least about 5 L, from at least about 5 L to at least about 6 L, from at least about 6 L to at least about 7 L, from at least about 7 L to at least about 8 L, from at least about 8 L to at least about 9 L, from at least about 9 L to at least about 10 L, from at least about 10 L to at least about 20 L, from at least about 20 L to at least about 30 L, from at least about 30 L to at least about 40 L, from at least about 40 L to at least about 50 L, from at least about 50 L to at least about 60 L, from at least about 60 L to at least about 70 L, from at least about 70 L to at least about 80 L, from at least about 80 L to at least about 90 L, from at least about 90 L to at least about 100 L, from at least about 100 L to at least about 200 L, from at least about 200 L to at least about 300 L, from at least about 300 L to at least about 400 L, from at least about 400 L to at least about 500 L, from at least about 500 L to at least about 600 L, from at least about 600 L to at least about 700 L, from at least about 700 L to at least about 800 L, from at least about 800 L to at least about 900 L, from at least about 900 L to at least about 1 kL, from at least about 1 kL to at least about 2 kL, from at least about 2 kL to at least about 3 kL, from at least about 3 kL to at least about 4 kL, from at least about 4 kL to at least about 5 kL, from at least about 5 kL to at least about 6 kL, from at least about 6 kL to at least about 7 kL, from at least about 7 kL to at least about 8 kL, from at least about 8 kL to at least about 9 kL, or from at least about 9 kL to at least about 10 kL.

In some embodiments of a pharmaceutical composition comprising (i) a peptide or pharmaceutically acceptable salt thereof, and (ii) a beta lactam or pharmaceutically acceptable salt thereof, the concentration of the peptide or pharmaceutically acceptable salt thereof is from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

In some embodiments of a pharmaceutical composition comprising (i) a peptide or pharmaceutically acceptable salt thereof, and (ii) a beta lactam or pharmaceutically acceptable salt thereof, the dosage of the beta lactam or pharmaceutically acceptable salt thereof is from at least about 0.001 mg/kg to at least about 0.002 mg/kg, from at least about 0.002 mg/kg to at least about 0.003 mg/kg, from at least about 0.003 mg/kg to at least about 0.004 mg/kg, from at least about 0.004 mg/kg to at least about 0.005 mg/kg, from at least about 0.005 mg/kg to at least about 0.006 mg/kg, from at least about 0.006 mg/kg to at least about 0.007 mg/kg, from at least about 0.007 mg/kg to at least about 0.008 mg/kg, from at least about 0.008 mg/kg to at least about 0.009 mg/kg, from at least about 0.009 mg/kg to at least about 0.01 mg/kg, from at least about 0.01 mg/kg to at least about 0.02 mg/kg, from at least about 0.02 mg/kg to at least about 0.03 mg/kg, from at least about 0.03 mg/kg to at least about 0.04 mg/kg, from at least about 0.04 mg/kg to at least about 0.05 mg/kg, from at least about 0.05 mg/kg to at least about 0.06 mg/kg, from at least about 0.06 mg/kg to at least about 0.07 mg/kg, from at least about 0.07 mg/kg to at least about 0.08 mg/kg, from at least about 0.08 mg/kg to at least about 0.09 mg/kg, from at least about 0.09 mg/kg to at least about 0.1 mg/kg, from at least about 0.1 mg/kg to at least about 0.2 mg/kg, from at least about 0.2 mg/kg to at least about 0.3 mg/kg, from at least about 0.3 mg/kg to at least about 0.4 mg/kg, from at least about 0.4 mg/kg to at least about 0.5 mg/kg, from at least about 0.5 mg/kg to at least about 0.6 mg/kg, from at least about 0.6 mg/kg to at least about 0.7 mg/kg, from at least about 0.7 mg/kg to at least about 0.8 mg/kg, from at least about 0.8 mg/kg to at least about 0.9 mg/kg, from at least about 0.9 mg/kg to at least about 1 mg/kg, from at least about 1 mg/kg to at least about 2 mg/kg, from at least about 2 mg/kg to at least about 3 mg/kg, from at least about 3 mg/kg to at least about 4 mg/kg, from at least about 4 mg/kg to at least about 5 mg/kg, from at least about 5 mg/kg to at least about 6 mg/kg, from at least about 6 mg/kg to at least about 7 mg/kg, from at least about 7 mg/kg to at least about 8 mg/kg, from at least about 8 mg/kg to at least about 9 mg/kg, from at least about 9 mg/kg to at least about 10 mg/kg, from at least about 10 mg/kg to at least about 20 mg/kg from at least about 20 mg/kg to at least about 30 mg/kg, from at least about 30 mg/kg to at least about 40 mg/kg, from at least about 40 mg/kg to at least about 50 mg/kg, from at least about 50 mg/kg to at least about 60 mg/kg, from at least about 60 mg/kg to at least about 70 mg/kg, from at least about 70 mg/kg to at least about 80 mg/kg, from at least about 80 mg/kg to at least about 90 mg/kg, or from at least about 90 mg/kg to at least about 100 mg/kg.

In some embodiments of a pharmaceutical composition comprising (i) a peptide or pharmaceutically acceptable salt thereof, and (ii) a beta lactam or pharmaceutically acceptable salt thereof, effective amounts of a peptide or pharmaceutically acceptable salt is at a concentration from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

In some embodiments of a pharmaceutical composition comprising (i) a peptide or pharmaceutically acceptable salt thereof, and (ii) a beta lactam or pharmaceutically acceptable salt thereof, effective amounts of the pharmaceutical composition may be from at least about 1 μL to at least about 2 μL, from at least about 2 μL to at least about 3 μL, from at least about 3 μL to at least about 4 μL, from at least about 4 μL to at least about 5 μL, from at least about 5 μL to at least about 6 μL, from at least about 6 μL to at least about 7 μL, from at least about 7 μL to at least about 8 μL, from at least about 8 μL to at least about 9 μL, from at least about 9 μL to at least about 10 μL, from at least about 10 μL to at least about 20 μL, from at least about 20 μL to at least about 30 μL, from at least about 30 μL to at least about 40 μL, from at least about 40 μL to at least about 50 μL, from at least about 50 μL to at least about 60 μL, from at least about 60 μL to at least about 70 μL, from at least about 70 μL to at least about 80 μL, from at least about 80 μL to at least about 90 μL, from at least about 90 μL to at least about 100 μL, from at least about 100 μL to at least about 200 μL, from at least about 200 μL to at least about 300 μL, from at least about 300 μL to at least about 400 μL, from at least about 400 μL to at least about 500 μL, from at least about 500 μL to at least about 600 μL, from at least about 600 μL to at least about 700 μL, from at least about 700 μL to at least about 800 μL, from at least about 800 μL to at least about 900 μL, from at least about 900 p L to at least about 1 mL, from at least about 1 mL to at least about 2 mL, from at least about 2 mL to at least about 3 mL, from at least about 3 mL to at least about 4 mL, from at least about 4 mL to at least about 5 mL, from at least about 5 mL to at least about 6 mL, from at least about 6 mL to at least about 7 mL, from at least about 7 mL to at least about 8 mL, from at least about 8 mL to at least about 9 mL, from at least about 9 mL to at least about 10 mL, from at least about 10 mL to at least about 20 mL, from at least about 20 mL to at least about 30 mL, from at least about 30 mL to at least about 40 mL, from at least about 40 mL to at least about 50 mL, from at least about 50 mL to at least about 60 mL, from at least about 60 mL to at least about 70 mL, from at least about 70 mL to at least about 80 mL, from at least about 80 mL to at least about 90 mL, from at least about 90 mL to at least about 100 mL, from at least about 100 mL to at least about 200 mL, from at least about 200 mL to at least about 300 mL, from at least about 300 mL to at least about 400 mL, from at least about 400 mL to at least about 500 mL, from at least about 500 mL to at least about 600 mL, from at least about 600 mL to at least about 700 mL, from at least about 700 mL to at least about 800 mL, from at least about 800 mL to at least about 900 mL, from at least about 900 mL to at least about 1 L, from at least about 1 L to at least about 2 L, from at least about 2 L to at least about 3 L, from at least about 3 L to at least about 4 L, from at least about 4 L to at least about 5 L, from at least about 5 L to at least about 6 L, from at least about 6 L to at least about 7 L, from at least about 7 L to at least about 8 L, from at least about 8 L to at least about 9 L, from at least about 9 L to at least about 10 L, from at least about 10 L to at least about 20 L, from at least about 20 L to at least about 30 L, from at least about 30 L to at least about 40 L, from at least about 40 L to at least about 50 L, from at least about 50 L to at least about 60 L, from at least about 60 L to at least about 70 L, from at least about 70 L to at least about 80 L, from at least about 80 L to at least about 90 L, from at least about 90 L to at least about 100 L, from at least about 100 L to at least about 200 L, from at least about 200 L to at least about 300 L, from at least about 300 L to at least about 400 L, from at least about 400 L to at least about 500 L, from at least about 500 L to at least about 600 L, from at least about 600 L to at least about 700 L, from at least about 700 L to at least about 800 L, from at least about 800 L to at least about 900 L, from at least about 900 L to at least about 1 kL, from at least about 1 kL to at least about 2 kL, from at least about 2 kL to at least about 3 kL, from at least about 3 kL to at least about 4 kL, from at least about 4 kL to at least about 5 kL, from at least about 5 kL to at least about 6 kL, from at least about 6 kL to at least about 7 kL, from at least about 7 kL to at least about 8 kL, from at least about 8 kL to at least about 9 kL, or from at least about 9 kL to at least about 10 kL.

In some embodiments of a pharmaceutical composition comprising (i) a peptide or pharmaceutically acceptable salt thereof, and (ii) an antibiotic or pharmaceutically acceptable salt thereof, effective amounts of an antibiotic or pharmaceutically acceptable salt thereof is delivered at a dose from at least about 0.001 mg/kg to at least about 0.002 mg/kg, from at least about 0.002 mg/kg to at least about 0.003 mg/kg, from at least about 0.003 mg/kg to at least about 0.004 mg/kg, from at least about 0.004 mg/kg to at least about 0.005 mg/kg, from at least about 0.005 mg/kg to at least about 0.006 mg/kg, from at least about 0.006 mg/kg to at least about 0.007 mg/kg, from at least about 0.007 mg/kg to at least about 0.008 mg/kg, from at least about 0.008 mg/kg to at least about 0.009 mg/kg, from at least about 0.009 mg/kg to at least about 0.01 mg/kg, from at least about 0.01 mg/kg to at least about 0.02 mg/kg, from at least about 0.02 mg/kg to at least about 0.03 mg/kg, from at least about 0.03 mg/kg to at least about 0.04 mg/kg, from at least about 0.04 mg/kg to at least about 0.05 mg/kg, from at least about 0.05 mg/kg to at least about 0.06 mg/kg, from at least about 0.06 mg/kg to at least about 0.07 mg/kg, from at least about 0.07 mg/kg to at least about 0.08 mg/kg, from at least about 0.08 mg/kg to at least about 0.09 mg/kg, from at least about 0.09 mg/kg to at least about 0.1 mg/kg, from at least about 0.1 mg/kg to at least about 0.2 mg/kg, from at least about 0.2 mg/kg to at least about 0.3 mg/kg, from at least about 0.3 mg/kg to at least about 0.4 mg/kg, from at least about 0.4 mg/kg to at least about 0.5 mg/kg, from at least about 0.5 mg/kg to at least about 0.6 mg/kg, from at least about 0.6 mg/kg to at least about 0.7 mg/kg, from at least about 0.7 mg/kg to at least about 0.8 mg/kg, from at least about 0.8 mg/kg to at least about 0.9 mg/kg, from at least about 0.9 mg/kg to at least about 1 mg/kg, from at least about 1 mg/kg to at least about 2 mg/kg, from at least about 2 mg/kg to at least about 3 mg/kg, from at least about 3 mg/kg to at least about 4 mg/kg, from at least about 4 mg/kg to at least about 5 mg/kg, from at least about 5 mg/kg to at least about 6 mg/kg, from at least about 6 mg/kg to at least about 7 mg/kg, from at least about 7 mg/kg to at least about 8 mg/kg, from at least about 8 mg/kg to at least about 9 mg/kg, from at least about 9 mg/kg to at least about 10 mg/kg, from at least about 10 mg/kg to at least about 20 mg/kg from at least about 20 mg/kg to at least about 30 mg/kg, from at least about 30 mg/kg to at least about 40 mg/kg, from at least about 40 mg/kg to at least about 50 mg/kg, from at least about 50 mg/kg to at least about 60 mg/kg, from at least about 60 mg/kg to at least about 70 mg/kg, from at least about 70 mg/kg to at least about 80 mg/kg, from at least about 80 mg/kg to at least about 90 mg/kg, or from at least about 90 mg/kg to at least about 100 mg/kg.

In some embodiments of a pharmaceutical composition comprising (i) a peptide or pharmaceutically acceptable salt thereof, and (ii) an antibiotic or pharmaceutically acceptable salt thereof, effective amounts of an antibiotic or pharmaceutically acceptable salt is at a concentration from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

### Bacterial Infection

Provided herein are pharmaceutical compositions and methods for treating or preventing bacterial infections in subjects. In some embodiments, the bacterial infection comprise an infection from a bacteria of Staphylococcus aureus, methicillin resistant Staphylococcus aureus, Streptococcus pneumonia, carbapenem-resistant Enteroacteriaceae, Staphylococcus epidermidis, Staphylococcus salivarius, Corynebacterium minutissium, Corynebacterium pseudodiphtheriae, Corynebacterium stratium, Corynebacterium group G1, Corynebacterium group G2, Streptococcus pneumonia, Streptococcus mitis, Streptococcus sanguis, Escherichia coli, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces spp., Porphyromonas spp., Prevotella melaninogenicus, Helicobacter pylori, Helicobacter felis, or Campylobacter jejuni, or any other bacteria known to those skilled in the art to be capable of infecting a patient or subject and combinations thereof. In some embodiments, the bacterial infection comprise an infection from a bacteria of Staphylococcus aureus, Staphylococcus epidermidis, Staphylococcus lugdenensis, Staphylococcus haemolyticus, Staphylococcus hominis, Staphylococcus saprophyticus, Staphylococcus simulans, Staphylococcus warnerii, Staphylococcus capitis, Staphylococcus caprae, Staphylococcus pettenkoferi, Streptococcus pyogenes, Streptococcus agalactiae, Streptococcus pneumoniae, Group C streptococci, Streptococcus constellatus, Enterococcus faecalis, Enterococcus faecium, Corynebacterium jeikeium, Lactobacillus acidophilus, Listeria monocytogenes, Escherichia coli, Klebsiella pneumoniae, Klebsiella oxytoca, Acinetobacter baumannii, Acinetobacter nosocomialis, Acinetobacter pittii, Acinetobacter haemolyticus, Acinetobacter radioresistens, Acinetobacter ursingii, Pseudomonas aeruginosa, Enterobacter cloacae, Enterobacter aerogenes, Stenotrophomonas maltophilia, Citrobacter freundii, Citrobacter koseri, Citrobacter sedlakii, Citrobacter braakii, Morganella morganii, Providencia rettgeri, Providencia stuartii, Salmonella typhimurium, Shigella dysenteriae, Moraxella catarrhalis, Neisseria gonorrhoeae, Propionibacterium acnes, Clostridioides difficile, Clostridioides perfringens, Bacteroides fragilis, Prevotella bivia, Eggerthella lenta, Peptostreptococcus anaerobius, Haemophilus parainfluenzae, Staphylococcus haemolyticus, Streptococcus dysgalactiae, and any combination thereof. In some embodiments, a bacteria species can be resistant to at least one antibiotic. In some embodiment, a bacteria can be resistant to at least one antibiotic comprising Ampicillin, Bactrum, Clindamycin, Colistin, Erythromycin, Gentamycin, Imipenem, Levofloxacin, Linezolid, Oxacillin, Rifampin, Sulbactam, Trimethoprim, Tigecycline, Tetracycline, Vancomycin, or a combination thereof. In some embodiment, the bacterial infection comprise an infection from a bacteria of Enterococcus species, Staphylococcus aureus, Klebsiella pneumoniae, Acinetobacter species, Pseudomonas species, Enterococcus species, (ESKAPE) and any combination thereof. In some embodiments, the bacterial infection is a Staphylococcus aureus infection. In some embodiments, an amount of bacteria of the bacterial infection is defined as bacterial burden. In some embodiments, a bacterial burden can be an implant bacterial burden, bone bacterial burden, or both. In some embodiments, a bacterial burden can be an implant bacterial burden. In some embodiments, a bacterial burden can be a bone bacterial burden. In some embodiments, a bacterial burden is measured as colony-forming unit per milliliter (CFU/mL) by colony-forming unit analysis.

In some embodiments, a bacterial infection can lead to a biofilm. In some embodiments, the biofilm is a mature biofilm.

In some embodiments, a bacterial infection can arise from a wound, surgical procedure, an implanted medical device, a biological transplant, or any other cause of infection known to those skilled in the art. In some embodiments, the bacterial infection from an implanted device occurs at the location of the implanted device. In some embodiments, an implanted device can lead to periprosthetic joint infection. In some embodiments, the bacterial infection is a periprosthetic joint infection. In some embodiments, a periprosthetic joint infection can lead to a biofilm. In some embodiments, the periprosthetic joint infection is caused by antibiotic-tolerant bacteria.

### Kits

Disclosed herein are kits. A kit can comprise a pharmaceutical composition comprising a peptide or pharmaceutically acceptable salt thereof and at least one antibiotic or pharmaceutically acceptable salt as described herein. A kit can comprise a pharmaceutical composition comprising a peptide or pharmaceutically acceptable salt thereof and at least one β lactam antibiotic or pharmaceutically acceptable salt as described herein. In some embodiments, a kit can further comprise an aqueous carrier. In some embodiments, a kit can further comprise a second aqueous carrier. In some embodiments, a kit can further comprise a container for mixing a pharmaceutical composition comprising a peptide or pharmaceutically acceptable salt thereof and an aqueous carrier. In some respects, a pharmaceutical composition can be packaged in a container. In some respects, a kit can further comprise instructions that direct administration of a unit dose of a peptide or formulation to a subject. In some respects, a kit can comprise a peptide disclosed herein and instructions for the use thereof.

Methods of making a kit can include placing a pharmaceutical composition comprising a peptide or pharmaceutically acceptable salt thereof and at least one antibiotic or pharmaceutically acceptable salt thereof as described herein in a container for packaging. A method can further comprise an inclusion of instructions for use. In some cases, instructions for use can direct administration of a unit dose of a pharmaceutical composition to a subject.

### Terminology

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases.

As used herein, the terms “biofilm”, “microbial film”, “microbial biofilm”, “bacterial film”, refers to any film comprising microorganisms and their excretions.

As used herein, the terms “comprising,” “comprise” or “comprised,” and variations thereof, in reference to elements of an item, composition, apparatus, method, process, system, claim etc. are intended to be open-ended, meaning that the item, composition, apparatus, method, process, system, claim etc. includes those elements and other elements can be included and still fall within the scope/definition of the described item, composition, apparatus, method, process, system, claim etc. As used herein, “a” or “an” means one or more. As used herein “another” may mean at least a second or more.

As used herein, the term “object” refers to any object with a surface. Some embodiments in the present disclosure may be applied to the surface of an object to prevent or to treat microbial biofilm. In some embodiments, the object can a solid object, a liquid object, a hard object, a soft object, a metallic object, a polymeric object, a ceramic object, a composite object, a biological object, members of the animal kingdom, a human being, a biological transplant object, a replaced joint, or any other object with a surface on which some of the disclosed methods and the formulations can be applied.

As used herein, the terms “patient” or “subject” generally refer to any individual that has, may have, or may be suspected of having a disease condition (e.g., a bacterial infection). In some cases, the bacterial infection may be caused by surgeries, physical wounds, etc. The subject may be an animal. The animal can be a mammal, such as a human, non-human primate, a rodent such as a mouse or rat, a dog, a cat, pig, sheep, or rabbit. Animals can be fish, reptiles, or others. Animals can be neonatal, infant, adolescent, or adult animals. The subject may be a living organism. The subject may be a human. Humans can be greater than or equal to 1, 2, 5, 10, 20, 30, 40, 50, 60, 65, 70, 75, 80 or more years of age. A human may be from about 18 to about 90 years of age. A human may be from about 18 to about 30 years of age. A human may be from about 30 to about 50 years of age. A human may be from about 50 to about 90 years of age. The subject may have one or more risk factors of a condition and be asymptomatic. The subject may be asymptomatic of a condition. The subject may have one or more risk factors for a condition. The subject may be symptomatic for a condition. The subject may be symptomatic for a condition and have one or more risk factors of the condition. The subject may have or be suspected of having a disease, such as an infection. The subject may be a patient being treated for a disease, such as an infection. The subject may be predisposed to a risk of developing a disease such as a bacterial infection. The subject may be in remission from a disease, such as a bacterial infection. The subject may not have a bacterial infection. The subject may be healthy.

As used herein, a “pharmaceutically acceptable excipient”, “aqueous carrier” or “pharmaceutically acceptable aqueous carrier” refer to solvents or dispersion media, and the like, that are physiologically compatible and known to those skilled in the art. Examples of pharmaceutically acceptable carriers include one or more of water, saline, phosphate buffered saline, dextrose, glycerol, ethanol, and the like, as well as combinations thereof. Pharmaceutically acceptable carriers may further comprise minor amounts of auxiliary substances such as wetting or emulsifying agents, preservatives or buffers, which enhance the shelf life or effectiveness of the active agent.

As used herein, an “effective amount” of an active agent can refer to an amount that is effective to achieve a desired result, e.g., in reducing microbial biofilm mass. An effective amount comprises a therapeutically effective amount, which refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology). An effective amount of a given active agent can vary with respect to factors such as the type and severity of the disorder or disease being treated and the age, gender, and weight of the patient.

The term “homology” can refer to a % identity of a polypeptide to a reference polypeptide. As a practical matter, whether any particular polypeptide can be at least 50%, 60%, 70%, 80%, 85%, 90%, 92%, 95%, 96%, 97%, 98% or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters can be set such that the percentage of identity is calculated over the full length of the reference amino acid sequence and that gaps in homology of up to 5% of the total number of amino acid residues in the reference sequence are allowed.

For example, in a specific embodiment the identity between a reference sequence (query sequence, i.e., a sequence of the present invention) and a subject sequence, also referred to as a global sequence alignment, may be determined using the FASTDB computer program based on the algorithm of Brutlag et al. (Comp. App. Biosci. 6:237-245 (1990)). In some embodiments, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some embodiments, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 amino acid residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for preventing a formation of a microbial biofilm or reducing said microbial biofilm mass on an object, comprising administering effective amounts of a peptide or pharmaceutically acceptable salt thereof and a beta-lactam antibiotic or pharmaceutically acceptable salt thereof, wherein the peptide and beta-lactam antibiotic are applied to the object for a sufficient amount of time such that said microbial biofilm is not formed or said mass of said microbial biofilm is reduced.

Embodiment 2. The method of Embodiment 1, wherein said object is a biological transplant.

Embodiment 3. The method of Embodiment 3, wherein said biological transplant is a replaced joint.

Embodiment 4. The method of Embodiment 1, wherein said microbial biofilm is not formed.

Embodiment 5. The method of Embodiment 1, wherein said mass of said microbial biofilm is reduced by at least about 10%.

Embodiment 6. The method of Embodiment 1, wherein said microbial biofilm is not formed or said mass of said microbial biofilm is reduced by at least about 20%.

Embodiment 7. The method of Embodiment 1, wherein said microbial biofilm is not formed or said mass of said microbial biofilm is reduced by at least about 30%.

Embodiment 8. The method of Embodiment 1, wherein said microbial biofilm is not formed or said mass of said microbial biofilm is reduced by at least about 50%.

Embodiment 9. The method of Embodiment 1, wherein said peptide or pharmaceutically acceptable salt thereof is applied simultaneously with said beta-lactam antibiotic or pharmaceutically acceptable salt thereof.

Embodiment 10. The method of Embodiment 1, wherein said peptide or pharmaceutically acceptable salt thereof is applied prior to said beta-lactam antibiotic or pharmaceutically acceptable salt thereof.

Embodiment 11. The method of Embodiment 1, wherein said peptide or pharmaceutically acceptable salt thereof is applied subsequent to said beta-lactam antibiotic or pharmaceutically acceptable salt thereof.

Embodiment 12. The method of Embodiment 10, wherein said peptide or pharmaceutically acceptable salt thereof is applied at least about 12 hours prior to said beta-lactam antibiotic or pharmaceutically acceptable salt thereof.

Embodiment 13. The method of Embodiment 10, wherein said peptide or pharmaceutically acceptable salt thereof is applied at most about 30 minutes prior to said beta-lactam antibiotic or pharmaceutically acceptable salt thereof.

Embodiment 14. The method of Embodiment 11, wherein said peptide or pharmaceutically acceptable salt thereof is applied at least about 12 hours subsequent to said beta-lactam antibiotic or pharmaceutically acceptable salt thereof.

Embodiment 15. The method of Embodiment 11, wherein said peptide or pharmaceutically acceptable salt thereof is applied at most about 30 minutes subsequent to said beta-lactam antibiotic or pharmaceutically acceptable salt thereof.

Embodiment 16. A method for treating or preventing an infection in a subject, comprising:
administering an effective amount of a pharmaceutical composition to said subject, wherein said pharmaceutical composition comprises a peptide or pharmaceutically acceptable salt thereof; and
administering an effective amount of a beta-lactam antibiotic or pharmaceutically acceptable salt thereof, thereby treating or preventing said infection in said subject.

Embodiment 17. The method of Embodiment 16, wherein said beta-lactam antibiotic or pharmaceutically acceptable salt thereof is applied simultaneously with said pharmaceutical composition.

Embodiment 18. The method of Embodiment 16, wherein said beta-lactam antibiotic or pharmaceutically acceptable salt thereof is applied prior to said pharmaceutical composition.

Embodiment 19. The method of Embodiment 16, wherein said beta-lactam antibiotic or pharmaceutically acceptable salt thereof is applied subsequent to said pharmaceutical composition.

Embodiment 20. The method of either of Embodiments 16-18, wherein said pharmaceutical composition comprises a pH value of at least about 7.2 to at least about 7.9.

Embodiment 21. The method of either of Embodiments 16-18, wherein said pharmaceutical composition comprises a pH value of at least about 7.3 to at least about 7.5.

Embodiment 22. The method of either of Embodiments 16-18, wherein said pharmaceutical composition comprises a pH value of at least about 7.4.

Embodiment 23. The method of Embodiment 16, further comprising reducing a bacterial burden of said infection in said subject.

Embodiment 24. The method of Embodiment 16, further comprising reducing erythrocyte sedimentation rate (ESR) in said subject.

Embodiment 25. The method of Embodiment 24, wherein said ESR is measured by Westergren method.

Embodiment 26. The method of Embodiment 24, wherein said ESR is measured by Wintrobe method.

Embodiment 27. The method of Embodiment 16, further comprising reducing C-reactive protein expression levels in said subject.

Embodiment 28. The method of Embodiment 16, further comprising increasing a survival rate of said subject.

Embodiment 29. The method of Embodiment 16, wherein administering comprises injection.

Embodiment 30. The method of Embodiment 29, wherein said injection comprises an intra-articular injection.

Embodiment 31. The method of Embodiment 16, further comprising incising and draining said infection in said subject prior to step a), thereby resulting in an open wound.

Embodiment 32. The method of Embodiment 31, in a), further comprising debriding said open wound.

Embodiment 33. The method of Embodiment 31, in a), further comprising washing said open wound.

Embodiment 34. The method of Embodiment 16, wherein said beta-lactam antibiotic or pharmaceutically acceptable salt thereof is administered intra-arterially, intravenously, intramuscularly, orally, subcutaneously, rectally, as inhalatory administration, or any combination thereof.

Embodiment 35. The method of Embodiment 34, wherein said beta-lactam antibiotic or pharmaceutically acceptable salt thereof is administered subcutaneously.

Embodiment 36. The method of Embodiment 16, in a), wherein said pharmaceutical composition is administered to said subject more than once per day.

Embodiment 37. The method of Embodiment 16, in a), wherein said pharmaceutical composition is administered to said subject more than twice per day.

Embodiment 38. The method of Embodiment 16, in b), wherein said beta-lactam antibiotic or pharmaceutically acceptable salt thereof is administered to said subject more than once per day.

Embodiment 39. The method of Embodiment 16, in b), wherein said beta-lactam antibiotic or pharmaceutically acceptable salt thereof is administered to said subject more than twice per day.

Embodiment 40. The method of Embodiment 16, wherein said infection is at a location of an implanted device.

Embodiment 41. The method of Embodiment 16, wherein said infection is a periprosthetic joint infection.

Embodiment 42. The method of Embodiment 16, wherein said infection forms a microbial biofilm.

Embodiment 43. The method of any of Embodiments 16-42, wherein said infection is caused by antibiotic-tolerant bacteria.

Embodiment 44. The method of Embodiment 40, further comprising incising and draining said location of said implanted device prior to a).

Embodiment 45. The method of Embodiment 40, in a), further comprising washing said implanted device.

Embodiment 46. The method of Embodiment 40, further comprising irrigating and debriding said location of said implanted device prior to step a).

Embodiment 47. The method of Embodiment 40, in a), further comprising washing said implanted device.

Embodiment 48. The method of any one of Embodiments 1 to 47, wherein said peptide or pharmaceutically acceptable salt thereof has at least 70% sequence identity to a polypeptide sequence of: SA-5 (SEQ ID NO: 1); LSA-5 (SEQ ID NO: 2); WLSA-5 (SEQ ID NO: 3); LBU-1 (SEQ ID NO: 4); LBU-2 (SEQ ID NO: 5); LBU-3 (SEQ ID NO: 6); LBU-3.5 (SEQ ID NO: 7); LBU-4 (SEQ ID NO: 8); WLBU-1 (SEQ ID NO: 9); WLBU-2 (SEQ ID NO: 10); WLBU-3 (SEQ ID NO: 11); WLBU-4 (SEQ ID NO: 12); WR6 (SEQ ID NO: 13); WR12 (SEQ ID NO: 14); WR18 (SEQ ID NO: 15); or WR 24 (SEQ ID NO: 16).

Embodiment 49. The method of Embodiment 48, wherein said peptide or pharmaceutically acceptable salt is WLBU-2 (SEQ ID NO: 10).

Embodiment 50. The method of Embodiment 48, wherein said peptide or pharmaceutically acceptable is WR12 (SEQ ID NO: 14).

Embodiment 51. The method of any one of Embodiments 1 to 50, wherein said beta-lactam antibiotic is selected from the group consisting of Cephalexin, Cephradine, Cephalexin, Cefadroxil, Cephalexin, Cefazolin, BETA-lactam antibiotic C, Cephalothin, Cefapirin, Cefuroxime, Cefprozil, Loracarbef, Cefuroxime, Cefoxitin, Cefotetan, Cefaclor, Cefamandole, Ceftriaxone, Cefdinir, Cefixime, Cefpodoxime, Cefditoren, Ceftibuten, Ceftazidime, Cefotaxime, Cefoperazone, Ceftizoxime, Cefepime, Cefiderocol, Cefpirome, Ceftaroline, Benzathine, Benzylpenicillin, Phenoxymethylpenicillin, Procaine penicillin, Pheneticillin, Cloxacillin, Dicloxacillin, Flucloxacillin, Methicillin, Nafcillin, Oxacillin, Temocillin, Amoxillin, Ampicillin, Mecillinam, Piperacillin, Carbenicillin, Ticarillin, Carbenicillin, Ticarcillin, Azlocillin, Mezlocillin, Piperacillin, Biapenem, Doripenem, Ertapenem, Faropenem, Imipenem, Meropenem, Panipenem, Razupenem, Tebipenem, Thienamycin, Aztreonam, Tigermonam, Nocardicin A, Tabtoxinine beta-lactam, Clavulanic acid, Tazobactam, Sulbactam, Avibactam, and any combination thereof.

Embodiment 52. The method of Embodiment 51, wherein said beta-lactam antibiotic is Cefazolin.

Embodiment 53. The method of any one of Embodiments 1 to 52, wherein said pharmaceutical composition is in a form of a liquid.

Embodiment 54. The method of any one of Embodiments 1 to 53, wherein said pharmaceutical composition further comprising a pH buffer.

Embodiment 55. The method of Embodiment 54, wherein said pH buffer is bicarbonate, phosphate, lactate, acetate, tris(hydroxymethyl)aminomethane, or any combination thereof.

Embodiment 56. The method of Embodiment 55, wherein said phosphate pH buffer is Dulbecco's phosphate buffered saline (dPBS).

Embodiment 57. The method of Embodiment 1 to 56, wherein said pharmaceutical composition further comprises a pH adjusting agent.

Embodiment 58. The method of Embodiment 57, wherein said pH adjusting agent is sodium hydroxide.

Embodiment 59. The method of Embodiment 1 to 58, wherein said peptide or pharmaceutically acceptable salt thereof is administered at a concentration from at least about 0.01 μg/mL to at least about 100 mg/mL.

Embodiment 60. The method of Embodiment 59, wherein said peptide or pharmaceutically acceptable salt thereof is administered at a concentration from at least about 0.1 mg/mL to at least about 5 mg/mL.

Embodiment 61. The method of Embodiment 59, wherein said peptide or pharmaceutically acceptable salt thereof is administered at a concentration from at least about 0.5 mg/mL to at least about 1 mg/mL.

Embodiment 62. The method of Embodiment 59, wherein said peptide or pharmaceutically acceptable salt thereof is present at a concentration at least about 1 mg/mL.

Embodiment 63. The method of any one of Embodiments 8 to 62, in a), wherein said pharmaceutical composition is administered for a sufficient of time for treating or preventing said infection.

Embodiment 64. The method of any of Embodiments 1-53, wherein said sufficient time is at least about 0.5 min to at least about 600 min.

Embodiment 65. The method of any of Embodiments 1-53, wherein said sufficient time is at least about 5 min to at least about 15 min.

Embodiment 66. The method of any of Embodiments 1-53, wherein said sufficient time is at least about 15 min.

Embodiment 67. The method of any one of Embodiments 1 to 66, wherein said beta-lactam antibiotic or pharmaceutically acceptable salt thereof is present as a dose of least about 0.001 mg/kg to at least about 100 mg/kg.

Embodiment 68. The method of any of Embodiments 1-67, wherein said beta-lactam antibiotic or pharmaceutically acceptable salt thereof is present at a dose of least about 10 mg/mL.

Embodiment 69. The method of any one of Embodiments 1-68, wherein said method lasts over a course of at least about 1 day to at least about 10 years.

Embodiment 70. The method of Embodiment 69, wherein said course is at least about 12 months.

Embodiment 71. The method of Embodiment 69, wherein said course is at least about 2 months.

Embodiment 72. The method of Embodiment 69, wherein said course is at least about 1 month.

Embodiment 73. The method of Embodiment 69, wherein said course is at least about 2 weeks.

Embodiment 74. A pharmaceutical composition comprising: a peptide or pharmaceutically acceptable salt thereof, wherein said peptide or pharmaceutically acceptable salt thereof has at least 70% sequence identity to a polypeptide sequence of SA-5 (SEQ ID NO: 1); LSA-5 (SEQ ID NO: 2); WLSA-5 (SEQ ID NO: 3); LBU-1 (SEQ ID NO: 4); LBU-2 (SEQ ID NO: 5); LBU-3 (SEQ ID NO: 6); LBU-3.5 (SEQ ID NO: 7); LBU-4 (SEQ ID NO: 8); WLBU-1 (SEQ ID NO: 9); WLBU-2 (SEQ ID NO: 10); WLBU-3 (SEQ ID NO: 11); WLBU-4 (SEQ ID NO: 12); WR6 (SEQ ID NO: 13); WR12 (SEQ ID NO: 14); WR18 (SEQ ID NO: 15); or WR 24 (SEQ ID NO: 16); and a beta-lactam antibiotic or pharmaceutically acceptable salt thereof; wherein said pharmaceutical composition comprises a pH value of at least about 7.2 to 7.9.

Embodiment 75. The pharmaceutical composition of Embodiment 74, wherein said peptide or pharmaceutically acceptable salt is WLBU-2 (SEQ ID NO: 10).

Embodiment 76. The pharmaceutical composition of Embodiment 74, wherein said peptide or pharmaceutically acceptable salt is WR12 (SEQ ID NO: 14).

Embodiment 77. The pharmaceutical composition of any one of Embodiments 74 to 76, wherein said beta-lactam antibiotic or pharmaceutically acceptable salt thereof comprises Cephalexin, Cephradine, Cephalexin, Cefadroxil, Cephalexin, Cefazolin, BETA-lactam antibiotic C, Cephalothin, Cefapirin, Cefuroxime, Cefprozil, Loracarbef, Cefuroxime, Cefoxitin, Cefotetan, Cefaclor, Cefamandole, Ceftriaxone, Cefdinir, Cefixime, Cefpodoxime, Cefditoren, Ceftibuten, Ceftazidime, Cefotaxime, Cefoperazone, Ceftizoxime, Cefepime, Cefiderocol, Cefpirome, Ceftaroline, Benzathine, Benzylpenicillin, Phenoxymethylpenicillin, Procaine penicillin, Pheneticillin, Cloxacillin, Dicloxacillin, Flucloxacillin, Methicillin, Nafcillin, Oxacillin, Temocillin, Amoxillin, Ampicillin, Mecillinam, Piperacillin, Carbenicillin, Ticarillin, Carbenicillin, Ticarcillin, Azlocillin, Mezlocillin, Piperacillin, Biapenem, Doripenem, Ertapenem, Faropenem, Imipenem, Meropenem, Panipenem, Razupenem, Tebipenem, Thienamycin, Aztreonam, Tigermonam, Nocardicin A, Tabtoxinine beta-lactam, Clavulanic acid, Tazobactam, Sulbactam, Avibactam, or any combination thereof.

Embodiment 78. The pharmaceutical composition 77, wherein said beta-lactam antibiotic or pharmaceutically acceptable salt thereof is Cefazolin.

Embodiment 79. The pharmaceutical composition of any one of Embodiments 74 to 78, wherein said pharmaceutical composition is in a form of a liquid.

Embodiment 80. The pharmaceutical composition of any one of Embodiments 74 to 79, further comprising a pH buffer.

Embodiment 81. The pharmaceutical composition of Embodiment 80, wherein said pH buffer is bicarbonate, phosphate, lactate, acetate, tris(hydroxymethyl)aminomethane, or any combination thereof.

Embodiment 82. The pharmaceutical composition of Embodiment 81, wherein said phosphate pH buffer is Dulbecco's phosphate buffered saline (dPBS).

Embodiment 83. The pharmaceutical composition of Embodiment 74, wherein said pH value is at least about 7.4.

Embodiment 84. The pharmaceutical composition of any one of Embodiments 74 to 83, further comprising a pH adjusting agent.

Embodiment 85. The pharmaceutical composition of Embodiment 84, wherein said pH adjusting agent is sodium hydroxide.

Embodiment 86. The pharmaceutical composition of any one of Embodiments 74 to 85, wherein said peptide or pharmaceutically acceptable salt thereof has at a concentration from at least about 0.01 μg/mL to at least about 100 mg/mL.

Embodiment 87. The pharmaceutical composition of Embodiment 86, wherein said peptide or pharmaceutically acceptable salt thereof has a concentration from at least about 0.1 mg/mL to at least about 5 mg/mL.

Embodiment 88. The pharmaceutical composition of Embodiment 86, wherein said peptide or pharmaceutically acceptable salt thereof has a concentration from at least about 0.5 mg/mL to at least about 1 mg/mL.

Embodiment 89. The pharmaceutical composition of Embodiment 86, wherein said peptide or pharmaceutically acceptable salt thereof has a concentration at least about 1 mg/mL.

Embodiment 90. The pharmaceutical composition of Embodiments 74 to 89, wherein said beta-lactam antibiotic or pharmaceutically acceptable salt thereof is present at a dose of least about 0.001 mg/kg to at least about 100 mg/kg.

Embodiment 91. The pharmaceutical composition 90, wherein said beta-lactam antibiotic or pharmaceutically acceptable salt thereof is present at a dose of least about 10 mg/mL.

Embodiment 92. The pharmaceutical composition of any one of Embodiments 74 to 91, wherein said pharmaceutical composition is in form of a unit dose.

Embodiment 93. A kit, comprising said pharmaceutical composition or said unit dose of any one of Embodiments 74 to 92 and instructions for use of said pharmaceutical composition for treatment or preventing an infection.

Embodiment 94. A method for treating or preventing periprosthetic joint infection in a patient in need thereof, wherein a prosthetic joint is implanted in said patient; the method comprising administering: (i) a pharmaceutical composition comprising: (a) a peptide or pharmaceutically acceptable salt thereof, wherein said peptide has at least 70% sequence identity to a polypeptide sequence of: SA-5 (SEQ ID NO: 1); LSA-5 (SEQ ID NO: 2); WLSA-5 (SEQ ID NO: 3); LBU-1 (SEQ ID NO: 4); LBU-2 (SEQ ID NO: 5); LBU-3 (SEQ ID NO: 6); LBU-3.5 (SEQ ID NO: 7); LBU-4 (SEQ ID NO: 8); WLBU-1 (SEQ ID NO: 9); WLBU-2 (SEQ ID NO: 10); WLBU-3 (SEQ ID NO: 11); WLBU-4 (SEQ ID NO: 12); WR6 (SEQ ID NO: 13); WR12 (SEQ ID NO: 14); WR18 (SEQ ID NO: 15); or WR 24 (SEQ ID NO: 16); and (b) an aqueous carrier; wherein said pharmaceutical composition is in a form of a liquid, wherein said pharmaceutical composition is locally administered to said prosthetic joint in vivo; and (ii) an antibiotic or pharmaceutically acceptable salt thereof; and wherein administration of said pharmaceutical composition and said antibiotic reduces a bacterial burden of said periprosthetic joint infection to a greater extent as compared to administering (i) or (ii) alone.

Embodiment 95. The method of Embodiment 94, wherein said bacterial burden comprises implant bacterial burden, bone bacterial burden, or both.

Embodiment 96. The method of Embodiment 94 or 95, wherein said reduction of said bacterial burden is measured as colony-forming unit per milliliter (CFU/mL) by colony-forming unit (CFU) analysis.

Embodiment 97. The method of Embodiment 96, wherein said bacterial burden comprises implant bacterial burden, and wherein said method reduces said CFU/mL by at least 2.5 log.

Embodiment 98. The method of any one of Embodiments 94 to 97, wherein said pharmaceutical composition is administered prior to said antibiotic administration.

Embodiment 99. The method of any one of Embodiments 94 to 97, wherein said pharmaceutical composition is administered simultaneously with said antibiotic administration.

Embodiment 100. The method of any one of Embodiments 94 to 97, wherein said pharmaceutical composition is administered subsequent to said antibiotic administration.

Embodiment 101. The method of any one of Embodiments 94 to 100, wherein said periprosthetic joint infection further comprises a biofilm.

Embodiment 102. The method of Embodiment 101, wherein said method reduces said biofilm by at least about 10%, at least about 20%, at least about 30%, or at least about 50%.

Embodiment 103. The method of Embodiment 101 or 102, wherein said biofilm is a mature biofilm.

Embodiment 104. The method of any one of Embodiments 101 to 103, wherein the method partially disrupts or destroys said biofilm.

Embodiment 105. The method of any one Embodiments 94 to 104, wherein said locally administering comprises irrigating said prosthetic joint with said pharmaceutical composition, wherein said prosthetic joint is exposed.

Embodiment 106. The method of any one Embodiments 94 to 105, wherein locally administering the pharmaceutical composition to the prosthetic joint in vivo occurs for at least 5 minutes, at least 7.5 minutes, at least 15 minutes, or at least 30 minutes.

Embodiment 107. The method of any one Embodiments 94 to 106, wherein locally administering the pharmaceutical composition to the prosthetic joint in vivo occurs for from about 0.1 minute to about 24 hours or about 0.1 minute to about 60 minutes.

Embodiment 108. The method of any one Embodiments 94 to 106, wherein said antibiotic or pharmaceutically acceptable salt thereof is administered intra-arterially, intravenously, intramuscularly, orally, subcutaneously, rectally, as inhalatory administration, or any combination thereof.

Embodiment 109. The method of any one Embodiments 94 to 108, wherein said antibiotic or pharmaceutically acceptable salt thereof is administered subcutaneously.

Embodiment 110. The method of any one Embodiments 94 to 109, wherein said peptide or pharmaceutically acceptable salt thereof is applied at least about 12 hours prior to said antibiotic or pharmaceutically acceptable salt thereof.

Embodiment 111. The method of any one Embodiments 94 to 109, wherein said peptide or pharmaceutically acceptable salt thereof is applied at most about 30 minutes prior to said antibiotic or pharmaceutically acceptable salt thereof.

Embodiment 112. The method of any one Embodiments 94 to 109, wherein said peptide or pharmaceutically acceptable salt thereof is applied at least about 12 hours subsequent to said antibiotic or pharmaceutically acceptable salt thereof.

Embodiment 113. The method of any one Embodiments 94 to 109, wherein said peptide or pharmaceutically acceptable salt thereof is applied at most about 30 minutes subsequent to said antibiotic or pharmaceutically acceptable salt thereof.

Embodiment 114. The method of any one Embodiments 94 to 113, wherein said peptide or pharmaceutically acceptable salt is WLBU-2 (SEQ ID NO: 10).

Embodiment 115. The method of any one Embodiments 94 to 113, wherein said peptide or pharmaceutically acceptable is WR12 (SEQ ID NO: 14).

Embodiment 116. The method of any one Embodiments 94 to 115, wherein said aqueous carrier comprises phosphate buffered saline (PBS), Dulbecco's PBS, normal saline, water, lactated Ringer's solution, or aqueous sodium bicarbonate.

Embodiment 117. The method of any one Embodiments 94 to 116, wherein the aqueous carrier comprises Dulbecco's PBS, normal saline, water, or aqueous sodium bicarbonate.

Embodiment 118. The method of any one Embodiments 94 to 117, wherein said pharmaceutical composition comprises a pH value of at least about 5 to at least about 10.

Embodiment 119. The method of Embodiment 118, wherein said pharmaceutical composition comprises a pH value of at least about 7.2 to at least about 8.5.

Embodiment 120. The method of any one Embodiments 94 to 119, wherein said peptide or pharmaceutically acceptable salt thereof is present in said pharmaceutical composition at a concentration from at least about 0.01 μg/mL to at least about 100 mg/mL.

Embodiment 121. The method of any one Embodiments 94 to 120, wherein said peptide or pharmaceutically acceptable salt thereof is present in said pharmaceutical composition at a concentration from at least about 1 mg/mL to at least about 10 mg/mL.

Embodiment 122. The method of any one Embodiments 94 to 121, wherein said peptide or pharmaceutically acceptable salt thereof is present in said pharmaceutical composition at a concentration at about 1 mg/mL, about 3 mg/mL, or about 10 mg/mL.

Embodiment 123. The method of any one Embodiments 94 to 122, wherein said antibiotic is a beta-lactam antibiotic.

Embodiment 124. The method of Embodiment 123, wherein said beta-lactam antibiotic is selected from the group consisting of: Amoxillin, Ampicillin, Avibactam, Azlocillin, Aztreonam, Benzathine, Benzylpenicillin, Beta-lactam antibiotic C, Biapenem, Carbenicillin, Cefaclor, Cefadroxil, Cefamandole, Cefapirin, Cefazolin, Cefdinir, Cefditoren, Cefepime, Cefiderocol, Cefixime, Cefoperazone, Cefotetan, Cefotaxime, Cefoxitin, Cefpirome, Cefpodoxime, Cefprozil, Ceftriaxone, Ceftaroline, Ceftazidime, Ceftibuten, Ceftizoxime, Cefuroxime, Cephalexin, Cephalothin, Cephradine, Clavulanic acid, Cloxacillin, Dicloxacillin, Doripenem, Ertapenem, Faropenem, Flucloxacillin, Imipenem, Loracarbef, Mecillinam, Meropenem, Methicillin, Mezlocillin, Nafcillin, Nocardicin A, Oxacillin, Panipenem, Pheneticillin, Phenoxymethylpenicillin, Piperacillin, Procaine penicillin, Razupenem, Sulbactam, Tabtoxinine beta-lactam, Tazobactam, Tebipenem, Temocillin, Ticarillin, Thienamycin, Ticarcillin, Tigermonam, any salts thereof, and any combination thereof.

Embodiment 125. The method of Embodiment 123 or 124, wherein said beta-lactam antibiotic is Cefazolin.

Embodiment 126. The method of any one Embodiments 94 to 122, wherein said antibiotic is selected from the group consisting of: Amikacin, Ampicillin, Avibactim, Azithromycin, Aztreonam, Cefepime, Cefpodoxime, Ceftazidime, Ceftriaxone, Ciprofloxacin, Colistin, Daptomycin, Doxycycline, Eravacycline, Gentamicin, Levofloxacin, Linezolid, Meropenem, Penicillin G, Piperacillin, Plazomicin, Sulbactam, Tazobactam, Tetracycline, Tobramycin, Vaborbactam, Vancomycin, any salts thereof, and any combination thereof.

Embodiment 127. The method of any one Embodiments 94 to 126, wherein said antibiotic or pharmaceutically acceptable salt thereof is administered at a concentration of at least about 0.01 μg/mL to at least about 100 mg/mL.

Embodiment 128. The method of any one Embodiments 94 to 127, wherein said antibiotic or pharmaceutically acceptable salt thereof is administered at a concentration of at least about 1 mg/mL to at least about 10 mg/mL.

Embodiment 129. The method of any one Embodiments 94 to 128, wherein said antibiotic or pharmaceutically acceptable salt thereof is administered at a concentration of at least about 0.01 μg/kg to at least about 100 mg/kg.

Embodiment 130. The method of any one Embodiments 94 to 129, wherein said antibiotic or pharmaceutically acceptable salt thereof is administered at a concentration of at least about 1 mg/mL to at least about 10 mg/mL.

Embodiment 131. The method of any one Embodiments 94 to 130, wherein said bacterial burden comprises a bacterial species is selected from the group consisting of Staphylococcus aureus, Staphylococcus epidermidis, Staphylococcus lugdenensis, Staphylococcus haemolyticus, Staphylococcus hominis, Staphylococcus saprophyticus, Staphylococcus simulans, Staphylococcus warnerii, Staphylococcus capitis, Staphylococcus caprae, Staphylococcus pettenkoferi, Streptococcus pyogenes, Streptococcus agalactiae, Streptococcus pneumoniae, Group C streptococci, Streptococcus constellatus, Enterococcus faecalis, Enterococcus faecium, Corynebacterium jeikeium, Lactobacillus acidophilus, Listeria monocytogenes, Escherichia coli, Klebsiella pneumoniae, Klebsiella oxytoca, Acinetobacter baumannii, Acinetobacter nosocomialis, Acinetobacter pittii, Acinetobacter haemolyticus, Acinetobacter radioresistens, Acinetobacter ursingii, Pseudomonas aeruginosa, Enterobacter cloacae, Enterobacter aerogenes, Stenotrophomonas maltophilia, Citrobacter freundii, Citrobacter koseri, Citrobacter sedlakii, Citrobacter braakii, Morganella morganii, Providencia rettgeri, Providencia stuartii, Salmonella typhimurium, Shigella dysenteriae, Moraxella catarrhalis, Neisseria gonorrhoeae, Propionibacterium acnes, Clostridioides difficile, Clostridioides perfringens, Bacteroides fragilis, Prevotella bivia, Eggerthella lenta, Peptostreptococcus anaerobius, Haemophilus parainfluenzae, Staphylococcus haemolyticus, Streptococcus dysgalactiae, and any combination thereof.

Embodiment 132. The method of Embodiment 131, wherein said bacterial species is resistant to at least one antibiotic.

Embodiment 133. The method of Embodiment 132, wherein said at least one antibiotic comprises Ampicillin, Bactrum, Clindamycin, Colistin, Erythromycin, Gentamycin, Imipenem, Levofloxacin, Linezolid, Oxacillin, Rifampin, Sulbactam, Trimethoprim, Tigecycline, Tetracycline, Vancomycin, or a combination thereof.

Embodiment 134. The method of any one Embodiments 94 to 133, wherein said bacterial burden comprises a bacterial selected from the group consisting of Enterococcus species, Staphylococcus aureus, Klebsiella pneumoniae, Acinetobacter species, Pseudomonas species, Enterococcus species, and any combination thereof.

Embodiment 135. The method of any one Embodiments 94 to 134, further comprising debriding said prosthetic joint prior to administration of said pharmaceutical composition.

Embodiment 136. The method of any one Embodiments 94 to 135, wherein said prosthetic joint comprises replacement knee joint, replace hip joint, or replacement shoulder joint.

Embodiment 137. The method of any one Embodiments 94-136, further comprising reducing erythrocyte sedimentation rate (ESR) in said subject to a greater extent as compared to administering (i) or (ii) alone.

Embodiment 138. The method of Embodiment 137, wherein said ESR is measured by Westergren method.

Embodiment 139. The method of Embodiment 137, wherein said ESR is measured by Wintrobe method.

Embodiment 140. The method of any one Embodiments 94-139, further comprising reducing C-reactive protein expression levels in said subject a greater extent as compared to administering (i) or (ii) alone.

Embodiment 141. The method of any one Embodiments 94-140, further comprising increasing a survival rate of said subject.

Embodiment 142. The method of any one Embodiments 94-141, wherein said pharmaceutical composition is administered to said subject more than once per day.

Embodiment 143. The method of any one Embodiments 94-142, wherein said antibiotic or pharmaceutically acceptable salt thereof is administered to said subject more than once per day.

Embodiment 144. The method of any one Embodiments 94-143, wherein said antibiotic or pharmaceutically acceptable salt thereof is administered to said subject more than twice per day.

Embodiment 145. A pharmaceutical composition comprising: a peptide or pharmaceutically acceptable salt thereof, wherein said peptide or pharmaceutically acceptable salt thereof has at least 70% sequence identity to a polypeptide sequence of SA-5 (SEQ ID NO: 1); LSA-5 (SEQ ID NO: 2); WLSA-5 (SEQ ID NO: 3); LBU-1 (SEQ ID NO: 4); LBU-2 (SEQ ID NO: 5); LBU-3 (SEQ ID NO: 6); LBU-3.5 (SEQ ID NO: 7); LBU-4 (SEQ ID NO: 8); WLBU-1 (SEQ ID NO: 9); WLBU-2 (SEQ ID NO: 10); WLBU-3 (SEQ ID NO: 11); WLBU-4 (SEQ ID NO: 12); WR6 (SEQ ID NO: 13); WR12 (SEQ ID NO: 14); WR18 (SEQ ID NO: 15); or WR 24 (SEQ ID NO: 16); an aqueous carrier; and an antibiotic or pharmaceutically acceptable salt thereof; wherein said pharmaceutical composition is in a form of a liquid.

Embodiment 146. The pharmaceutical composition of Embodiment 145, wherein said peptide or pharmaceutically acceptable salt is WLBU-2 (SEQ ID NO: 10).

Embodiment 147. The pharmaceutical composition of Embodiment 145, wherein said peptide or pharmaceutically acceptable salt is WR12 (SEQ ID NO: 14).

Embodiment 148. The pharmaceutical composition of any one of Embodiments 145-147, wherein said aqueous carrier comprises phosphate buffered saline (PBS), Dulbecco's PBS, normal saline, water, lactated Ringer's solution, or aqueous sodium bicarbonate.

Embodiment 149. The pharmaceutical composition of any one of Embodiments 145-148, wherein the aqueous carrier comprises Dulbecco's PBS, normal saline, water, or aqueous sodium bicarbonate.

Embodiment 150. The pharmaceutical composition of any one of Embodiments 145-149, wherein said pharmaceutical composition comprises a pH value of at least about 5 to at least about 10.

Embodiment 151. The pharmaceutical composition of any one of Embodiments 145-150, wherein said pharmaceutical composition comprises a pH value of at least about 7.2 to at least about 8.5.

Embodiment 152. The pharmaceutical composition of any one of Embodiments 145-151, wherein said peptide or pharmaceutically acceptable salt thereof is present in said pharmaceutical composition at a concentration from at least about 0.01 μg/mL to at least about 100 mg/mL.

Embodiment 153. The pharmaceutical composition of any one of Embodiments 145-152, wherein said peptide or pharmaceutically acceptable salt thereof is present in said pharmaceutical composition at a concentration from at least about 1 mg/mL to at least about 10 mg/mL.

Embodiment 154. The pharmaceutical composition of any one of Embodiments 145-153, wherein said peptide or pharmaceutically acceptable salt thereof is present in said pharmaceutical composition at a concentration at about 1 mg/mL, about 3 mg/mL, or about 10 mg/mL.

Embodiment 155. The pharmaceutical composition of any one of Embodiments 145-154, wherein said antibiotic is a beta-lactam antibiotic.

Embodiment 156. The pharmaceutical composition of Embodiment 155, wherein said beta-lactam antibiotic is selected from the group consisting of Amoxillin, Ampicillin, Avibactam, Azlocillin, Aztreonam, Benzathine, Benzylpenicillin, Beta-lactam antibiotic C, Biapenem, Carbenicillin, Cefaclor, Cefadroxil, Cefamandole, Cefapirin, Cefazolin, Cefdinir, Cefditoren, Cefepime, Cefiderocol, Cefixime, Cefoperazone, Cefotetan, Cefotaxime, Cefoxitin, Cefpirome, Cefpodoxime, Cefprozil, Ceftriaxone, Ceftaroline, Ceftazidime, Ceftibuten, Ceftizoxime, Cefuroxime, Cephalexin, Cephalothin, Cephradine, Clavulanic acid, Cloxacillin, Dicloxacillin, Doripenem, Ertapenem, Faropenem, Flucloxacillin, Imipenem, Loracarbef, Mecillinam, Meropenem, Methicillin, Mezlocillin, Nafcillin, Nocardicin A, Oxacillin, Panipenem, Pheneticillin, Phenoxymethylpenicillin, Piperacillin, Procaine penicillin, Razupenem, Sulbactam, Tabtoxinine beta-lactam, Tazobactam, Tebipenem, Temocillin, Ticarillin, Thienamycin, Ticarcillin, Tigermonam, any salts thereof, and any combination thereof.

Embodiment 157. The pharmaceutical composition of Embodiment 155 or 156, wherein said beta-lactam antibiotic is Cefazolin.

Embodiment 158. The pharmaceutical composition of any one of Embodiments 145 to 154, wherein said antibiotic is selected from the group consisting of: Amikacin, Ampicillin, Avibactim, Azithromycin, Aztreonam, Cefepime, Cefpodoxime, Ceftazidime, Ceftriaxone, Ciprofloxacin, Colistin, Daptomycin, Doxycycline, Eravacycline, Gentamicin, Levofloxacin, Linezolid, Meropenem, Penicillin G, Piperacillin, Plazomicin, Sulbactam, Tazobactam, Tetracycline, Tobramycin, Vaborbactam, Vancomycin, any salts thereof, and any combination thereof.

Embodiment 159. The pharmaceutical composition of any one of Embodiments 145 to 158, wherein said antibiotic or pharmaceutically acceptable salt thereof is present in said pharmaceutical composition at a concentration from at least about 0.01 μg/mL to at least about 100 mg/mL.

Embodiment 160. The pharmaceutical composition of any one of Embodiments 145 to 159, wherein said antibiotic or pharmaceutically acceptable salt thereof is present in said pharmaceutical composition at a concentration from at least about 1 mg/mL to at least about 10 mg/mL.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

In the following Examples 1-5, the administered WLBU-2 formulations were prepared as described in this paragraph. Exemplary pharmaceutical formulations comprising WLBU-2 were prepared at concentrations of 0.5 mg/mL and 1.0 mg/mL in vehicle (dPBS), by formulating under aseptic conditions. WLBU-2 was added while mixing to approximately 90% of volume with vehicle, and mixed until the solution was clear. The solution was adjusted to pH 7.4 using a 1% NaOH solution. dPBS was added quantity sufficient to final volume. The final pH of the solution was read and recorded.

### Example 1: Ex Vivo Treatment of Infection with Rabbit PJI Model

This example illustrates the antibacterial activity of an exemplary formulation of WLBU-2 in reducing colony-forming unit (CFU) bacterial burden of an implant ex vivo and bacterial burden bone ex vivo.

TABLE A
Experimental Conditions

| Group | Test | Dose Volume | Dose | Time of | |
|---|---|---|---|---|---|
| No. | material | (mL)/site | (mg/ml) | exposure | n |
| 1 | Untreated | 0 | 0 | 0 | 4 |
| | Ex vivo | | | | |
| 2 | WLBU-2 | 2 | 1 | 15 minutes | 5 |
| | (long) Ex | | | | |
| | vivo | | | | |

A bone tunnel in the tibial canal was created using a drill with a 1.2 mm or 1.6 mm tungsten carbide drill bit. The bone tunnel was then dried and treated to simulate acute human PJI following primary arthroplasty. A Kirschner wire implant was placed in the bone tunnel and the wound closed. Prior to closure of the superficial skin layer, 0.1 mL of 2×10⁶ planktonic bacteria (CFU/rabbit) in saline was injected into the joint space. A closure was performed, and a biofilm was allowed to become established over a period of 2 days.

In Group 1, the joint space was reopened and treated with irrigation and debridement (I&D) at 2 days post infection, then the implant was removed. In Group 2, the joint space was reopened and treated with I&D at 2 days post infection, the implant was removed, then the implant was treated with WLBU-2 solution in a tube at a concentration of 1 mg/ml concentration for 15 minutes.

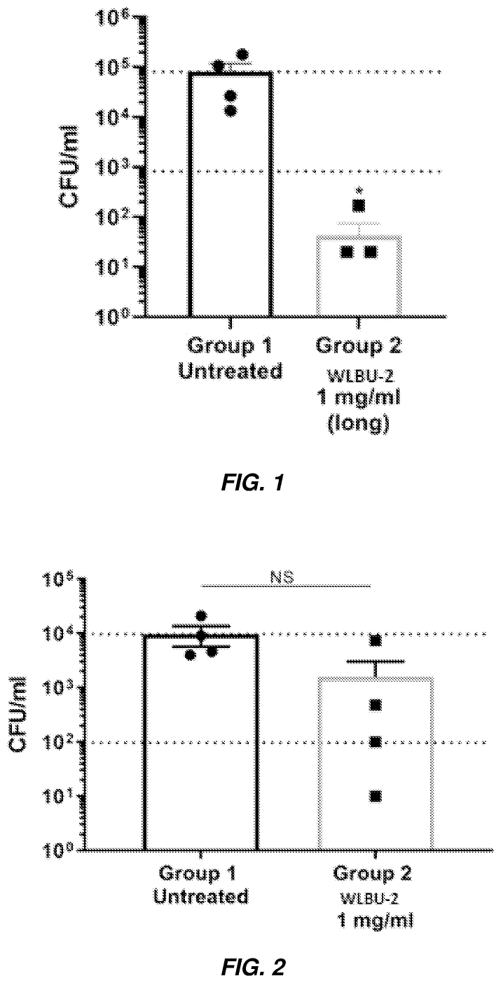

CFU analyses were performed with the infected implants from each group (FIG. 1). The WLBU-2 treated group showed greater than 2 log reduction in bacterial burden.

CFU analyses were also performed using bone samples from test subjects as seen in FIG. 2. There was no significant difference in bacterial burden between Group 1 and Group 2.

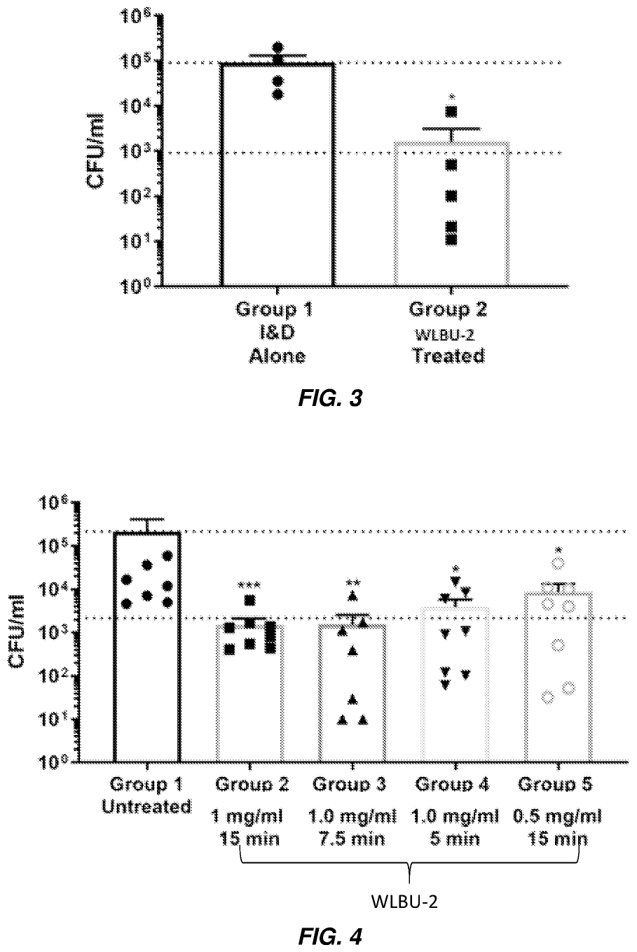

FIG. 3 shows the total CFU which includes the implant and bone CFUs. There was a significant difference in the total bacterial burden between Group 1 and Group 2.

### Example 2: In Vivo Treatment of Infection with Rabbit PJI Model

This example illustrates the antibacterial activity of an exemplary formulation of WLBU-2 in reducing CFU in vivo on implant bacterial burden and bone bacterial burden with varying concentrations and time of exposure.

TABLE B
Experimental Conditions

| | | Dose | | | | |
|---|---|---|---|---|---|---|
| Group | Test | Volume | Dose | Time of | | Number of |
| No. | material | (mL)/site | (mg/ml) | exposure | | Rabbits |
| 1 | Untreated | 0 | 0 | 0 | | 8 |
| 2 | WLBU-2 | 2 | 1 | 15 | minutes | 8 |
| | (long) | | | | | |
| 3 | WLBU-2 | 2 | 1 | 7.5 | minutes | 7 |
| | (medium) | | | | | |
| 4 | WLBU-2 | 2 | 1 | 5 | minutes | 8 |
| | (short) | | | | | |
| 5 | WLBU-2 | 2 | 0.5 | 15 | minutes | 8 |
| | (long) | | | | | |

A bone tunnel in the tibial canal was created using a drill with a 1.2 mm or 1.6 mm tungsten carbide drill bit. The bone tunnel was then dried and treated to simulate acute human PJI following primary arthroplasty. A Kirschner wire implant was placed in the bone tunnel and the wound closed. Prior to closure of the superficial skin layer, 0.1 mL of 2×10⁶ planktonic bacteria (CFU/rabbit) in saline was injected into the joint space. A closure was performed, and a biofilm was allowed to become established over a period of 2 days.

In Group 1, the joint space was reopened and treated with I&D at 2 days post-infection. In Group 2, the joint space was reopened and treated with I&D and then treated with 2 ml of WLBU-2 at 1.0 mg/ml for 15 minutes. In Group 3, the joint space was reopened and treated with I&D and then treated with 2 ml of WLBU-2 at 1.0 mg/ml for 7.5 minutes. In Group 4, the joint space was reopened and treated with I&D and then treated with 2 ml of WLBU-2 at 1.0 mg/ml for 5 minutes. In Group 5, the joint space was reopened and treated with I&D and then treated with 2 ml of WLBU-2 at 0.5 mg/ml for 15 minutes. The duration of the WLBU-2 treatment was precisely timed by ending the treatment with a PBS flush.

FIG. 4 shows the results of CFU analysis performed on the removed implants. Group 2 had a significantly greater reduction in bacterial burden in comparison to Group 5. A similar reduction in bacterial burden was observed with Group 3.

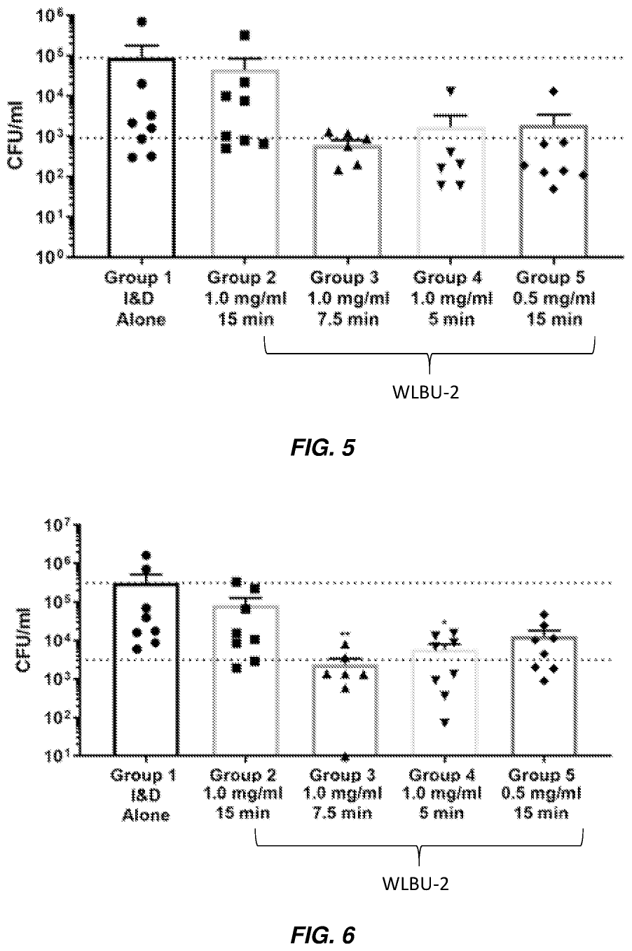

FIG. 5 shows the results of CFU analysis performed with extracted bone samples. There were no significant differences in bacterial burden between treated and untreated samples.

FIG. 6 shows the total CFU which includes the implant and bone CFUs. There were significant differences between Group 1 and Group 3 and between Group 1 and Group 4. Group 3 led to log 2.1 reduction in bacterial burden. Group 4 led to 1.7 log reduction in bacterial burden. There were no significant differences between Group 1 and Group 2 and between Group 1 and Group 5.

### Example 3: Survival Study with Rabbit PJI Model

This example illustrates the antibacterial activity an exemplary formulation of WLBU-2, beta lactam antibiotic, and in combination in reducing CFU in vivo with varying concentrations and time of exposure.

TABLE C
Experimental Conditions

| | | Dose | | | Number |
|---|---|---|---|---|---|
| Group | Test | Volume | Dose | Time of | of |
| No. | material | (mL)/site | (mg/ml) | exposure | Rabbits |
| 1 | Untreated | 0 | 0 | 0 | 7 |
| 2 | Cefazolin | 0 | 10 | BID 5 days | 8 |
| 3 | WLBU-2 | 2 | 1 | 15 minutes | 8 |
| 4* | WLBU-2 + | 2 | 1 + 10 | 15 minutes | 8 |
| | Cefazolin | | mg/kg of | | |
| | | | cefazolin | | |

A bone tunnel in the tibial canal was created using a drill with a 1.2 mm or 1.6 mm tungsten carbide drill bit. The bone tunnel was then dried and treated to simulate acute human PJI following primary arthroplasty. A Kirschner wire implant was placed in the bone tunnel and the wound closed. Prior to closure of the superficial skin layer, 0.1 mL of 2×10⁶ planktonic bacteria (CFU/rabbit) in saline was injected into the joint space. A closure was performed, and a biofilm was allowed to become established over a period of 2 days.

In each group, the joint space was reopened and treated with I&D at 2 days post-infection. Group 1 received neither cefazolin nor WLBU-2. Group 2 received cefazolin but not WLBU-2. Group 3 received WLBU-2 but not the cefazolin. Group 4 received both cefazolin and WLBU-2 treatment. Where applicable, cefazolin was delivered subcutaneously at 10 mg/kg BID for 5 days. Where applicable, WLBU-2 was delivered with 2 ml of WLBU-2 at 1 mg/ml with exposure of 15 minutes. Animals were observed up to 14 days, except for Group 4 which was observed for 28 days. When an animal was sick or needed to be euthanized, the implant and a part of the tibia was collected postmortem and CFU analysis was performed. All surviving animals (except for Group 4) were euthanized on day 14 and the implant and tibia were collected most mortem.

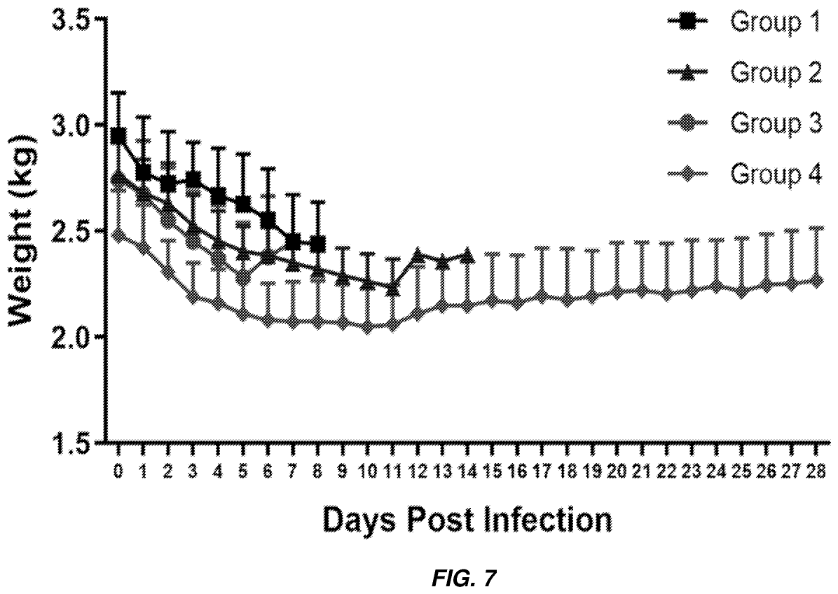

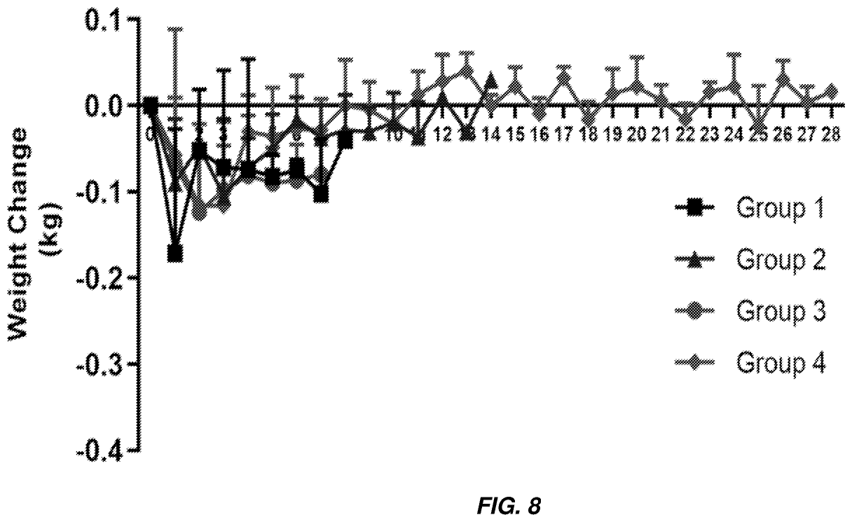

FIG. 7 shows the results of body weight measurements as a function of the number of days post infection. No significance was observed between Group 1 and Group 2, Group 1 and Group 3, and Group 2 and Group 3. Group 4 had the greatest significance in mean body weight in comparison to Groups 1, 2, 3.

FIG. 8 shows body weight change as a function of the number of days post infection. Significant differences were observed between Group 1 and Group 4, and Group 3 and Group 4. There were no significant differences between the other groups. The differences with Group 4 are most likely due to better recovery from infection after treatment.

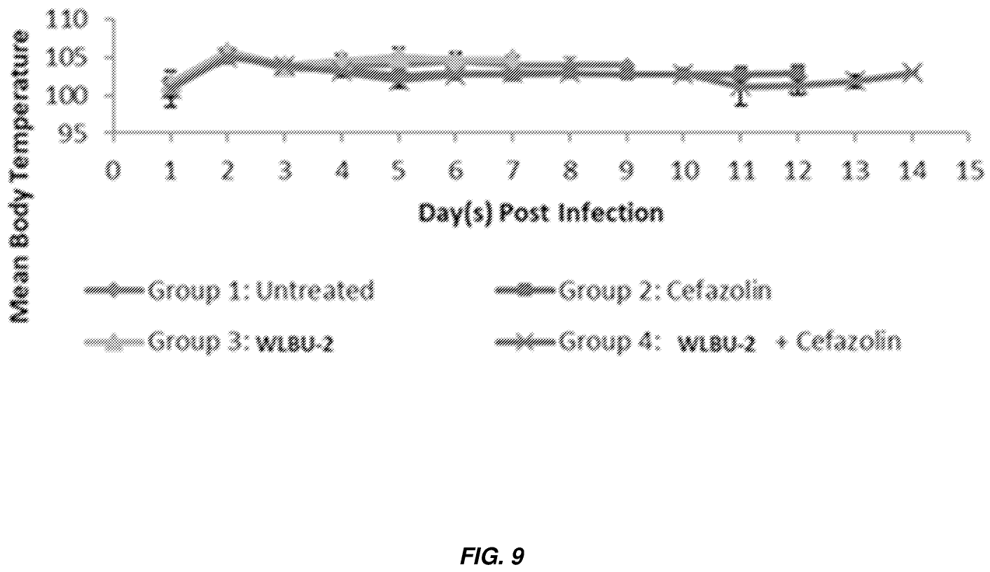

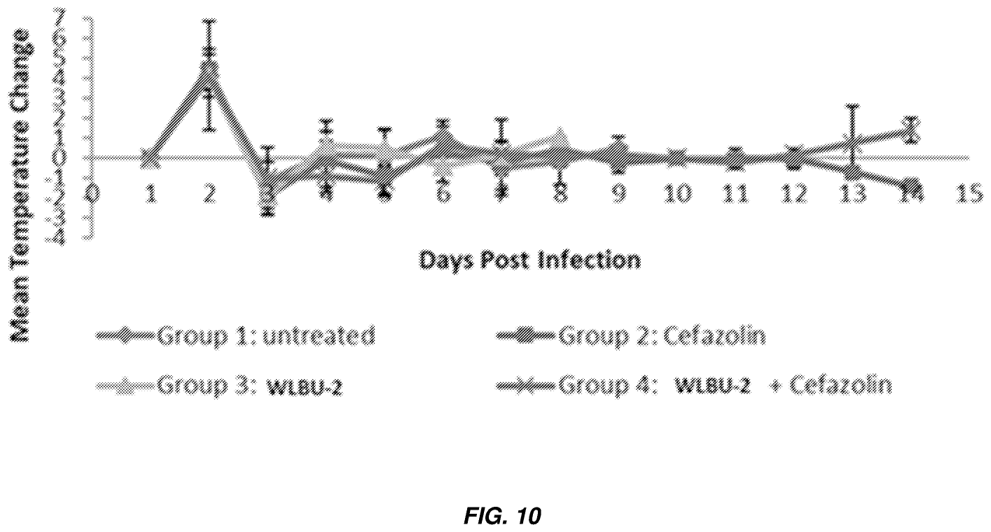

FIG. 9 shows body temperature as a function of the number of days post infection and FIG. 10 shows body temperature change as a function of the number of days post infection. Temperatures were typical for this animal infection model and for the species, strain, sex, and age of animals used on study. If an animal achieved a normal temperature after the 14-day period, temperature was no longer recorded. After infection on Day 0, there is an increase in temperature that is typical for this model.

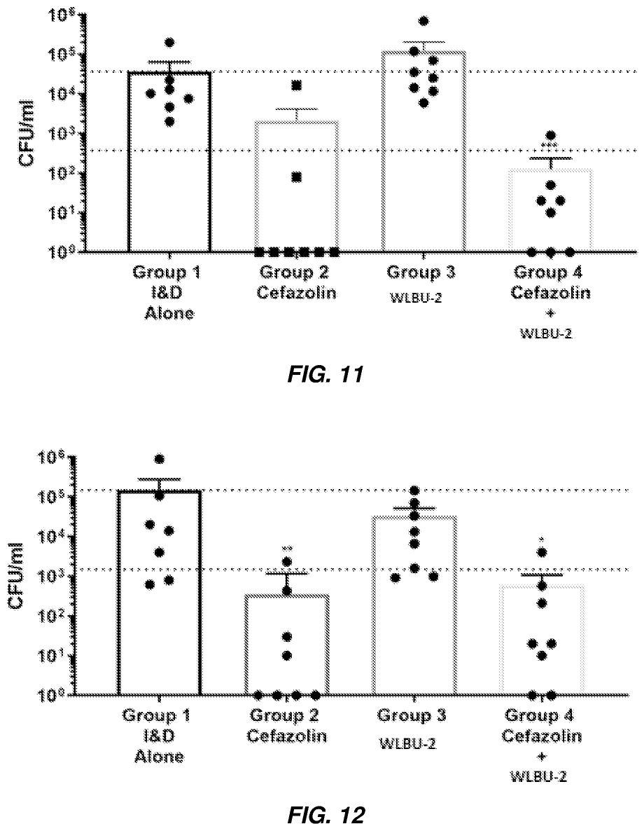

FIG. 11 shows CFU analysis performed with the collected implants. WLBU-2 and cefazolin combination treatment for Group 4 resulted in significant reduction (2.5 log) in bacterial burden.

FIG. 12 shows CFU analysis performed with the collected tibia. There were significant differences between Group 1 and Group 2 and between Group 1 and Group 4. Both Group 2 and Group 4 resulted in 2.6 and 2.4 log reduction in bacterial burden, respectively.

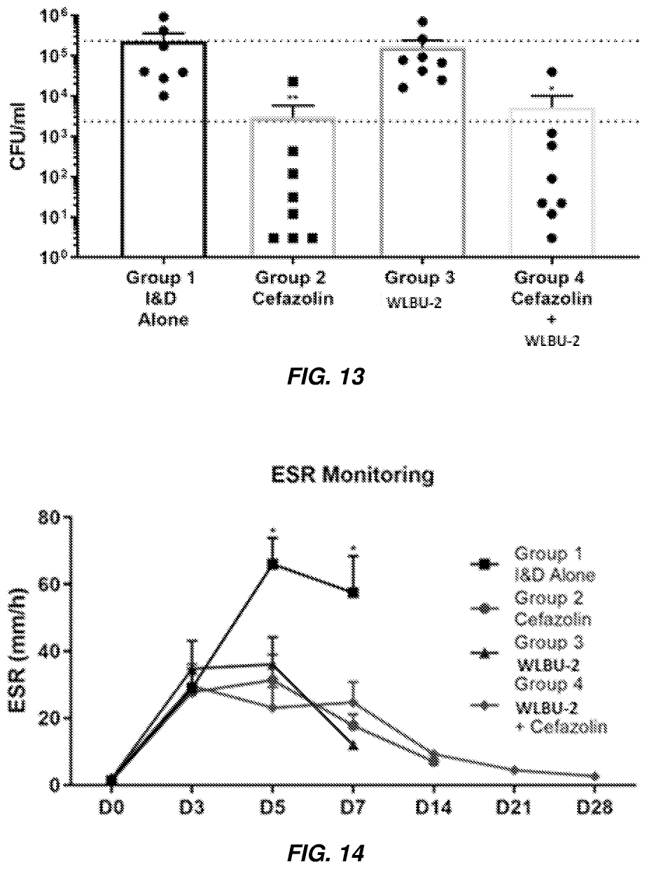

FIG. 13 shows the total CFU which includes the implant and bone CFUs. Significant reductions in total bacterial burden were observed between Group 1 and Group 2 and between Group 1 and Group 4. None of the treatments resulted in a log 2 reduction in total bacteria burden.

FIG. 14 shows the erythrocyte sedimentation rate (ESR) as a function of the number of days post-infection. Group 1 had a significant increase in ESR levels over all other Groups on day 5 and day 7 post infection. ESR levels for Group 4 returned to normal levels (0-20 mm/h) starting on day 14 post infection.

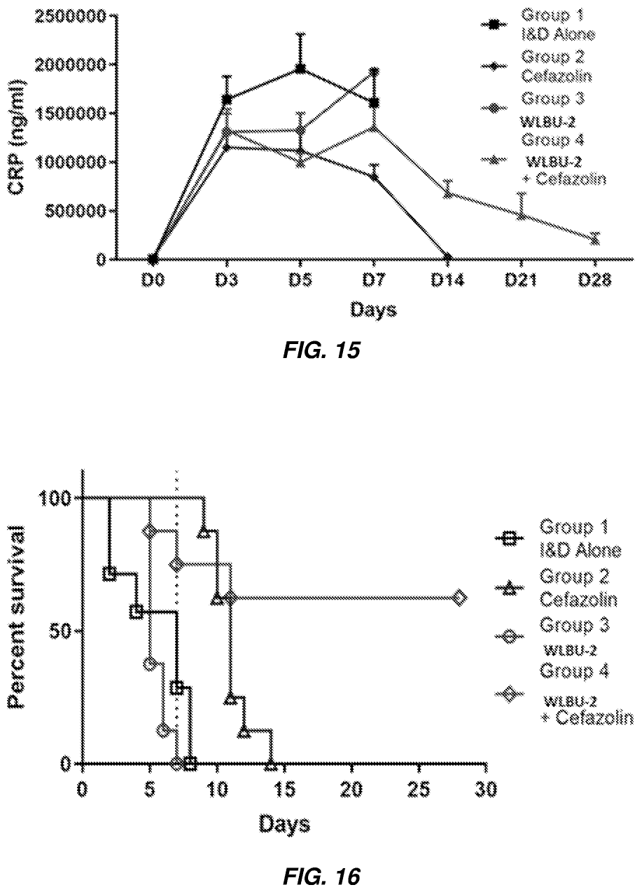

FIG. 15 shows the C-reactive protein (CRP) as a function of the number of days post-infection. CRP levels were similar between treatment groups. There was no statistical significance by Kruskal Wallis tests. Group 4 CRP levels continued to decrease through day 28.

FIG. 16 shows the survival rates of rabbits in each group as a function of the number of days post-infection. Group 4 had a greater overall survival in comparison to Group 1. Group 2 had an improved survival in comparison to Group 1, but the improvement was smaller than the improvement seen with Group 4. Group 3 only had slightly improved survival in comparison to Group 1.

### Example 4: WLBU-2 Biofilm and Planktonic Activity

This example illustrates the antibacterial activity an exemplary formulation of WLBU-2 showing biofilm and planktonic Activity against Multi-Drug Resistant Enterococcus faecium, Staphylococcus aureus, Klebsiella pneumoniae, Acinetobacter baumannii, Pseudomonas aeruginosa, and Enterobacter species (ESKAPE) organisms.

### Methods

Standard Clinical and Laboratory Standards Institute (CLSI) protocol was used to measure the minimum inhibitory concentration (MIC) against each organism. Biofilm model: Implant material was prepared from 0.6 mm diameter stainless steel Kirschner wire and plated at 0.5×106 CFU/ml. Mature biofilm developed over 48 hours, wires were washed, and then treated with 1 mg/mL WLBU-2 (SEQ ID: 10) in PBS (pH 7.4) for 5, 15, and 30 minutes. Quantitative culture (colony forming unit, CFU) analysis was then completed.

### Results

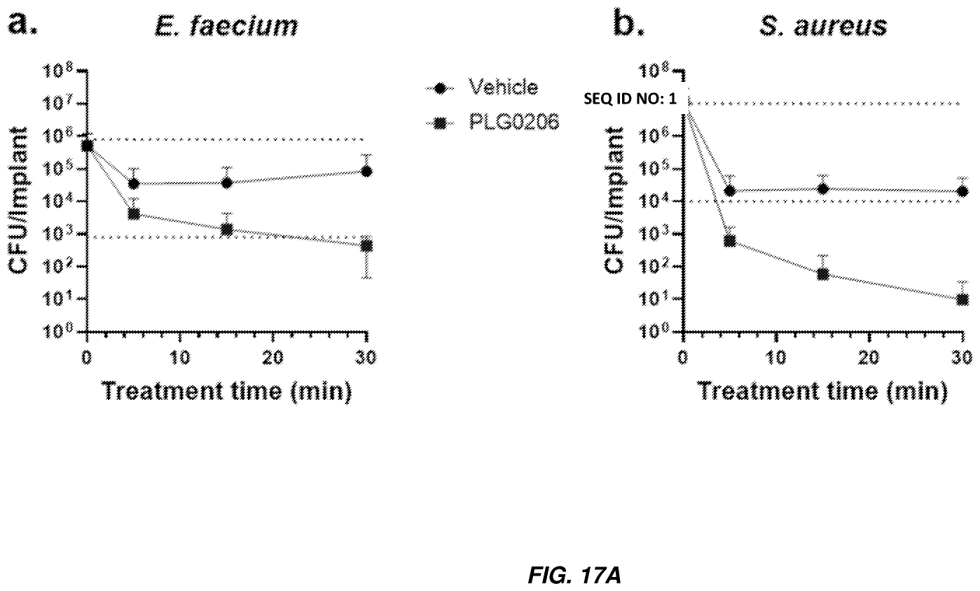

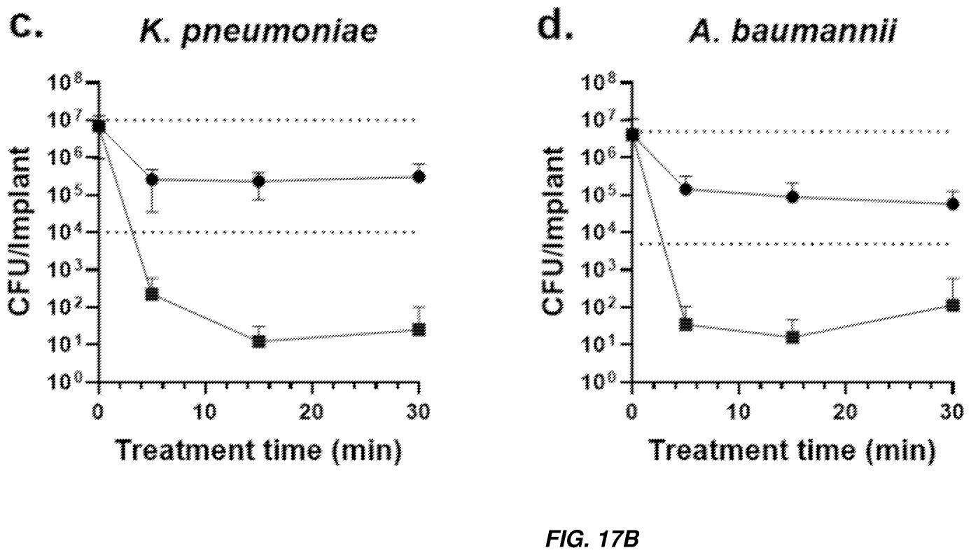

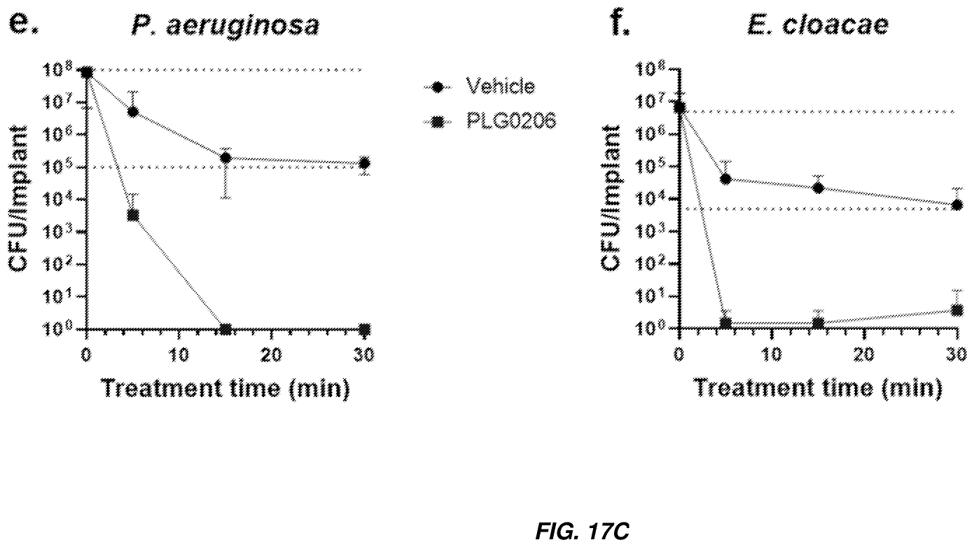

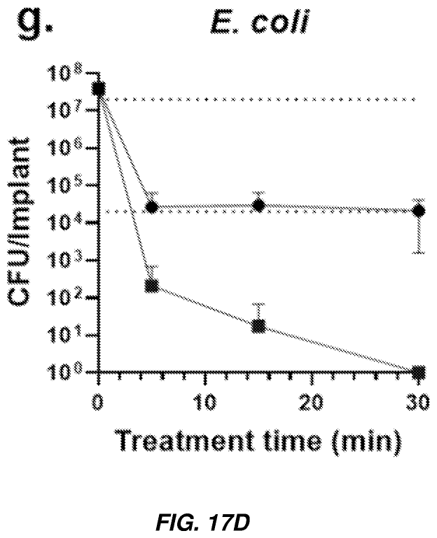

ESKAPE pathogens were selected from a large clinical library of MDR and non-MDR organisms (n>100 organism). WLBU-2 (SEQ ID: 10) displayed similar MIC values to that of clinically used antibiotics levofloxacin (LVX), imipenem (IPM), tigecycline (TIG), linezolid (LZD), vancomycin (VAN), colistin (COL) as shown in Table D. The three most resistant MDR isolates from each organism were then selected for biofilm culture. A similar potent activity was observed with MDR ESKAPE biofilms cultured on implant material (FIG. 17). At 15 minutes, organisms had greater than a 3 log reduction in biofilm mass (E. faecium: 3 log 10; S. aureus: 5 log 10; K. pneumoniae 6 log 10; A. baumannii 5 log 10; P. aeruginosa 8 log 10; E. cloacae 7 log 10; E. coli 5 log 10).

### Discussion

WLBU-2 (SEQ ID: 10) had broad-spectrum planktonic and biofilm activity against ESKAPE pathogens as seen in Table D. WLBU-2 (SEQ ID: 10) had broad spectrum activity against MDR organisms resistant to traditional antibiotics. WLBU-2 (SEQ ID: 10) maintained high activity against MDR ESKAPE biofilms.

TABLE D
WLBU-2 (SEQ ID: 10) broad spectrum activity against planktonic ESKAPE pathogens

| Species | N | WLBU-2 | LVX | IPM | TIG | LZD | VAN | COL |
|---|---|---|---|---|---|---|---|---|
| E. cloacae | 2 | 2-4 | 0.5->4 | 0.25 | 0.5-2 | — | — | .06-.25 |
| S. aureus | 4 | 4->8 | .015-.03 | .015-.03 | 0.25-.05 | 4 | 1 | — |
| K. pneumoniae | 8 | 8->8 | .06->4 | 2->8 | 0.5-2 | — | — | .06-16 |
| A. baumannii | 24 | .25-0.5 | 4->4 | 1->8 | 1-16 | — | — | .12->16 |
| P. aeruginosa | 20 | 8->8 | 0.5->4 | 4->8 | 8->16 | — | — | .12-2 |
| E. coli | 8 | 1-4 | >4 | 4->8 | .25-0.5 | — | — | .06-.25 |

### Example 5: Human Patient Knee Explant Study

This example illustrates the antibacterial activity an exemplary formulation of WLBU-2 in reducing CFU ex vivo.

### Methods

Patients diagnosed with chronic PJI were prospectively enrolled (n=17). All patients met the diagnosis of PJI as defined by the 2018 International Consensus Meeting criteria. The implant was first washed with 50 ml Dulbecco's PBS (dPBS) and submerged ex vivo in an expected clinical exposure of 1 mg/mL WLBU-2 (SEQ ID: 10) in Dulbecco's PBS (dPBS) adjusted to pH 7.4 for 15 minutes. After treatment, the sample was washed with 50 mL of dPBS and sonicated in PBS containing 1% Tween 20 for 5 minutes. After sonication, 100 microliters of the implant sonication fluid was serially diluted, and plated for quantitative culture (CFU analysis). The remaining explanted implant from the same patient served as a control and was processed similarly but without exposure to WLBU-2 (SEQ ID: 10).

### Results

After 15 minutes of treatment with WLBU-2 (SEQ ID: 10), the bacterial burden of both gram-positive and gram-negative bacteria were successfully reduced with more than a 3-log reduction (Table 1). After treatment with WLBU-2 (SEQ ID: 10)., 10 of the 18 infected implants were culture negative. Collectively, infected prosthetics exposed to WLBU-2 (SEQ ID: 10) demonstrated a mean log reduction of 6 (range 2-7.70).

### Discussions

In comparison to untreated samples, 1 mg/ml WLBU-2 (SEQ ID: 10) treatment for 15 minutes successfully reduced the biofilm bacterial burden of both gram-positive and gram-negative bacteria on infected prostheses. The average log reduction was 6, which is enough to be considered bactericidal. These findings support the development of WLBU-2 (SEQ ID: 10) as a local irrigation solution of at least a 1 mg/mL concentration in the wound cavity for 15 minutes for patients undergoing treatment of a PJI occurring after TKA or THA.

TABLE E
Culture and CFU log reduction among bacteria identified from periprosthetic
knee joints exposed and not exposed to WLBU-2 (SEQ ID: 10).

| | | | CFU/mL | CFU/mL | Log |
|---|---|---|---|---|---|
| # | Culture | Multi-drug Resistant (MDR) | untreated | treated | reduction |
| 1 | S. epidermidis | Clindamycin, Erythromycin, Gentamycin, Oxacillin | 5.0 × 10⁷ | 0 | 7.7 |
| 2 | S. epidermidis | Clindamycin, Erythromycin, Gentamycin, Oxacillin | 5.0 × 10⁷ | 0 | 7.7 |
| 3 | S. aureus (MRSA) | Oxacillin, Erythromycin | 5.0 × 10⁷ | 0 | 7.7 |
| 4 | S. hemolyticus | Clindamycin, Gentamicin, Oxacillin, Rifampin, | 7.3 × 10² | 0 | 2.9 |
| | | Sulfa/Trimethoprim | | | |
| 5 | S. aureus (MSSA) | None | 5.0 × 10⁷ | 1.3 × 10² | 3.6 |
| 6 | S. caprae | None | 5.0 × 10⁷ | 0 | 7.7 |
| 7 | E. coli | Ampicillin, Ampicillin/Sulbactam | 5.0 × 10⁷ | 30 | 6.2 |
| 8 | E. coli | Ampicillin, Ampicillin/Sulbactam | 5.0 × 10⁷ | 60 | 5.9 |
| 9 | S. epidermidis | None | 5.0 × 10⁷ | 90 | 5.7 |
| 10 | Haemophilus parain | None | 5.0 × 10⁷ | 0 | 7.7 |
| | fluenzae | | | | |
| 11 | Haemophilus parain | None | 5.0 × 10⁷ | 0 | 7.7 |
| | fluenzae | | | | |
| 12 | E. faecalis | none | 5.0 × 10⁷ | 10 | 6.7 |
| 13 | S. aureus (MRSA) | Oxacillin | 5.0 × 10⁷ | 0 | 7.7 |
| 14 | S. dysgalactiae | none | 4.7 × 10³ | 60 | 4.0 |
| 15 | S. epidermidis | Penicillin | 5.0 × 10⁷ | 0 | 7.7 |
| 16 | S. epidermidis | Oxacillin, Tetracycline, Bactrum sulfa/trimethoprim) | 5.0 × 10⁷ | 0 | 7.7 |
| 17 | S. epidermidis | Oxacillin, Tetracycline, Bactrum (sulfa/trimethoprim) | 5.0 × 10⁷ | 10 | 6.7 |

### Example 6: Human Patient Knee Explant Study

This example illustrates the antibacterial activity an exemplary formulation of WLBU-2 (SEQ ID NO: 10) in reducing colony forming unit (CFU) ex vivo from knee periprosthetics of human patients.

21 adult patients presenting with chronic bacterial periprosthetic joint infection (PJI) of the knee requiring removal of the infected implant components presented to the University of Pittsburgh Medical Center Healthcare System. All patients met the diagnosis of PJI as defined by the 2018 International Consensus Meeting criteria. The infected prosthetics were removed from the 21 patients, despite receiving chronic suppressive oral/intravenous antibiotics, required a 2-stage revision procedure. WLBU-2 was diluted in phosphate buffered solution (PBS) at a concentration of 1 mg/mL and adjusted to pH 7.40. Removed prosthetics were submersed ex vivo with WLBU-2 at an expected clinical concentration of 1 mg/mL for 15 minutes. The WLBU-2 concentration of 1 mg/mL for 15 minutes was chosen based on a series of in vitro time kill studies and murine and rabbit PJI animal models. This dose and duration were non-toxic to local tissue. After the 15-minute exposure to WLBU-2, prosthetics were rinsed with 50 mL of PBS. Then, the explant was placed into PBS+1% Tween 20 and sonicated for 5 minutes. The sonicated solution was then placed for bacterial analysis including culture, antibiotic sensitivity, and CFU enumeration. The remaining explanted implant from the same patient served as a control and was processed similarly but without exposure to WLBU-2.

Table F shows that 17 of 21 (80.9%) patients received prior antibiotics prior to the 2-stage revision procedure. Both Gram-positive and -negative bacteria were identified from removed prosthetics during the 2-stage revision procedure for chronic bacterial PJI. The most common bacteria identified from the prosthesis were S. epidermidis (7/21; 33%), S. aureus (4/21; 19%), and E. coli (3/21; 14%). The majority (12/21; 57%) of the bacteria were resistant to at least one antibiotic. The majority (12/21; 57%) of the chronically infected prosthetics treated ex vivo with (1 mg/mL) WLBU-2 became culture negative. Infected prosthetics exposed to WLBU-2 demonstrated a mean 4 log₁₀ reduction (range 2 to 7) whereas those not exposed to WLBU-2 did not demonstrate any CFU reduction.

The bacteria identified from the prosthetics were consistent with expected strains found in PJI; S. aureus and coagulase-negative staphylococci contribute to between 50 and 60% of PJIs. Coagulase-negative Staphylococcus species, of which S. epidermidis was the most frequently identified members of this group, were ubiquitous members of the human microbiome found on the skin. The relative pathogenicity of these microorganisms is unclear. However, both S aureus and coagulase-negative staphylococci cause PJI primarily through its ability to adhere to prosthetic materials, produce biofilm and production of virulence factors. Another expected bacteria strain commonly found in PJIs, aerobic Gram-negative bacillus is Escherichia coli, was also identified from the prosthetics.

Based on the 80% of the patients receiving prior antibiotics, it may be a factor for most of the bacteria isolated having resistant to at least one antibiotic. Many of the bacteria identified from the prosthetics were susceptible to the antibiotic the patient was taking prior to removal of the prosthetic. Biofilm formation may have protected the bacteria from the antimicrobial and the host immune system, making treatment of infection difficult without a biofilm-directed treatment strategy. These biofilm-directed treatment strategies often lead to surgical intervention, and in many cases including prosthesis removal. The reduced antimicrobial susceptibility of bacteria in biofilms was related to their low growth rate, the presence of resistant bacterial subpopulations, and a microenvironment within the biofilm that impairs antimicrobial activity. Biofilm formation may also explain why some normal floral organisms traditionally considered “harmless” (e.g., coagulase-negative Staphylococci) become pathogens when they are grown in the presence of foreign bodies.

Often, antimicrobials do not have antibiofilm activity. However, WLBU-2 has shown broad-spectrum activity, including activity against multidrug resistant bacteria that cause PJI, potent activity against biofilm, and does not have significant local or systemic toxicity in therapeutic range of dosing in animal models. A mean of 4 log₁₀ reduction in CFU counts was observed after 15 minutes of exposure to WLBU-2 at an expected clinical concentration of 1 mg/mL from prosthetics compared to the prosthetics that were not exposed. These findings support WLBU-2 as a local irrigation solution of at least 1 mg/mL concentration in the wound cavity for 15 minutes in patients undergoing treatment of a P occurring after total knee arthroplasty.

TABLE F
Culture and CFU log reduction among bacteria identified from periprosthetic knee
joints exposed and not exposed to WLBU-2 (SEQ ID NO: 10) for the 21 patients.

| | Preoperative | | | CFU/mL | CFU/mL |
|---|---|---|---|---|---|
| Subject | Antibiotics | Culture | Resistance Pattern | Untreated | Treated |
| 1 | Yes - | S. epidermidis | clindamycin, | 5.00E+07 | 0 |
| | cephalexin | | erythromycin, | | |
| | | | gentamicin, oxacillin | | |
| 2 | Yes - | S. epidermidis | clindamycin, | 5.00E+07 | 0 |
| | cephalexin | | erythromycin, | | |
| | | | gentamicin, oxacillin | | |
| 3 | No | S. aureus | none | N/A | 0 |
| | | (MSSA) | | | |
| 4 | No | S. aureus | oxacillin, | 5.00E+07 | 0 |
| | | (MRSA) | erythromycin | | |
| 5 | Yes | S. hemolyticus | clindamycin, | 7.30E+02 | 0 |
| | | | gentamicin, | | |
| | | | oxacillin, rifampin, | | |
| | | | TMP/SMX | | |
| 6 | Yes - | S. aureus | none | 5.00E+07 | 12,500 |
| | TMP/SMX | (MSSA) | | | |
| 7 | Yes | S. caprae | none | 5.00E+07 | 0 |
| 8 | Yes - | E. coli | ampicillin, | 5.00E+07 | 60 |
| | cefuroxime | | ampicillin/sulbactam | | |
| 9 | Yes - | E. coli | ampicillin, | 5.00E+07 | 30 |
| | cefuroxime | | ampicillin/sulbactam | | |
| 10 | Yes - | E. coli | ampicillin, | 5.00E+07 | 3,510 |
| | cefuroxime | | ampicillin/sulbactam | | |
| 11 | No | S. epidermidis | none | 5.00E+07 | 90 |
| 12 | Yes - | H. parainfluenzae | none | 5.00E+07 | 0 |
| | doxycycline | | | | |
| 13 | Yes - | H. parainfluenzae | none | 5.00E+07 | 0 |
| | doxycycline | | | | |
| 14 | Yes - | E. faecalis | none | 5.00E+07 | 10 |
| | ciprofloxacin | | | | |
| 15 | Yes- | S. aureus | oxacillin, | | |
| | vancomycin | (MRSA) | erythromycin | | |
| 16 | Yes - | S. dysgalactiae | n/a | n/a | 0 |
| | vancomycin | | | | |
| | and cefepime | | | | |
| 17 | Yes - | S. dysgalactiae | n/a | n/a | 60 |
| | vancomycin | | | | |
| | and cefepime | | | | |
| 18 | No | S. epidermidis | penicillin | 5.00E+07 | 0 |
| 19 | Yes - | S. epidermidis | oxacillin, | 5.00E+07 | 0 |
| | cephalexin | | tetracycline, | | |
| | | | TMP/SMX | | |
| 20 | Yes - | S. epidermidis | oxacillin, | 5.00E+07 | 320 |
| | cephalexin | | tetracycline, | | |
| | | | TMP/SMX | | |
| 21 | Yes - | S. epidermidis | oxacillin, | 5.00E+07 | 10 |
| | cephalexin | | tetracycline, | | |
| | | | TMP/SMX | | |

Abbreviations: MRSA, methicillin-resistant S. aureus; MSSA, methicillin-sensitive S. aureus; TMP/SMX, trimethoprim-sulfamethoxazole; CFU, colony forming unit

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby

What is claimed is:

1. A method for treating or preventing periprosthetic joint infection in a patient in need thereof, wherein a prosthetic joint is implanted in said patient; the method comprising administering:
(i) a pharmaceutical composition comprising:
(a) a peptide or pharmaceutically acceptable salt thereof, wherein said peptide has at least 70% sequence identity to a polypeptide sequence of: SA-5 (SEQ ID NO: 1); LSA-5 (SEQ ID NO: 2); WLSA-5 (SEQ ID NO: 3); LBU-1 (SEQ ID NO: 4); LBU-2 (SEQ ID NO: 5); LBU-3 (SEQ ID NO: 6); LBU-3.5 (SEQ ID NO: 7); LBU-4 (SEQ ID NO: 8); WLBU-1 (SEQ ID NO: 9); WLBU-2 (SEQ ID NO: 10); WLBU-3 (SEQ ID NO: 11); WLBU-4 (SEQ ID NO: 12); WR6 (SEQ ID NO: 13); WR12 (SEQ ID NO: 14); WR18 (SEQ ID NO: 15); or WR 24 (SEQ ID NO: 16); and
(b) an aqueous carrier; wherein said pharmaceutical composition is in a form of a liquid, wherein said pharmaceutical composition is locally administered to said prosthetic joint in vivo; and
(ii) an antibiotic or pharmaceutically acceptable salt thereof; and
wherein administration of said pharmaceutical composition and said antibiotic reduces a bacterial burden of said periprosthetic joint infection to a greater extent as compared to administering (i) or (ii) alone.

2. The method of claim 1, wherein said bacterial burden comprises implant bacterial burden, bone bacterial burden, or both.

3. The method of claim 1, wherein said reduction of said bacterial burden is measured as colony-forming unit per milliliter (CFU/mL) by colony-forming unit (CFU) analysis.

4. The method of claim 3, wherein said bacterial burden comprises implant bacterial burden, and wherein said method reduces said CFU/mL by at least 2.5 log.

5. The method of claim 1, wherein said pharmaceutical composition is administered prior to said antibiotic administration.

6. The method of claim 1, wherein said pharmaceutical composition is administered simultaneously with said antibiotic administration.

7. The method of claim 1, wherein said pharmaceutical composition is administered subsequent to said antibiotic administration.

8. The method of claim 1, wherein said periprosthetic joint infection further comprises a biofilm.

9. The method of claim 8, wherein said method reduces said biofilm by at least about 10%, at least about 20%, at least about 30%, or at least about 50%.

10. The method of claim 8, wherein said biofilm is a mature biofilm.

11. The method of claim 8, wherein said method partially disrupts or destroys said biofilm.

12. The method of claim 1, wherein said locally administering comprises irrigating said prosthetic joint with said pharmaceutical composition, wherein said prosthetic joint is exposed.

13. The method of claim 1, wherein said locally administering the pharmaceutical composition to the prosthetic joint in vivo occurs for at least 5 minutes, at least 7.5 minutes, at least 15 minutes, or at least 30 minutes.

14. The method of claim 1, wherein locally administering the pharmaceutical composition to the prosthetic joint in vivo occurs for from about 0.1 minute to about 24 hours or about 0.1 minute to about 60 minutes.

15. The method of claim 1, wherein said antibiotic or pharmaceutically acceptable salt thereof is administered intra-arterially, intravenously, intramuscularly, orally, subcutaneously, rectally, as inhalatory administration, or any combination thereof.

16. The method of claim 15, wherein said antibiotic or pharmaceutically acceptable salt thereof is administered subcutaneously.

17. The method of claim 1, wherein said peptide or pharmaceutically acceptable salt thereof is applied at least about 12 hours prior to said antibiotic or pharmaceutically acceptable salt thereof.

18. The method of claim 1, wherein said peptide or pharmaceutically acceptable salt thereof is applied at most about 30 minutes prior to said antibiotic or pharmaceutically acceptable salt thereof.

19. The method of claim 1, wherein said peptide or pharmaceutically acceptable salt thereof is applied at least about 12 hours subsequent to said antibiotic or pharmaceutically acceptable salt thereof.

20. The method of claim 1, wherein said peptide or pharmaceutically acceptable salt thereof is applied at most about 30 minutes subsequent to said antibiotic or pharmaceutically acceptable salt thereof.

21. The method of claim 1, wherein said peptide or pharmaceutically acceptable salt is WLBU-2 (SEQ ID NO: 10).

22. The method of claim 1, wherein said peptide or pharmaceutically acceptable is WR12 (SEQ ID NO: 14).

23. The method of claim 1, wherein said aqueous carrier comprises phosphate buffered saline (PBS), Dulbecco's PBS, normal saline, water, lactated Ringer's solution, or aqueous sodium bicarbonate.

24. The method of claim 23, wherein said aqueous carrier comprises Dulbecco's PBS, normal saline, water, or aqueous sodium bicarbonate.

25. The method of claim 1, wherein said pharmaceutical composition comprises a pH value of at least about 5 to at least about 10.

26. The method of claim 25, wherein said pharmaceutical composition comprises a pH value of at least about 7.2 to at least about 8.5.

27. The method of claim 1, wherein said peptide or pharmaceutically acceptable salt thereof is present in said pharmaceutical composition at a concentration from at least about 0.01 μg/mL to at least about 100 mg/mL.

28. The method of claim 27, wherein said peptide or pharmaceutically acceptable salt thereof is present in said pharmaceutical composition at a concentration from at least about 1 mg/mL to at least about 10 mg/mL.

29. The method of claim 27, wherein said peptide or pharmaceutically acceptable salt thereof is present in said pharmaceutical composition at a concentration at about 1 mg/mL, about 3 mg/mL, or about 10 mg/mL.

30. The method of claim 1, wherein said antibiotic is a beta-lactam antibiotic.

31. The method of claim 30, wherein said beta-lactam antibiotic is selected from the group consisting of: Amoxillin, Ampicillin, Avibactam, Azlocillin, Aztreonam, Benzathine, Benzylpenicillin, Beta-lactam antibiotic C, Biapenem, Carbenicillin, Cefaclor, Cefadroxil, Cefamandole, Cefapirin, Cefazolin, Cefdinir, Cefditoren, Cefepime, Cefiderocol, Cefixime, Cefoperazone, Cefotetan, Cefotaxime, Cefoxitin, Cefpirome, Cefpodoxime, Cefprozil, Ceftriaxone, Ceftaroline, Ceftazidime, Ceftibuten, Ceftizoxime, Cefuroxime, Cephalexin, Cephalothin, Cephradine, Clavulanic acid, Cloxacillin, Dicloxacillin, Doripenem, Ertapenem, Faropenem, Flucloxacillin, Imipenem, Loracarbef, Mecillinam, Meropenem, Methicillin, Mezlocillin, Nafcillin, Nocardicin A, Oxacillin, Panipenem, Pheneticillin, Phenoxymethylpenicillin, Piperacillin, Procaine penicillin, Razupenem, Sulbactam, Tabtoxinine beta-lactam, Tazobactam, Tebipenem, Temocillin, Ticarillin, Thienamycin, Ticarcillin, Tigermonam, any salts thereof, and any combination thereof.

32. The method of claim 30, wherein said beta-lactam antibiotic is Cefazolin.

33. The method of claim 1, wherein said antibiotic is selected from the group consisting of: Amikacin, Ampicillin, Avibactim, Azithromycin, Aztreonam, Cefepime, Cefpodoxime, Ceftazidime, Ceftriaxone, Ciprofloxacin, Colistin, Daptomycin, Doxycycline, Eravacycline, Gentamicin, Levofloxacin, Linezolid, Meropenem, Penicillin G, Piperacillin, Plazomicin, Sulbactam, Tazobactam, Tetracycline, Tobramycin, Vaborbactam, Vancomycin, any salts thereof, and any combination thereof.

34. The method of claim 1, wherein said antibiotic or pharmaceutically acceptable salt thereof is administered at a concentration of at least about 0.01 μg/mL to at least about 100 mg/mL.

35. The method of claim 34, wherein said antibiotic or pharmaceutically acceptable salt thereof is administered at a concentration of at least about 1 mg/mL to at least about 10 mg/mL.

36. The method of claim 1, wherein said antibiotic or pharmaceutically acceptable salt thereof is administered at a concentration of at least about 0.01 μg/kg to at least about 100 mg/kg.

37. The method of claim 36, wherein said antibiotic or pharmaceutically acceptable salt thereof is administered at a concentration of at least about 1 mg/mL to at least about 10 mg/mL.

38. The method of claim 1, wherein said bacterial burden comprises a bacterial species is selected from the group consisting of Staphylococcus aureus, Staphylococcus epidermidis, Staphylococcus lugdenensis, Staphylococcus haemolyticus, Staphylococcus hominis, Staphylococcus saprophyticus, Staphylococcus simulans, Staphylococcus warnerii, Staphylococcus capitis, Staphylococcus caprae, Staphylococcus pettenkoferi, Streptococcus pyogenes, Streptococcus agalactiae, Streptococcus pneumoniae, Group C streptococci, Streptococcus constellatus, Enterococcus faecalis, Enterococcus faecium, Corynebacterium jeikeium, Lactobacillus acidophilus, Listeria monocytogenes, Escherichia coli, Klebsiella pneumoniae, Klebsiella oxytoca, Acinetobacter baumannii, Acinetobacter nosocomialis, Acinetobacter pittii, Acinetobacter haemolyticus, Acinetobacter radioresistens, Acinetobacter ursingii, Pseudomonas aeruginosa, Enterobacter cloacae, Enterobacter aerogenes, Stenotrophomonas maltophilia, Citrobacter freundii, Citrobacter koseri, Citrobacter sedlakii, Citrobacter braakii, Morganella morganii, Providencia rettgeri, Providencia stuartii, Salmonella typhimurium, Shigella dysenteriae, Moraxella catarrhalis, Neisseria gonorrhoeae, Propionibacterium acnes, Clostridioides difficile, Clostridioides perfringens, Bacteroides fragilis, Prevotella bivia, Eggerthella lenta, Peptostreptococcus anaerobius, Haemophilus parainfluenzae, Staphylococcus haemolyticus, Streptococcus dysgalactiae, and any combination thereof.

39. The method of claim 38, wherein said bacterial species is resistant to at least one antibiotic.

40. The method of claim 39, wherein said at least one antibiotic comprises Ampicillin, Bactrum, Clindamycin, Colistin, Erythromycin, Gentamycin, Imipenem, Levofloxacin, Linezolid, Oxacillin, Rifampin, Sulbactam, Trimethoprim, Tigecycline, Tetracycline, Vancomycin, or a combination thereof.

41. The method of claim 1, wherein said bacterial burden comprises a bacterial selected from the group consisting of Enterococcus species, Staphylococcus aureus, Klebsiella pneumoniae, Acinetobacter species, Pseudomonas species, Enterococcus species, and any combination thereof.

42. The method of claim 1, further comprising debriding said prosthetic joint prior to administration of said pharmaceutical composition.

43. The method of claim 1, wherein said prosthetic joint comprises replacement knee joint, replace hip joint, or replacement shoulder joint.

44. The method of claim 1, further comprising reducing erythrocyte sedimentation rate (ESR) in said subject to a greater extent as compared to administering (i) or (ii) alone.

45. The method of claim 44, wherein said ESR is measured by Westergren method.

46. The method of claim 44, wherein said ESR is measured by Wintrobe method.

47. The method of claim 1, further comprising reducing C-reactive protein expression levels in said subject a greater extent as compared to administering (i) or (ii) alone.

48. The method of claim 1, further comprising increasing a survival rate of said subject.

49. The method of claim 1, wherein said pharmaceutical composition is administered to said subject more than once per day.

50. The method of claim 1, wherein said antibiotic or pharmaceutically acceptable salt thereof is administered to said subject more than once per day.

51. The method of claim 1, wherein said antibiotic or pharmaceutically acceptable salt thereof is administered to said subject more than twice per day.

52. A pharmaceutical composition comprising:
a) a peptide or pharmaceutically acceptable salt thereof, wherein said peptide or pharmaceutically acceptable salt thereof has at least 70% sequence identity to a polypeptide sequence of SA-5 (SEQ ID NO: 1); LSA-5 (SEQ ID NO: 2); WLSA-5 (SEQ ID NO: 3); LBU-1 (SEQ ID NO: 4); LBU-2 (SEQ ID NO: 5); LBU-3 (SEQ ID NO: 6); LBU-3.5 (SEQ ID NO: 7); LBU-4 (SEQ ID NO: 8); WLBU-1 (SEQ ID NO: 9); WLBU-2 (SEQ ID NO: 10); WLBU-3 (SEQ ID NO: 11); WLBU-4 (SEQ ID NO: 12); WR6 (SEQ ID NO: 13); WR12 (SEQ ID NO: 14); WR18 (SEQ ID NO: 15); or WR 24 (SEQ ID NO: 16);
b) an aqueous carrier; and
c) an antibiotic or pharmaceutically acceptable salt thereof;
wherein said pharmaceutical composition is in a form of a liquid.

53. The pharmaceutical composition of claim 52, wherein said peptide or pharmaceutically acceptable salt is WLBU-2 (SEQ ID NO: 10).

54. The pharmaceutical composition of claim 52, wherein said peptide or pharmaceutically acceptable salt is WR12 (SEQ ID NO: 14).

55. The pharmaceutical composition of claim 52, wherein said aqueous carrier comprises phosphate buffered saline (PBS), Dulbecco's PBS, normal saline, water, lactated Ringer's solution, or aqueous sodium bicarbonate.

56. The pharmaceutical composition of claim 55, wherein the aqueous carrier comprises Dulbecco's PBS, normal saline, water, or aqueous sodium bicarbonate.

57. The pharmaceutical composition of claim 52, wherein said pharmaceutical composition comprises a pH value of at least about 5 to at least about 10.

58. The pharmaceutical composition of claim 57, wherein said pharmaceutical composition comprises a pH value of at least about 7.2 to at least about 8.5.

59. The pharmaceutical composition of claim 52, wherein said peptide or pharmaceutically acceptable salt thereof is present in said pharmaceutical composition at a concentration from at least about 0.01 μg/mL to at least about 100 mg/mL.

60. The pharmaceutical composition of claim 59, wherein said peptide or pharmaceutically acceptable salt thereof is present in said pharmaceutical composition at a concentration from at least about 1 mg/mL to at least about 10 mg/mL.

61. The pharmaceutical composition of claim 59, wherein said peptide or pharmaceutically acceptable salt thereof is present in said pharmaceutical composition at a concentration at about 1 mg/mL, about 3 mg/mL, or about 10 mg/mL.

62. The pharmaceutical composition of claim 52, wherein said antibiotic is a beta-lactam antibiotic.

63. The pharmaceutical composition of claim 62, wherein said beta-lactam antibiotic is selected from the group consisting of Amoxillin, Ampicillin, Avibactam, Azlocillin, Aztreonam, Benzathine, Benzylpenicillin, Beta-lactam antibiotic C, Biapenem, Carbenicillin, Cefaclor, Cefadroxil, Cefamandole, Cefapirin, Cefazolin, Cefdinir, Cefditoren, Cefepime, Cefiderocol, Cefixime, Cefoperazone, Cefotetan, Cefotaxime, Cefoxitin, Cefpirome, Cefpodoxime, Cefprozil, Ceftriaxone, Ceftaroline, Ceftazidime, Ceftibuten, Ceftizoxime, Cefuroxime, Cephalexin, Cephalothin, Cephradine, Clavulanic acid, Cloxacillin, Dicloxacillin, Doripenem, Ertapenem, Faropenem, Flucloxacillin, Imipenem, Loracarbef, Mecillinam, Meropenem, Methicillin, Mezlocillin, Nafcillin, Nocardicin A, Oxacillin, Panipenem, Pheneticillin, Phenoxymethylpenicillin, Piperacillin, Procaine penicillin, Razupenem, Sulbactam, Tabtoxinine beta-lactam, Tazobactam, Tebipenem, Temocillin, Ticarillin, Thienamycin, Ticarcillin, Tigermonam, any salts thereof, and any combination thereof.

64. The pharmaceutical composition of claim 63, wherein said beta-lactam antibiotic is Cefazolin.

65. The pharmaceutical composition of claim 52, wherein said antibiotic is selected from the group consisting of: Amikacin, Ampicillin, Avibactim, Azithromycin, Aztreonam, Cefepime, Cefpodoxime, Ceftazidime, Ceftriaxone, Ciprofloxacin, Colistin, Daptomycin, Doxycycline, Eravacycline, Gentamicin, Levofloxacin, Linezolid, Meropenem, Penicillin G, Piperacillin, Plazomicin, Sulbactam, Tazobactam, Tetracycline, Tobramycin, Vaborbactam, Vancomycin, any salts thereof, and any combination thereof.

66. The pharmaceutical composition of claim 52, wherein said antibiotic or pharmaceutically acceptable salt thereof is present in said pharmaceutical composition at a concentration from at least about 0.01 μg/mL to at least about 100 mg/mL.

67. The pharmaceutical composition of claim 66, wherein said antibiotic or pharmaceutically acceptable salt thereof is present in said pharmaceutical composition at a concentration from at least about 1 mg/mL to at least about 10 mg/mL.
